(19) United States
(12) Patent Application Publication
(10) Pub. No.: US 2024/0167001 A1
(43) Pub. Date: May 23, 2024

(54) THREE-DIMENSIONAL TUMOR MODEL OF GLIOBLASTOMA AND BRAIN METASTASIS, METHODS OF MANUFACTURING SAME AND USES THEREOF

(71) Applicant: Ramot at Tel-Aviv University Ltd., Tel-Aviv (IL)

(72) Inventors: Ronit SATCHI-FAINARO, Tel Aviv (IL); Lena NEUFELD, Tel Aviv (IL)

(73) Assignee: Ramot at Tel-Aviv University Ltd., Tel-Aviv (IL)

(21) Appl. No.: 18/424,988

(22) Filed: Jan. 29, 2024

Related U.S. Application Data
(63) Continuation of application No. PCT/IL2022/050824, filed on Jul. 28, 2022.
(60) Provisional application No. 63/226,914, filed on Jul. 29, 2021.

(51) Int. Cl. C12N 5/071

(52) U.S. Cl. CPC C12N 5/0697; C12N 2503/02; C12N 2513/00

(57) ABSTRACT

A three dimensional (3D) model of a glioblastoma tumor made of a synthetic material and a plurality of cell types, including malignant cells and non-malignant cells of the tumor. Methods of forming the 3D tumor model are also provided, as well as systems in which the 3D tumor model can be perfused and fluidly connected to a medium containing immune cells and/or other cells and factors present in the tumor's microenvironment. Methods utilizing the 3D tumor model or the system in, for example, personalized therapy, are also provided.

### RELATED APPLICATIONS

This application is a Continuation of PCT Patent Application No. PCT/IL2022/050824 having International filing date of Jul. 28, 2022, which claims the benefit of priority under 35 USC § 119(e) of U.S. Provisional Patent Application No. 63/226,914 filed on Jul. 29, 2021. The contents of the above applications are all incorporated by reference as if fully set forth herein in their entirety.

The work leading to this invention has received funding from the European Union ERC Programs: 2014-2019 under grant agreement no. 617445; 2019-2024 under grant agreement no. 835227 and 2019-2021 under grant agreement no. 862580.

### FIELD AND BACKGROUND OF THE INVENTION

The present invention, in some embodiments thereof, relates to tumor modeling and, more particularly, but not exclusively, to three-dimensional tumor models featuring structural and functional properties in high match of a respective tumor in a subject, to methods of manufacturing same and to uses thereof in, for example, research, surgery simulation and personalized therapy.

Two-dimensional (2D) cell cultures have a tremendous value in biomedical research in general and in drug screening in particular, however, they do not support tissue-specific and differentiated functions of multiple cell types in disease progression nor do they predict the in-vivo effect of drug activities (1, 2). Moreover, there is an increasing demand to reduce animal testing due to its cost, the long duration required to obtain results, the limitations of in-vivo models in predicting human responses and ethical considerations. In order to overcome the drawbacks of 2D cell culture assays and potentially reduce the need for animal testing, new analytical screening assays in three dimensions (3D) employing human cells are needed (1).

Three-dimensional (3D) cell cultures, mainly made of hydrogels, are composed of either natural extracellular matrix (ECM) molecules, natural polymers or synthetic polymers, which enable cells to polarize and interact with neighboring cells. These 3D culture models are favorable over 2D cell cultures since they represent the tissue better than the 2D monolayer, and thus can be used for studying the molecular basis of tissue function, as well as signaling pathways and drug responsiveness in some disease states. Nonetheless, there are several drawbacks to currently-existing 3D culture models since many of these systems lack multiscale architecture and tissue-tissue interfaces, for example the interface between the vasculature and its surrounding connective tissue and parenchymal cells, which are crucial to the function of nearly all organs (1).

Additive manufacturing (AM) is generally a process in which a three-dimensional (3D) object is manufactured utilizing a computer model of the objects. The basic operation of any AM system consists of slicing a three-dimensional computer model into thin cross sections, translating the result into two-dimensional position data and feeding the data to control equipment which manufacture a three-dimensional structure in a layerwise manner.

Various AM technologies exist, amongst which are stereolithography, digital light processing (DLP), and three-dimensional (3D) printing such as 3D inkjet printing, extrusion printing, electrospinning, etc. Such techniques are generally performed by layer by layer deposition and solidification of one or more building materials, typically photopolymerizable (photocurable) materials.

Stereolithography, for example, is an additive manufacturing process which employs a liquid ultraviolet (UV)-curable building material and a UV laser. In such a process, for each dispensed layer of the building material, the laser beam traces a cross-section of the part pattern on the surface of the dispensed liquid building material. Exposure to the UV laser light cures and solidifies the pattern traced on the building material and joins it to the layer below. After being built, the formed parts are immersed in a chemical bath in order to be cleaned of excess building material and are subsequently cured in an UV oven.

In three-dimensional printing processes, for example, a building material is dispensed from a dispensing head having a set of nozzles to deposit layers on a supporting structure. Depending on the building material, the layers may then be cured or solidified using a suitable device.

The building materials may include modeling materials and support materials, which form the object and the temporary support constructions supporting the object as it is being built, respectively.

The modeling material (which may include one or more materials) is deposited to produce the desired object/s and the support material (which may include one or more materials) is used, with or without modeling material elements, to provide support structures for specific areas of the object during building and assure adequate vertical placement of subsequent object layers, e.g., in cases where objects include overhanging features or shapes such as curved geometries, negative angles, voids, and so on.

Both the modeling and support materials are preferably liquid at the working temperature at which they are dispensed, and subsequently hardened, typically upon exposure to curing energy (e.g., UV curing), to form the required layer shape. After printing completion, support structures are removed to reveal the final shape of the fabricated 3D object.

Additive manufacturing has been first used in biological applications for forming three-dimensional sacrificial resin molds in which 3D scaffolds from biological materials were created (1).

3D bioprinting is an additive manufacturing methodology which uses biological materials, chemicals and cells that are printed layer-by-layer with a precise positioning and a tight control of functional components placement to create a 3D structure (3). 3D bioprinting technology is favorable over currently-existing 3D culture models since it creates objects that sense and respond to their environment.

Organ printing, a novel approach in tissue engineering, applies layered computer-driven deposition of cells and gels to create complex 3D cell-laden structures. It shows great promise in regenerative medicine, because it may help to solve the problem of limited donor grafts for tissue and organ repair.

3D bioprinting goal is to create tissues that mimic their natural structure, and are composed of multiple cell types with different extracellular matrices and functional microvasculature. This goal can be approached by two ways (4):
1. Using a scaffold (synthetic or natural) or a decellularized organ which is seeded with cells and then matured in a bioreactor. This approach is useful mainly for generation of avascular tissues.
2. Assembling “building blocks” that mimic the native tissue functional units into larger tissue constructs. This approach allows the inclusion of microvasculature and direct fabrication of a functional tissue-architecture.

A 3D organ model has already been successfully used to form bones as well as cartilaginous structures, such as ears and tracheas (5).

Additional related art includes U.S. Patent Application Publication Nos. 20130190210, 20150282885 and 20150246072; Hinton et al. 2015 Sci. Adv. I:e1500758; Homan et al. 2016 6:34845 | DOI: 10.1038/srep34845; Miller et al., Nat. Let. 2012 DOI: 10.1038/NMAT3357; Wu et al. Adv. Mat. 2011:23:H178-H183; Ozbolat et al. 3D BioprintingL Fundamentals, Principles and Applications. Academic Press, 2016; and WO2018/127850.

### SUMMARY OF THE INVENTION

According to an aspect of the invention there is provided a three dimensional (3D) model of a glioblastoma (GB) tumor or a brain metastasis comprising a hardened first synthetic material and a plurality of cell types of the tumor or the brain metastasis and microenvironment thereof, the plurality of cell types comprising endothelial cells, pericytes, astrocytes, microglia cells and GB tumor cells or metastatic cells.

According to some embodiments, at least a portion of the model is bioprinted.

According to some embodiments, a first mixture of the plurality of cell types form the tumor and a microenvironment thereof, and a second mixture of the plurality of cell types form a vasculature which allows perfusion, the first and second mixtures being different from one another.

According to some embodiments, the plurality of cell types are of a human and/or mouse origin.

According to some embodiments, the endothelial cells and pericytes form a vasculature which allows perfusion.

According to some embodiments, a ratio of the endothelial cells and the pericytes in the vasculature ranges from 2:1 to 10:1.

According to some embodiments, the 3D model is characterized by at least one of:
(i) porosity of 1-40 μm;
(ii) cell viability for at least 4 weeks under physiological conditions;
(iii) stiffness of 5-25 kPa Young's Modulus;
(iv) swelling equilibrium of 5-25%;
(v) perfusability for at least 1 day; and/or
(vi) activation of the microglia and/or astrocytes.

According to some embodiments, the vasculature is configured to provide perfusion at a range of shear stress of 0.01-100 dyn/cm2 or 5-1000 μl/min, for a vessel of 1 mm in diameter of the vasculature.

According to some embodiments, the vasculature is configured to provide perfusion at shear stress of 25 μl/min for a vessel 1 mm in diameter of the vasculature.

According to some embodiments, the perfusion is performed in a growth medium. According to some embodiments, the tumor cells are non-immortalized patient derived. According to some embodiments, the tumor cells are of a GB cell line.

According to some embodiments, the vasculature is for perfusing peripheral blood cells (PBMCs) and/or a drug.

According to some embodiments, a density of the tumor cells in the model is between is between 0.1×10⁵-1×10⁸ cells/ml

According to some embodiments, a ratio of the tumor cells to the astrocytes and microglia cells is in the range of 20:1 to 1:10.

According to some embodiments, a ratio of the tumor cells to the astrocytes and microglia cells is in the range of 1:10 to 10:1.

According to some embodiments, a ratio of the tumor cells to the astrocytes and microglia cells is in the range of 1:1 to 5:1.

According to some embodiments, the 3D model being embedded in an extracellular matrix.

According to some embodiments, the extracellular matrix comprises a synthetic material.

According to some embodiments, the extracellular matrix comprises Matrigel™.

According to some embodiments, the extracellular matrix is naturally occurring.

According to some embodiments, the plurality of cell types exhibit a gene expression pattern which is more similar to that of the tumor in-vivo as compared to that of a 2D culture.

According to some embodiments, the first synthetic material comprises a hardened form of a curable material.

According to some embodiments, the first hardened synthetic material comprises a polymeric material.

According to some embodiments, the first synthetic material comprises a synthetic polymer.

According to some embodiments, the first synthetic material comprises fibrin and an anionic polymer cross-linked to one another.

According to some embodiments, the fibrin is formed upon enzymatically-catalyzed polymerization of fibrinogen.

According to some embodiments, the fibrin and the anionic polymer are cross-linked to one another upon an enzymatic reaction.

According to some embodiments, the anionic polymer comprises gelatin.

According to some embodiments, an amount of the anionic polymer, an amount of the fibrin and a degree of the cross-linking are selected so as to provide a pre-determined mechanical and/or physical property of the GB tumor model (the property being selected, for example, in accordance with a respective property of a patient's derived glioblastoma tumor).

According to some embodiments, the first synthetic material is formed from a curable formulation that comprises the anionic polymer and fibrinogen.

According to some embodiments, the curable formulation further comprises at least one enzyme for promoting the cross-linking and/or for generating the fibrin.

According to some embodiments, a concentration of the anionic polymer (e.g., gelatin) in the curable formulation ranges from 1 to 20, or from 3 to 18, or from 3 to 10, or from 1 to 10, or from 4 to 10, or is 6, % by weight of the total weight of the curable formulation.

According to some embodiments, a concentration of the fibrinogen in the curable formulation ranges from 0.1 to 10, or from 0.1 to 5, or from 0.5 to 5, or from 1 to 5, or from 0.1 to 3, or from 0.5 to 2, or from 0.5 to 1.5, or is 1, % by weight of the total weight of the curable formulation.

According to some embodiments, the enzyme is selected from thrombin, transglutaminase and a mixture thereof.

According to some embodiments, a concentration of the thrombin, if present, in the curable formulation, ranges from 0.1 to 5, or from 0.1 to 4, or from 0.1 to 3, or from 0.1 to 2, or from 0.1 to 1, or is 0.5, U/ml.

According to some embodiments, a concentration of the transglutaminase, if present, in the curable formulation, ranges from 0.1 to 50, 0.1 to 10, or from 1 to 10, or from 1 to 5, or is 3, % by volume of the total volume of the formulation.

According to some embodiments, the first synthetic material comprises fibrin, gelatin, thrombin and transglutaminase (TG).

According to some embodiments, the first synthetic material comprises 1% fibrin, 6% gelatin, 0.5 U/ml thrombin, 3% TG, 2.5 mM CaCl₂.

According to an aspect of the invention there is provided a curable formulation, comprising fibrinogen, gelatin, thrombin and transglutaminase.

According to an aspect of the invention there is provided a method of producing the 3D model as described herein comprising:
(a) dispensing at least one layer of astrocytes, microglia cells and GB tumor cells or metastatic cells comprised in a first synthetic material;
(b) forming a vascularization pattern on the at least one layer using a second synthetic material which is liquefiable to allow formation of perfusable tubular vessels comprising lumen; wherein step (a) is performed prior to and following step (b);
(c) dispensing the second synthetic material to form the lumen;
(d) applying endothelial cells and pericytes into the lumen to form blood vessels.

According to some embodiments, the dispensing is by printing or casting.

According to some embodiments, the forming is by printing.

According to an aspect of the invention there is provided a system comprising the perfused 3D model of any one of claims and a container in fluid communication with the 3D model as described herein.

According to some embodiments, the method further comprises a peristaltic pump for effecting the fluid communication.

According to some embodiments, the fluid communication is at a shear rate of 0.01-100 dyn/cm2 or 5-1000 μl/min for a printed vessel of 1 mm in diameter, preferably 25 μl/min.

According to an aspect of the invention there is provided a method of screening for an anti-cancer treatment regimen suitable for a patient suffering from glioblastoma, the method comprising:
subjecting a 3D model of a tumor as described herein to the anti-cancer treatment regimen; and
determining a presence of an anti-cancer effect of the anti-cancer treatment regimen at a personalized manner.

According to some embodiments, the anti-cancer treatment regimen is selected from the group consisting of a chemotherapy, a radiotherapy and a hormonal therapy.

According to some embodiments, the anti-cancer treatment regimen comprises a combination therapy.

According to an aspect of the invention there is provided a method of screening for an anti-cancer treatment regimen suitable for a patient suffering from glioblastoma, the method comprising:
subjecting a system as described herein to the anti-cancer treatment regimen; and
determining a presence of an anti-cancer effect of the anti-cancer treatment regimen at a personalized manner.

According to some embodiments, the anti-cancer treatment regimen is selected from the group consisting of a chemotherapy, a radiotherapy and a hormonal therapy.

According to some embodiments, the anti-cancer regimen comprises an immune check point modulator.

According to some embodiments, the anti-cancer regimen comprises a TSP-1 inhibitor.

According to an aspect of the invention there is provided a method of characterizing a tumor, the method comprising:
providing the 3D model of the tumor as described herein;
isolating cells of the model;
in vitro or in vivo culturing the cells.

According to some embodiments the method further comprises subjecting the cells to an anti-cancer treatment during the culturing.

Unless otherwise defined, all technical and/or scientific terms used herein have the same meaning as commonly understood by one of ordinary skill in the art to which the invention pertains.

Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E: Bio-mechanical characterization determined the bio-ink composition which best mimics the elasticity and composition of the brain tissue. A. Oscillation time evaluation of fibrin 3D-bio-ink formation at different Th concentrations with 3% w/v TG at 37° C. (average shown of n=3 per group). B. Young's modulus of fibrin 3D-bio-ink at different concentrations of gelatin [3%, 6% and 12% w/v] with 3% w/v TG and 0.5 U/ml Th as a clear bio-ink and as a cell-laden bio-ink composed of PD-GB4 or GL261 1×10⁶ cells/ml. No significant differences were measured at the same gelatin concentration, however significant differences were evaluated at different gelatin concentration (3% w/v versus 6% w/v p=0.0037, 6% w/v versus 12% w/v p=0.0003, 3% w/v versus 12% w/v p<0.0001 by two-way ANOVA Tukey's multiple comparisons test) (n=8-13 per group). C. Swelling at equilibrium of fibrin 3D-bio-ink at different concentrations of gelatin [3%, 6% and 12% w/v] with 3% w/v TG and 0.5 U/ml Th. No significant differences were measured between 3% w/v and 6% w/v gelatin concentration; a significant difference was evaluated between 3% w/v and 6% w/v gelatin concentration to 12% w/v (p=0.0014 and p=0.0011, respectively, by one-way ANOVA Tukey's multiple comparisons test) (n=8 per group). D. Growth curves at different concentrations of gelatin [3%, 6% and 12% w/v] with 3% w/v TG and 0. 5 U/ml Th of murine GL261 (upper panel, 3% w/v versus 6% w/v p<0.00001, 6% w/v versus 12% w/v p<0.00001, 3% w/v versus 12% w/v p=0.002 by t-test) and GB patient-derived (PD-GB4) (lower panel, 3% w/v versus 6% w/v p=0.0004, 6% w/v versus 12% w/v p=0.0004, by t-test) in fibrin 3D-bio-ink (1×10⁶ cells/ml) (n=4 per group). E. Representative images demonstrating the morphology of mCherry-labeled GL261 (upper panel) and iRFP-labeled PD-GB4 cells (lower panel) following 14 days in fibrin 3D-bio-ink (6% w/v gelatin, 3% w/v TG and 0.5 U/ml Th). Cells were analyzed by live confocal Z-stack imaging of the whole bio-ink, and by fluorescence imaging and H&E staining of bio-ink sections (n=3-4 per group). Scale bars: 100 μm.

FIGS. 2A-F show that a 3D brain mimicking bio-ink is biocompatible and promotes long-term cell viability of GB cells and brain stromal cells. A. Representative immunostaining images of GFAP (upper panel, in green) or IBA1 (lower panel, in green) in fibrin 3D-bio-ink seeded with GB and stromal cells co-cultured for 7 days. Images depict Hoechst-stained nucleus (in blue), iRFP-labeled patient-derived GB cells (PD-GB4; in cyan). Scale bar: 100 μm. B. Growth curves of patient-derived GB cells (PD-GB4), alone or co-cultured with hAstro (1:1 ratio) in fibrin 3D-bio-ink (p=0.004, t-test; n=4 per group). C. The invasion of iRFP-labeled PD-GB4 cells from the inner core to the surrounding area in the absence or presence of hAstro (1×10⁶ cells/ml) was evaluated by fluorescent microscopy imaging. The Invasion was calculated as the total area density in outer bio-ink and quantified using ImageJ by RLU (p=0.004, t-test; n=12 per group). Representative images of cell invasion are shown; dashed lines delineate the edge between the core and the surrounding tissue according to the images on day 1. Scale bar: 100 μm. D. SEM images of acellular fibrin 3D-bio-ink (upper panel-left), cell-laden fibrin 3D-bio-ink composed of patient-derived PD-GB4 cells, hAstro and hMG cells (lower panel-left), healthy hemisphere of a

C57BL/6 mouse (upper panel-right) and GL261 tumor containing hemisphere of the same mouse (lower panel-right). Scale bar: 10 μm. The pore size diameter of each group was quantified using ImageJ (n=2-20 photos per group, n=13-170 measurements in each photo) and showed that enlarged and diverse pores sizes characterized the cell-laden bio-ink and brain tissue bearing GB tumor. E. Growth curves of human primary astrocytes and human primary microglia in 3D fibrin bio-ink. F. Growth curves of GL261, U373, PD-GB1 and U-87MG GB cell types co-cultured with murine or human astrocytes (according to the cell origin) in fibrin 3D-bio-ink (n=4 per group).

FIGS. 3A-G show a fibrin brain mimicking 3D-bio-ink integrated with 3D engineered printed perfusable vascular network. A. Schematic illustration of the 3D-bioprinting model multi-stage process. B. 3D-printed Pluronic-based vascular bio-ink (in cyan) on top of 3D-printed layers of fibrin 3D GB-stroma bio-ink (in white). C. 3D-bioprinted vascularized GB model scaled in a metal frame showing the complete perfusion chip. D. The vascularized 3D-bioprinted GB model is connected to a peristaltic pump through a tubing system, placed in a designated incubator. E. Tiled Z-stack confocal microscopy images of the 3D-printed penta-culture vascularized GB model. Blood vessels are lined with iRFP-labeled hPericytes (in cyan) together with mCherry-labeled

HUVEC (in red) (107 cells/ml; 4:1 ratio) and surrounded by azurite-labeled PD-GB4 (in blue), GFP-labeled hAstro (in green) and non-labeled hMG (2.1×10⁶ cells/ml; 1:1:0.1 ratio). The dashed box represents coronal cross section plane of the vessel. F. Fluorescence microscopy images of the 3D-bioprinted vascularized GB model before (upper panel) and after (lower panel) perfusion of 70 kDa Dextran-FITC. The 3D-bioprinted model is composed of fluorescently labeled vascular network (mCherry-labeled HUVEC, iRFP-labeled hPericytes) surrounded by non-labeled GB-bio-ink (hAstro, PD-GB4, hMG). G. Schematic illustration of the fluid flow inside the 3D printed model via the peristatic pumps, media reservoir, waste reservoir and filters (0.22 μm).

FIGS. 4A-F show that fibrin 3D-bio-ink reproduced the dormancy phenomenon of two GB human cell types, which thus far could only be observed in SCID mice and not in 2D culture. A. Schematic illustration of T98-G and U-87MG human dormancy models. B. In-vivo growth kinetics of dormant (T98G-D and U-87MG-D) and fast-growing (T98G-F and U-87MG-F) cell types. n=4 in the T98G-F group and n=3 in the T98G-D group. * Values for U-87MG growth in mice were averaged from data previously presented (29). C. Cell growth evaluation of both GB pairs in 2D culture. n=3 per group. D. Cell invasion evaluation of both GB pairs in 2D culture. n=3 per group. E. Growth kinetics of both dormant and fast-growing cell types were evaluated in fibrin 3D-bio-ink. n=4 per group. F. Cell invasion ability in fibrin 3D-bio-ink was quantified using ImageJ. n=12 per group. Representative fluorescent images of the invasion from the core tumor model to the surrounding area are presented. Scale bar: 100 μm. Dashed lines delineate the edge between the core and the surrounding tissue according to the images on day 1.

FIGS. 5A-C show that treatment with SELPi resulted in a significant reduction in GB cell proliferation in fibrin 3D-bio-ink compared to 2D culture. A-C. Response of PD-GB4 (A), T98G-F (B) and U-87MG-F (C) to treatment with SELPi in 2D culture (left panel, n=3 per group) and in 3D-bio-ink (centre, n=8/12 per group), representative images of labeled cells at the end of evaluation, scale bar: 100 μm. Flow cytometry analysis of P-selectin expression of cells grown in 2D culture and in fibrin 3D-bio-ink (right panel, n=3 per group).

FIGS. 6A-D show RNA-seq analysis demonstrating higher similarities between the gene expression of GL261 grown in fibrin 3D-bio-ink and GL261 cells grown in mice compared to those grown in 2D culture. A. PCA analysis showing gene expression profile derived from 2D culture, 3D-bio-ink and GB tumors in mice in-vivo (n=3 per group). B. Euclidian distance matrix between samples showing a closer distance between the iv-vivo samples and the fibrin 3D-bio-ink compared to increased distance between the in-vivo samples and the 2D culture. C. Summary comparison between the Euclidian distance of the 3D-bio-ink and the 2D culture to the in-vivo samples (p=2.8×10^{7,} t-test). D. A comparison of gene expression levels of enriched pathways, displaying similarly high levels of expression both in fibrin 3D-bio-ink an in-vivo.

FIGS. 7A-C show that different IC₅₀ values were observed in fibrin 3D bio-ink using different patient-derived GB cells as opposed to similar values observed in 2D cultures. A. Proliferation curves of 3 patient-derived cells in 2D culture in the presence of TMZ. B. Proliferation curves of 3 patient-derived cells in fibrin 3D-bio-ink samples in the presence of TMZ. C. Summarized values of IC₅₀ values.

FIGS. 8A-B show a breast cancer metastatic model in the brain microenvironment. A. As a preliminary brain metastasis model we have created a simplified vascular structure using a needle inside our perfusion chip. B. The outer fibrin 3D-bio-ink contained the human metastatic breast cancer cells, MDA-MB-231, labeled with GFP (1×10⁶ cells/ml) mixed with unlabeled hAstro (1×10⁶ cells/ml) and hMG (1×10⁵ cells/ml). Next, a mixture of mCherry-labeled HUVEC (8×10⁶ cells/ml) and iRFP-labeled human microvascular brain pericytes (2×10⁶ cells/ml) at 4:1 ratio was injected into the vessel and incubated for 3 days in rotation.

FIG. 9 describes the bridging the translational gap from bedside to bench and back. Schematic illustration of the methodological approach using a perfusable micro-engineered vascular 3D-bioprinted tumor model for drug screening and target discovery.

### DESCRIPTION OF SPECIFIC EMBODIMENTS OF THE INVENTION

The present invention, in some embodiments thereof, relates to tumor modeling and, more particularly, but not exclusively, to three-dimensional tumor models featuring structural and functional properties in high match of a respective tumor in a subject, to methods of manufacturing same and to uses thereof in, for example, research and personalized therapy.

Embodiments of the invention relate to a method of additive manufacturing of a 3D tumor model that senses and responds to its microenvironment. This tumor model can be composed of multiple cell types with different extracellular matrices; taking into consideration the surrounding microenvironment, including, for example, blood vessels and stroma consisting of fibroblasts and immune cells. It is patterned based on a 3D intravital imaging data (e.g. MRI or a CT scan), such that it can closely mimic the actual natural anatomical structure, environment and conditions under which a malignant tumor grows. Such a 3D model of the tumor can be used in various research and clinical applications including gaining in depth insight into tumor development, drug development and personalized therapy.

To date, discovery, development and evaluation of new therapies is performed using human cancer cells grown in 2D culture dish, which are frequently subjected to mechanical stress and bounded to the rigid plastic, followed by in-vivo testing in immunodeficient mice. These experimental settings constitute a fundamental hurdle in translation of preclinical discoveries into clinical practice, with only one out of 10,000 new potential drugs successfully reaching the market. Therefore, there is an unmet need for alternative drug discovery and screening platforms that can accurately predict clinical response to therapies.

Whilst conceiving embodiments of the invention and reducing them to practice, the present inventors devised a perfusable 3D-bioprinted brain tumor model based on biocompatible polymers containing tumor cell, stromal cells and vascularization forming cells. This model recapitulates the tumor, vascularation and tumor microenvironment (TME).

As is described hereinbelow and in the Examples section which follows, the present inventors formulated and characterized a brain mimicking fibrin 3D-bio-ink. The fibrin 3D-bio-ink's gelation time was optimized as a function of the concentrations and ratios of the gelatin and the crosslinking enzymes, thrombin (Th) and Transglutaminase (TG). Rheological and mechanical characterization confirmed that the resultant fibrin 3D-bio-ink shares similar mechanical properties of GB brain tissue (33). Furthermore, the fibrin 3D-bio-ink exhibited moderate swelling degree of approximately 5% to 20%, a desirable aspect of the bio-ink characteristics, since low swelling degree (less than 40%) is important to accurately maintain the printed model dimensions and the desired optimal mechanical properties (53). The swelling at equilibrium of the fibrin 3D-bio-ink suggests that it has the potential for high water content, high porosity and efficient nutrients diffusion, same as other hydrogels with similar mechanical properties. Moreover, the fibrin 3D-bio-ink demonstrated a long-term culture capability, as it supported GB cell viability for up to 5 weeks. Similar cell morphology was exhibited by live confocal Z-stack images and in histological H&E staining of GB slides from mice (55). As the brain TME plays a vital role in tumor progression (24), both astrocytes and microglia were incorporated into the ink, which both have been shown to have a role in GB progression and constitute the majority of glial cells in the brain. It was found that fibrin 3D-bio-ink supports the viability of GB TME cells for up to 8 weeks post seeding. Remarkably, patient-derived GB cells proliferated and invaded more rapidly when co-cultured with hAstro in fibrin 3D-bio-ink. Furthermore, immunostaining showed that hAstro and hMG in the 3D-GB-bio-ink were in their activated state, expressing GFAP and IBA1, respectively.

To further recapitulate tumor architecture and cellular heterogencity, the present inventors 3D-bioprinted GB-stroma bio-ink composed of GB cells, astrocytes and microglia, together with a 3D-printing vascular bio-ink, composed of the thermo-reversible Pluronic F127, to generate a vascular lumen coated with endothelial cells and pericytes, generating a 3D-bioprinted penta-culture. This 3D-bioprinted tumor model, was perfused through its hollow channels by a peristaltic pump through a tubing system over 5 days. Metabolized cell media was pumped through the outlet vessel. This 3D perfusion chip allows long-term flow, imaging and drug response assessment. This is the first report of a perfusable 3D-bioprinted engineered GB penta-culture.

The present system allows incorporation of peripheral blood mononuclear cells (PBMC), circulating tumor cells, and/or a variety of anti-cancer drugs which can be redirected either into a waste container after they perfuse through the 3D-bioprinted model, collected for further analysis or returned back to the inlet vessel, generating a closed circulatory system. Hence, perfusable 3D-printed models of some embodiments of the invention can be manufactured on demand to serve as a drug screening array for the evaluation of drug response, customized to each patient individually by including patient-derived tumor, stromal and immune cells in the 3D-bio-ink. The 3D tumor model can be printed rapidly and robustly, allow testing several drugs or their combinations simultaneously with such perfusion system.

The present inventors also showed that the dormancy state of human cell line, thus far only observed in SCID mice and not in 2D culture, can be exhibited in the 3D-model described herein.

Furthermore, the present inventors demonstrated that the 3D-model can be used for a more accurate evaluation of response to therapies compared to 2D culture methods. Inhibition of P-Selectin, overexpressed in GB tumors, by SELPi did not have any effect on cells grown in 2D culture, but resulted in a remarkable reduction in GB cell proliferation in the fibrin 3D-bio-ink. These differences can be attributed to the elevated expression levels of P-Selectin by GB cells grown in fibrin 3D-model and in-vivo compared to cells grown in 2D cultures. Using embodiments of the present platform, it is possible to screen traditional chemotherapeutics, biological treatments, and immunotherapies as well as target adhesion molecules. These evaluations provide additional evidence that conventional 2D culture strategies relying on monolayers of cancer cells may not be sufficient to capture the complex TME.

Gene expression profiling showed that the transcriptional signature of murine GB cells grown in fibrin 3D-bio-ink are more similar to the cells grown orthotopically in mice than to cells grown in 2D culture. The analysis highlighted several genes that were similarly upregulated in cells grown in 3D-bio-ink and in-vivo compared to cells grown in 2D culture, among them several oncogenes and prognosis biomarkers for GB patient survival.

Regarding the standard of care for GB, the present inventors have evaluated the response of 3 patient-derived cells to treatment with TMZ. The results showed different responses to TMZ between the different patient-derived cells when grown in the 3D models, while there were no differences observed in the response to TMZ when the cells were grown as 2D models. This demonstrates that the 3D model reflects the broad spectrum of drug response seen in patients better than the traditional 2D models, that resulted in similar responses for TMZ on different origin of GB cells from several patients. In addition, patient-derived cells grown in the fibrin 3D-bio-ink tumor models were more resistant to TMZ treatment with diverse IC₅₀ values in μM scale (1000, 1400, 280 μM), compared to cells grown in 2D culture with an IC₅₀ value in nM scale (all around 0.004 μM).

In summary, using human and murine GB models or brain metastasis, the present inventors show herein that the perfusable 3D-bioprinted platform can serve as a reliable alternative pre-clinical tool.

Thus, according to an aspect of the invention there is provided a three dimensional (3D) bioprinted model of a glioblastoma (GB) tumor or a brain metastasis comprising a first hardened synthetic material and a plurality of cell types of said tumor or said brain metastasis and microenvironment thereof, said plurality of cell types comprising endothelial cells, pericytes, astrocytes, microglia cells and GB tumor cells or metastatic cells.

Herein throughout, in the context of bioprinting, the term “three dimensional (3D) bioprinted model” is also referred to herein interchangeably as “object”, “model” or “model object” or “bioprinted object” or “bioprinted model” refers to an engineered 3D model of a tumor (e.g., 3D-bioprinted tumor model or 3D-bioprinted model of a tumor), whereby the tumor model comprises malignant cells, and non-malignant cells, i.c., stroma and vasculature such that the tumor model represents also the tumor microenvironment.

The term “object” as used herein throughout refers to a whole object or a part thereof.

In the context of the present embodiments, the term “object” Accordingly, in some embodiments, the term “object” describes a region of interest (ROI) which comprises a tumor and optionally and preferably also the tumor's microenvironment (the microenvironment that surrounds the tumor).

As used herein “glioblastoma”, abbreviated as GB and previously termed glioblastoma multiforme and abbreviated as GBM refers to a highly aggressive tumor found in the brain and/or spinal cord. The following provides a summary of some types of GB envisaged according to some embodiments of the present invention. The classification can be made based on molecular markers, stem cells, metabolics and more, some of which is summarized infra.

### Molecular Markers

Four subtypes of glioblastoma have been identified based on gene expression:

Classical: Around 97% of tumors in this subtype carry extra copies of the epidermal growth factor receptor (EGFR) gene, and most have higher than normal expression of EGFR, whereas the gene TP53 (p53), which is often mutated in glioblastoma, is rarely mutated in this subtype. Loss of heterozygosity in chromosome 10 is also frequently seen in the classical subtype alongside chromosome 7 amplification.

The proneural subtype often has high rates of alterations in TP53 (p53), and in PDGFRA, the gene encoding a-type platelet-derived growth factor receptor, and in IDHI, the gene encoding isocitrate dehydrogenase-1.

The mesenchymal subtype is characterized by high rates of mutations or other alterations in NF1, the gene encoding neurofibromin 1 and fewer alterations in the EGFR gene and less expression of EGFR than other types.

The neural subtype is typified by the expression of neuron markers such as NEFL, GABRA1, SYT1, and SLC12A5, while often presenting themselves as normal cells upon pathological assessment.

Many other genetic alterations have been described in glioblastoma, and the majority of them are clustered in two pathways, the RB and the PI3K/AKT. Glioblastomas have alterations in 68-78% and 88% of these pathways, respectively.

Another important alteration is methylation of MGMT, a “suicide” DNA repair enzyme. Methylation impairs DNA transcription and expression of the MGMT gene. Since the MGMT enzyme can repair only one DNA alkylation due to its suicide repair mechanism, reserve capacity is low and methylation of the MGMT gene promoter greatly affects DNA-repair capacity. MGMT methylation is associated with an improved response to treatment with DNA-damaging chemotherapeutics, such as temozolomide.

### Cancer Stem Cells

Glioblastoma cells with properties similar to progenitor cells (glioblastoma cancer stem cells) have been found in glioblastomas. Glioblastoma cancer stem cells share some resemblance with neural progenitor cells, both expressing the surface receptor CD133. CD44 can also be used as a cancer stem cell marker in a subset of glioblastoma tumor cells.

### Metabolism

The IDH1 gene encodes for the enzyme isocitrate dehydrogenase 1 and is uncommonly mutated in glioblastoma (primary GBM: 5%, secondary GBM>80%).

### Ion Channels

GB exhibits numerous alterations in genes that encode for ion channels, including upregulation of gBK potassium channels and CIC-3 chloride channels.

### Tumor Vasculature

GBM is characterized by abnormal vessels that present disrupted morphology and functionality. The high permeability and poor perfusion of the vasculature result in a disorganized blood flow within the tumor and can lead to increased hypoxia, which in turn facilitates cancer progression by promoting processes such as immunosuppression.

According to a specific embodiment, the term relates also to recurrent glioblastoma.

As used herein “brain metastasis cell” refers to a cancer cell that metastasized to the brain however its primary origin is not brain. Metastatic brain tumors (i.e., cancer that began somewhere else in the body spreads to the brain and causes a mass or brain tumor) include, but are not limited to, lung, breast, melanoma, colon, kidney and thyroid gland cancers.

GB cells or brain metastasis cells are collectively referred to herein as the “tumor cells”. Examples of GB cell lines include but are not limited to U-87MG, T98G and U373. Microglial cells and astrocytes are collectively referred to herein as “stromal cells” or “stroma”. Other stromal cells which can be included for example are neurons, oligodendroglia, immune cells/PBMC and mesenchymal stem cells. The stromal and vascular cells are typically non-cancerous.

As used herein “astrocytes” refers to glial cells typically expressing key proteins including glial fibrillar acidic protein (GFAP), glutamate transporter (Glt1/EAAT2) and the gap junction protein connexin 30 (Cx30). Particularly, GFAP expression, often used as a reliable astrocyte marker. Markers for mature astrocytes include, but are not limited to, aldehyde dehydrogenase family 1 member L1 (Aldh1L1), aldolase C (AldoC), glutamate transporter-1 (Glt1), S100 calcium-binding protein B (S100b) and Aquaporin 4.

As used herein “reactive astrocytes” refer to astrocytes responding to abnormal events in the CNS, including neurodegenerative and demyelinating diseases, epilepsy, trauma, ischemia, infection, and particularly cancer. Markers include, but are not limited to GFAP, β-catenin, nestin, and N-cadherin, as can be analyzed at the RNA level such as by RT-PCR or at the protein level such as by immunostaining, FACS or Western blot (relevant to other markers described herein too). Escartin et al. Nat Neurosci. 2021 Mar; 24(3): 312-325. describes the various methods for identifying astrocytes and reactive astrocytes and is hereby incorporated by reference in its entirety.

Examples of normal human astrocyte cell lines include but are not limited to human primary astrocytes (HA, Catalog #1800, ScienCell).

As used herein “microglia” refer to a type of neuroglia (glial cell) located throughout the brain and spinal cord. Microglia account for 10-15% of all cells found within the brain. As the resident macrophage cells, they act as the first and main form of active immune defense in the central nervous system (CNS). Microglial share a similar sensome to other macrophages, however they contain 22 unique genes, 16 of which are used for interaction with endogenous ligands. These differences create a unique microglial biomarker that includes over 40 genes including P2ryl2 and HEXB. DAP12 (TYROBP). Potentially useful markers of microglia are described in Hickman SE, Kingery ND, Ohsumi TK, Borowsky ML, Wang LC, Means TK, El Khoury J (December 2013). “The microglial sensome revealed by direct RNA sequencing”. Nature Neuroscience. 16 (12): 1896-1905, which is hereby incorporated by reference in its entirety.

Examples of normal human microglia cell lines include but are not limited to HML3 (CRL-3304, ATCC) and HM, Catalog #1900, ScienCell.

As used herein “activated” microglia or “reactive” microglia refers to microglial cells which become activated following exposure to pathogen-associated molecular patterns (PAMPs) and/or endogenous damage-associated molecular patterns (DAMPs), and removal of the immune-suppressive signals. Activated microglia can acquire different phenotypes depending on cues in their surrounding environment. Typically expressing the Iba1 marker.

Pericytes and endothelial cells establish the vascularization in the 3D model according to some embodiments of the invention.

As used herein “pericytes” refer to cells present at intervals along the walls of capillaries (and post-capillary venules). In the central nervous system (CNS), they are important for blood vessel formation, maintenance of the blood-brain barrier, regulation of immune cell entry to the central nervous system (CNS) and control of brain blood flow. Typical markers include, but are not limited to α-SMA and NG2.

Examples of normal human pericyte cell lines and primary cells include but are not limited to Human Brain Vascular Pericytes (HBVP) Catalog #1200, ScienCell.

The Examples section which follows provides examples of cell lines of pericytes, endothelial and microglial cell from commercial vendors such as the ATCC and ScienCell (California, USA).

As used herein “endothelial cells” to the cells which line the interior surface of the blood vessel. Endothelial cells release substances that control vascular relaxation and contraction as well as enzymes that control blood clotting, immune function and platelet (a colorless substance in the blood) adhesion. Typical markers include, but are not limited to, CD31/PECAM-1, Angiotensin-converting enzyme, Factor VIII-related antigen, Ulex europaeus I agglutinin binding/O(H) blood-type antigen, Vascular endothelial cadherin, CD36, CD105/endoglin, CD73 and AAMP.

Examples of human endothelial cells cell lines and primary cells include but are not limited to HAUC, HPAEC, HUVEC, HDMVEC, HAMEC, HCMEC/D3 and HBEC, all available from ATCC.

It will be appreciated that cells as described herein are commercially available or can be self-isolated.

It will be appreciated that the use of more than one marker and/or the use of negative markers may improve the specificity of identification of the desired cells. The use of more than one marker is also referred to as a “signature”.

According to a specific embodiment, the 3D model is devoid of smooth muscle cells and/or neurons.

According to a specific embodiment, the cells can be of primary cells, non-immortalized cells, freshly isolated from a patient without any culturing or cloning, cell lines or a combination of same.

According to a specific embodiment, the cells are mammalian cells e.g., mouse.

According to a specific embodiment, the cells are human cells.

According to some embodiment, the cells are from a single host (e.g., at least the tumor cells).

According to a specific embodiment, the cells are from different hosts.

According to a specific embodiment, the cells are from different hosts (e.g., different human beings) or organism origin e.g., human and mouse.

According to a specific embodiment, each of the plurality of cell types is from different organisms (e.g., different patients).

According to a specific embodiment, some of the plurality of cell types are from different organisms (e.g., different patients, e.g., the tumor cells and stroma are from one organism and the vascularization cells are from other(s)).

According to a specific embodiment, each of the plurality of cell types is from the same organism.

According to a specific embodiment, all the cells are autologous to the subject on which personalized screening for drugs will take place using the 3D model (e.g., full HLA matchability as described below).

As used herein “full match HLA” refers to 100% identical HLA alleles. Embodiments of the invention relate to 3D tumor models which comprise cells derived from a single donor.

According to some embodiments, these tumors are micro-engineered based on in vivo imaging data, and their 3D structure features a high match to their original architecture.

Such 3D tumor models may find various uses in drug screening and personalized therapy. For example, several drugs (as monotherapy or combination therapies) can be tested on them within days—a process that is useful in cases of aggressive tumors.

According to a specific embodiment, at least some of the cell types are comprise fully matched HLA.

Generally, in order to produce the model, the tumor cells and stroma (microglia+astrocytes) are retrieved and mixed with a first synthetic material forming the tumor bio-ink.

When obtained from tissue biopsies the tissue is mechanically and/or enzymatically processed to obtain viable single cells or aggregates thereof not exceeding 500 (e.g., 250, 100, 50 or 10) cells/aggregate. An exemplary protocol for isolation is provided in the Examples section which follows. Typically, a tumor biopsy is obtained. The tissue is processed to obtain cells in suspension which are selected by marker expression. Vascular forming cells such as the pericytes and endothelial cells are isolated and the rest of the cells are taken to form the tumor bio-ink. Of note, the tumor bio-ink may comprise the tumor cells, microglia and astrocytes but other cells can be present too in this suspension.

An embodiment of the protocol is provided in the Examples section which follows relating to stromal cells isolation.

According to some embodiments, the cells or parts of the cell types are labeled.

Methods of labeling cells and useful dyes and genetic dyes are well known in the art.

Cells, cell clusters, organelles visualization with selective stains or dyes or fluorescent proteins are key tools in fluorescence imaging of cells and tissues. These specific stains are suitable counterstains to antibodies to help the identification of location-specific targets of interest within the cell. Dyes for live cell staining of organelles are available in a broad spectrum of colors. Such labels are known in the art.

Examples include but are not limited to those listed in Salipalli et al. 2014 BMC Cell Biol. 15: 26, which is hereby incorporated by reference in its entirety.

Measures are taken for using different labels for different cell types according to the intended use.

According to other embodiments, the cells or parts pf the cell types are unlabeled.

Mechanical, physical, biological and chemical properties of the model described herein are further described hereinbelow following description of the method which is used to produce the model of the present invention.

Thus, according to an aspect of the invention, there is provided a method of producing the 3D model comprising:
(a) dispensing at least one layer of astrocytes, microglia cells and GB tumor cells or metastatic cells comprised in a first synthetic material;
(b) forming a vascularization pattern on said at least one layer using a second synthetic material which is liquefiable to allow formation of perfusable tubular vessels comprising lumen; wherein step (a) is performed prior to and following step (b);
(c) dispensing said second synthetic material to form said lumen;
(d) applying endothelial cells and pericytes into said lumen to form blood vessels.

As used herein in the context of the present embodiments, the term “dispensing” refers to depositing a material in a configured pattern that corresponds to a desired shape of respective three-dimensional object or a part thereof. According to some embodiments, dispensing can be performed digitally, for example, via any of the known additive manufacturing methodologies as described herein, e.g., printing, bioprinting, or via non-digital methodologies such as, for example, casting (e.g., mold casting).

As used herein in the context of the present embodiments, the term “casting” refers to a methodology in which a liquid material or formulation is placed in a mold and allows to harden, to thereby form an object, or a part thereof, in a shape of the mold.

As used herein, “bioprinting” or it's shortened version “printing” means practicing an additive manufacturing process, preferably a 3D-inkjet printing process or extrusion printing, while utilizing one or more bio-ink formulation(s) that comprises cells or cellular components (e.g., cell solutions, cell-containing gels, cell suspensions, cell concentrations, multicellular aggregates, multicellular bodies, etc.) via methodology that is compatible with an automated or semi-automated, computer-aided, additive manufacturing system as described herein (e.g., a bioprinter or a bioprinting system).

### Additive Manufacturing:

According to some embodiments of this aspect, the method is effected by sequentially forming a plurality of layers in a configured pattern corresponding to the shape of the object, thereby forming the object. According to some embodiments of this aspect, formation of each layer is effected by dispensing at least one uncured building material, and exposing the dispensed building material to a curing condition to thereby form a hardened (cured) material.

Herein throughout, the phrase “uncured building material” or “uncured building material formulation” collectively describes the materials that are used to sequentially form the layers, as described herein. This phrase encompasses uncured materials which form the final object, namely, one or more uncured modeling material formulation(s), and optionally also uncured materials used to form a support, namely uncured support material formulations. According to some embodiments, this refers to the first synthetic material and/or second synthetic material in their curable/uncured form.

An uncured building material can comprise one or more modeling formulations, and can be dispensed such that different parts of the object are made upon curing different modeling formulations, and hence are made of different cured modeling materials or different mixtures of cured modeling materials.

The method of the present embodiments manufactures three-dimensional objects in a layer-wise manner by forming a plurality of layers in a configured pattern corresponding to the shape of the object.

Each layer is formed by an additive manufacturing apparatus which scans a two-dimensional surface and patterns it. While scanning, the apparatus visits a plurality of target locations on the two-dimensional layer or surface, and decides, according to a pre-set algorithm, for each target location or a group of target locations, whether or not the target location or group of target locations is to be occupied by a building material, and which type of a building material is to be delivered thereto. The decision is made according to a computer image of the surface.

When the AM is by three-dimensional inkjet printing, an uncured building material, as defined herein, is dispensed from a dispensing head having a set of nozzles to deposit building material in layers on a supporting structure. The AM apparatus thus dispenses building material in target locations which are to be occupied and leaves other target locations void. The apparatus typically includes a plurality of dispensing heads, each of which can be configured to dispense a different building material (for example, different modeling material formulations, each containing a different cell type). Thus, different target locations can be occupied by different building materials (e.g., a modeling formulation and/or a support formulation, as defined herein).

The final three-dimensional object is made of the modeling material or a combination of modeling materials or a combination of modeling material/s and support material/s or modification thereof (e.g., following curing). All these operations are well-known to those skilled in the art of additive manufacturing (also known as solid freeform fabrication).

In some exemplary embodiments of the invention, an object is manufactured by dispensing a building material that comprises two or more different modeling material formulations, each modeling material formulation from a different dispensing head of the AM apparatus. The modeling material formulations are, optionally and preferably, deposited in layers during the same pass of the printing heads. The modeling material formulations and/or combination of formulations within the layer are selected according to the desired properties of the object.

An exemplary 3D printing method according to some embodiments of the present invention starts by receiving 3D printing data corresponding to the shape of the object. The data can be received, for example, from a host computer which transmits digital data pertaining to fabrication instructions based on computer object data, e.g., in a form of a Standard Tessellation Language (STL) or a StereoLithography Contour (SLC) format, Virtual Reality Modeling Language (VRML), Additive Manufacturing File (AMF) format, Drawing Exchange Format (DXF), Polygon File Format (PLY), Digital Imaging and Communications in Medicine (DICOM) or any other format suitable for Computer-Aided Design (CAD).

The method continues by dispensing droplets of the uncured building material as described herein in layers, on a receiving medium, using one or more printing heads, according to the printing data. The receiving medium can be a tray of a printing system or a previously deposited layer.

Once the uncured building material is dispensed on the receiving medium according to the 3D printing data, the method optionally and preferably continues by exposing the deposited layers to a curing condition. Preferably, the curing condition is applied to each individual layer following the deposition of the layer and prior to the deposition of the previous layer.

Exposure to a curing condition is typically performed using a curing energy source which can be, for example, a radiation source, such as an ultraviolet or visible or infrared lamp, or other sources of electromagnetic radiation, or electron beam source, depending on the modeling material formulation(s) being used. The curing energy source serves for curing or solidifying (hardening) at least the modeling material formulation(s). Alternatively, a curing condition can include a presence of a chemical or biological reagent that promotes curing.

Some AM processes according to the present embodiments involve dispensing materials (e.g., hydrogels, for example, Pluronic hydrogels) without exposing these materials to curing energy but rather to a curing condition as defined herein. Such hydrogels can harden, for example, in the presence of calcium ions or when a formulation containing same is cooled.

Some embodiments contemplate the fabrication of an object by dispensing different formulations from different dispensing heads. These embodiments provide, inter alia, the ability to select formulations from a given number of formulations and define desired combinations of the selected formulations and their properties. According to the present embodiments, the spatial locations of the deposition of each formulation with the layer are defined, either to effect occupation of different three-dimensional spatial locations by different formulations, or to effect occupation of substantially the same three-dimensional location or adjacent three-dimensional locations by two or more different formulations so as to allow post deposition spatial combination of the formulations within the layer.

The present embodiments thus enable the deposition of a broad range of material combinations, and the fabrication of an object which may consist of multiple different combinations of modeling material formulations, in different parts of the object, according to the properties desired to characterize each part of the object.

A printing system utilized in additive manufacturing may include a receiving medium and one or more printing heads. The receiving medium can be, for example, a fabrication tray that may include a horizontal surface to carry the material dispensed from the printing head. The printing head may be, for example, an ink jet head having a plurality of dispensing nozzles arranged in an array of one or more rows along the longitudinal axis of the printing head. The printing head may be located such that its longitudinal axis is substantially parallel to the indexing direction. The printing system may further include a controller, such as a microprocessor to control the printing process, including the movement of the printing head according to a pre-defined scanning plan (e.g., a CAD configuration converted to a Standard Tessellation Language (STL) format and programmed into the controller). The printing head may include a plurality of jetting nozzles. The jetting nozzles dispense material onto the receiving medium to create the layers representing cross sections of a 3D object.

In addition to the printing head, there may be a source of curing energy, for curing the dispensed building material. The curing energy is typically radiation, for example, UV radiation or heat radiation. Alternatively, there may be means for providing a curing condition other than electromagnetic or heat radiation, for example, means for cooling the dispensed building material of for contacting it with a reagent that promotes curing.

Additionally, the printing system may include a leveling device for leveling and/or establishing the height of each layer after deposition and at least partial solidification, prior to the deposition of a subsequent layer.

According to the present embodiments, the additive manufacturing method described herein is for bioprinting a biological object.

Herein throughout, the phrase “modeling material formulation”, which is also referred to herein interchangeably as “modeling formulation” or “modeling material composition” or “modeling composition”, describes a part or all of the uncured building material which is dispensed so as to form the object, as described herein. The modeling formulation is an uncured modeling formulation, which, upon exposure to a curing condition, forms the object or a part thereof.

In the context of bioprinting, an uncured building material comprises at least one modeling formulation that comprises one or more cells (e.g., tumor cells and microenvironment thereof, as defined herein, e.g., astrocytes and microglia) as described herein, and is also referred to herein and in the art as “bio-ink” or “bio-ink formulation”.

In some embodiments, the bioprinting comprises sequential formation of a plurality of layers of the uncured building material in a configured pattern, preferably according to a three-dimensional printing data, as described herein. At least one, and preferably most or all, of the formed layers comprise(s) a cellular component, preferably a plurality of cellular components, as described herein. Optionally, at least one of the formed layers comprises one or more curable materials, preferably biocompatible curable materials which do not interfere with the biological and/or structural features of the cellular components in the bio-ink. According to some embodiments some of the modeling formulations do not comprise cells when printed.

As mentioned, printing at least one layer of astrocytes, microglia cells and GB tumor cells or metastatic cells comprised in a first synthetic material, is typically referred to as a first modeling formulation.

Printing a vascularization pattern on said at least one layer using a second synthetic material which is liquefiable to allow formation of perfusable tubular vessels comprising lumen, is typically referred to as a second modelling formulation.

In some embodiments, the one or more curable materials comprise a synthetic material being exogenous to the tumor or its surrounding environment (region of interest (ROI)). However, in this case, the second synthetic material, i.c., Pluronic is essentially depleted from the model by liquification/melting.

As used herein “exogenous” refers to a material that is non-naturally present in the tumor or its surrounding environment within the subject, and further encompasses a material that is not derived from the subject or is not inherently present in the subject.

Thus, the curable material comprises a synthetic material, or a material that forms a synthetic material upon exposure to a curing condition as described herein.

By “synthetic material” it is meant a material that is not inherently present in the tumor or its environment, or in the subject in general. This term encompasses materials that are obtained from a source that is other than the tumor and its environment, and optionally a source that is other than the subject afflicted with the tumor. This term encompasses biological and non-biological materials, naturally-occurring and non-naturally occurring materials, and synthetically prepared materials.

For example, the cross linked fibrin/gelatin formulation in the presence of thrombin using an enzymatic cross-linker, e.g., TG is a synthetic material which is not found in the body.

In some embodiments, a three-dimensional printing data that is readable by the bioprinting system is generated based on a three-dimensional imaging data, as described herein.

According to an aspect of some embodiments of the present invention the 3D model is designed according to imaging data of the natural tumor. Thus according to some embodiments, the method comprises imaging the tumor to acquire a 3D model of the tumor and optionally a surrounding environment of the tumor, that is, for example, employing a three-dimensional medical imaging technique to thereby acquire a three-dimensional imaging data of the tumor and optionally its surrounding environment (ROI);
ex-vivo dissociating the tumor and optionally its surrounding environment so as to obtain a cell suspension comprising a plurality of cell types; and subjecting the cell suspension to bioprinting according to the 3D model (or 3D imaging data) so as to obtain a 3D model of the tumor (and optionally its surrounding environment). The latter step and optionally the dissociation step are present regardless of whether the method is based on 3D imaging of the natural tumor.

In some embodiments, the bioprinting comprises receiving 3D printing data and forming the layers in accordance with the 3D printing data, whereby the 3D printing data is generated based on the 3D imaging data. Thus, the 3D model of the tumor features a 3D arrangement (structure, architecture) that has at least 70%, at least 80%, at least 90%, at least 95%, at least 98%, at least 99% or higher, match with the 3D imaging data.

Determining a match to the 3D imaging data can be made by determining the % of voxels in the bioprinted tumor that are identical to voxels of the 3D imaging data and/or comparing other coordinates or parameters of the bioprinted tumor model to corresponding coordinates and/or parameters of the 3D imaging data.

Alternatively, or in addition, the matchability to the 3D imaging data can be determined by the quality of the polymeric scaffold and its ability to mimic the anatomical structure of the tumor. The parameters tested for validation include, for example, swelling capabilities, elasticity, mechanical strength, porosity, etc. Methods of determining such parameters are well-known to those skilled in the art and some exemplary methods are described in the Examples section that follows.

In some embodiments, imaging the tumor is effected using a medical imaging technique as described herein. The imaging can be effected in vivo or ex vivo (upon dissecting the tumor or a portion thereof).

The bioprinting method described herein meets an essential requirement for reproducing the complex, heterogeneous architecture of the tumor upon a comprehensive understanding of the composition and organization of its components. This is achieved by utilizing medical imaging technologies/techniques which can provide the required information on 3D structure and function at the cellular, tissue, organ and organism levels. These technologies include most noninvasive imaging modalities, the common being computed tomography (CT), or μCT, and magnetic resonance imaging (MRI), or μMRI, though other imaging technologies can be used e.g., ultrasound, X-ray. Computer-aided design and computer-aided manufacturing (CAD-CAM) tools and mathematical modeling are also used to collect and digitize the complex tomographic and architectural information for tissues (3). For example, MRI/CT imaging is used to acquire an accurate digital 3D model of the region of interest (ROI) of the patient's tumor and its surrounding microenvironment. MRI provides high spatial resolution in soft tissue, with the advantage of increased contrast resolution, which is useful for imaging soft tissues in close proximity to each other.

In some embodiments, at least a portion of the tumor is removed from a subject and is thereafter dissociated, such that the method comprises, prior to dissociating the tumor, removing a portion of the tumor, and a surrounding environment of the tumor, from a subject. This can be done by means of a surgery, a biopsy, and any other acceptable means. Obtaining the 3D imaging data can be made prior to or subsequent to removing the tumor or a portion thereof.

In some embodiments, dissociating the tumor (or a portion thereof and/or a surrounding environment thereof) is effected by enzymatic dissociation and/or mechanical dissociation.

The obtained cell suspension is then used as a bio-ink or a part thereof as described herein in a selected bioprinting method and a corresponding bioprinting system, for example, as described herein, in combination with one or more acellular curable materials, for example as described herein.

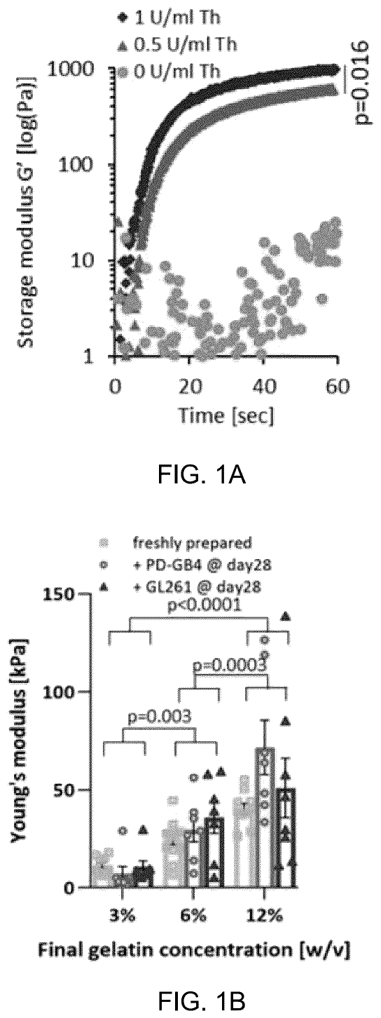

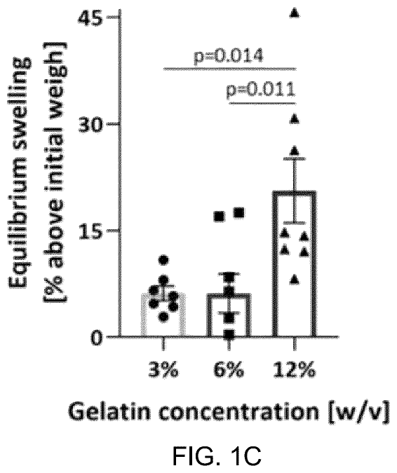

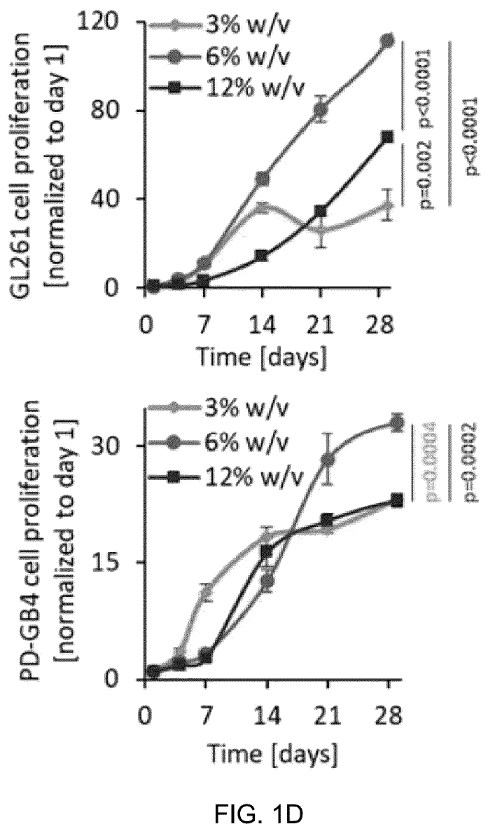

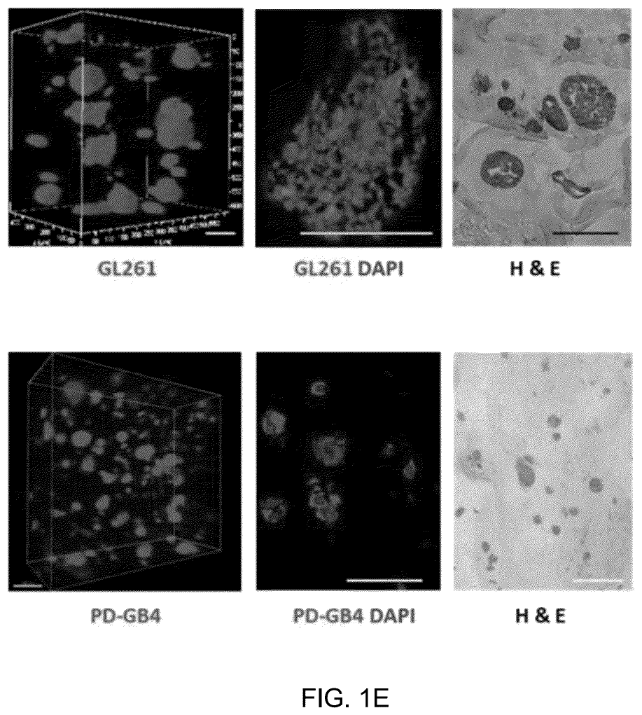

In some embodiments of the present invention, from each tumor sample collected, e.g., during surgery, the tumor is partially recreated by 3D bioprinting as presented herein, and, in addition, cells are isolated for tissue culture, a piece of the tumor is implanted in SCID mice as patient-derived xenograft (PDX), and/or formalin-fixed paraffin-embedded (FFPE) slides are created for histology (as shown in FIGS. 1A-E). All these models (a combination of a bioprinted model of the tumor and one or more of the above-mentioned and optionally other models) can provide a picture that better mimics the clinical setting.

In some embodiments, the bioprinting comprises transferring the obtained 3D imaging data to a 3D printing data readable by a bioprinting system usable in the bioprinting.

In some embodiments, the bioprinting comprises sequentially forming a plurality of layers on a receiving medium in a configured pattern corresponding to said 3D printing data, such that at least one of the layers comprises cells of the cell suspension.

In some embodiments, at least one of the layers comprises a synthetic curable material, or a curable material that forms a synthetic material, as described herein, upon exposure to a curing condition as described herein.

In some embodiments, the curable material is an acellular curable material.

In some embodiments, the curable (e.g., synthetic) material and the 3D printing data are selected or designed so as to provide a chemical, physical and/or mechanical property to the 3D tumor model. In some embodiments, the bioprinting further comprises exposing at least one layer which comprises the curable material to a curing condition (e.g., curing energy), to thereby provide a hardened synthetic (e.g., exogenous and/or acellular, as defined herein) material.

In some embodiments, the hardened synthetic material provides a chemical, physical and/or mechanical property to the 3D tumor model.

In some embodiments, the curable material (e.g., which provides a hardened synthetic material, preferably an exogenous material) and the 3D printing data are selected so as to provide a chemical, physical and/or mechanical property at a pre-determined target location in the 3D tumor model, in accordance with the printing data.

In some embodiments, a method as described herein further comprises characterizing the obtained tumor model, for example, by isolating cells of the tumor model; and in vitro or in vivo culturing the cells.

In some of any of the embodiments described herein, the bioprinting method is configured to effect formation of the layers under conditions that do not significantly affect structural and/or functional properties of the cellular components in the bio-ink. In some embodiments, a bioprinting system for effecting a bioprinting process/method as described herein is configured so as to allow formation of the layers under conditions that do not significantly affect structural and/or functional properties of the cellular components in the bio-ink.

In some embodiments, the acellular curable materials and/or the curing condition applied to effect curing are selected such that they do not significantly affect structural and/or functional properties of the cellular components in the bio-ink.

### Bioprinting Techniques:

A bioprinting method and a corresponding system can be any of the methods and systems known in the art for performing additive manufacturing, and exemplary such systems and methods are described hereinabove. A suitable method and system can be selected upon considering its printing capabilities, which include resolution, deposition speed, scalability, bio-ink compatibility and case-of-use.

Exemplary suitable bioprinting systems usually contain a temperature-controlled material handling with a dispensing system and stage (a receiving medium), and a movement along the x, y and z axes directed by a CAD-CAM software. A curing source (e.g., a light or heat source) which applies a curing energy (e.g., by applying light or heat radiation) or a curing condition to the deposition area (the receiving medium) so as to promote curing of the formed layers and/or a humidifier, can also be included in the system. There are printers that use multiple dispensing heads to facilitate a serial dispensing of several materials.

In some embodiments, the printing provides a printed tumor featuring a plurality of voxel blocks, and at least 70%, at least 80%, at least 90%, or more, as described herein, of these voxel blocks are identical to corresponding voxel blocks of the 3D imaging data used for generating the 3D printing data.

Generally, bioprinting can be effected using any of the known techniques for additive manufacturing. The following lists some exemplary additive manufacturing techniques, although any other technique is contemplated.

### 3D Inkjet Printing:

3D Inkjet printing is the most commonly used type of 3D printer for both non-biological and biological (bioprinting) applications. Inkjet printers use thermal or acoustic forces to eject drops of liquid onto a substrate, which can support or form part of the final construct. In this technique, controlled volumes of liquid are delivered to predefined locations, and a high-resolution printing with precise control of (1) ink drops position, and (2) ink volume, which is beneficial in cases of microstructure-printing or when small amounts of bioreactive agents or drugs are added, is received (7). Inkjet printers can be used with several types of ink i.c., to use multiple types of cells and ECMs as well as multiple bioactive agents. Furthermore, the printing is fast and can be applied onto culture plates.

A bioprinting method that utilizes a 3D inkjet printing system can be operated using one or more bio-ink modeling material formulations as described herein, and dispensing droplets of the formulation(s) in layers, on the receiving medium, using one or more inkjet printing head(s), according to the 3D printing data.

### Extrusion Printing:

This technique uses continuous beads of material rather than liquid droplets. These beads of material are deposited in 2D, the stage (receiving medium) or extrusion head moves along the z axis, and the deposited layer serves as the basis for the next layer. The most common methods for biological materials extrusion for 3D bioprinting applications are pneumatic (8, 9) or mechanical (10, 11) dispensing systems. The main advantage of this technique is the ability to deposit very high cell densities. Extrusion bioprinters have been used to construct multiple tissue types, amongst them aortic valves and branched vascular trees as well as for in-vitro pharmacokinetic profiles and tumor modeling (3). The downside of extrusion bioprinting is that fabrication time is relatively slow when printing high-resolution complexed structures.

### Laser-Assisted Printing:

Laser-assisted printing (also known as laser-assisted stereolithography) technique is based on the principle of laser-induced forward transfer, which was developed to transfer metals and is now successfully applied to biological materials. The device consists of a laser beam, a focusing system, a ribbon that has a donor transport support (usually made of glass) that is covered with a laser energy absorbing layer (e.g., gold or titanium), a biological material layer (e.g., cells and/or hydrogel) and a receiving substrate facing the ribbon (3). A laser assisted printer operates by shooting a laser or a binding material at a bed of powder and solidifying it in a highly specific pattern. As the laser or binding agent moves through the powder, layer by layer, it builds a solid structure embedded in powder, which is dusted off when the job is done (3).

Laser associated printing is compatible with a series of viscosities and can print mammalian cells without affecting cell viability or cell function. Cells can be deposit at a density of up to 10⁸ cells/ml with microscale resolution of a single cell per drop (12, 13).

### Electrospinning:

Electrospinning is a fiber production technique, which uses electric force to draw charged threads of polymer solutions, or polymer melts. This cell-laden printing could provide an approach to create small diameter capillary-like blood vessels (14). Another printing technique uses a supporting bath which contains sacrificial hydrogel as a thermoreversible mold to embed the printing of the desired structure from another hydrogel (15). The supporting bath can be made of the Pluronic family of hydrogels or Gelatin.

In some of any of the embodiments described herein, the bioprinting comprises, or consists of, 3D-inkjet printing, as is well-known in the art and is described herein.

Additional bioprinting methodologies include, for example, SWIFT (sacrificial writing into functional tissue), that allows the 3D printing of vascular channels into living matrices composed of organ building blocks (OBBs), as described, for example, in Cartola V. Harvard researchers develop new technique to create human organs. 3D Natives. 2019; Skylar-Scott et al. Sci. Adv. 2019; 5: eaaw2459; and Uijung Yong et al. Prog. Biomed. Eng. 2020: 2: 042003; and freeform reversible embedding of suspended hydrogels (known as FRESH), which uses a thermoreversible support bath to enable deposition of hydrogels in complex, 3D biological structures and involves deposition and embedding of the hydrogel(s) being printed within a second hydrogel support bath that maintains the intended structure during the print process and significantly improves print fidelity, such as described, for example, in Hinton et al., Sci. Adv. 1(9). e1500758 (2015); Lee et al., Science 365(6452), 482-487 (2019); Bhattacharjee et al., Sci. Adv. 1(8), e1500655 (2015); and Shiwarski et al., APL Bioeng. 2021 Mar; 5(1): 010904.

Any other bioprinting methodology is contemplated in the context of the present embodiments.

### Modeling Material Formulations (Bio-Ink):

According to some embodiments of the invention, the modeling formulations as used herein are distinct from one another. The first comprises a first synthetic material which serves as the tumor bio-ink, in this case, a combination of fibrinogen, gelatin, thrombin and Transglutaminase (TG) to which the tumor and stroma cells are added. The second comprises Pluronic e.g., F127 and optionally thrombin and serves as the vascular bio-ink.

Thus, at least in the case of the tumor bio-ink, according to some of the present embodiments, it comprises a cell suspension comprising a plurality of cellular components.

In some embodiments, the bio-ink further comprises one or more curable materials e.g., gelatin and fibrinogen.

In some embodiments, the curable material is, or is selected so as to form, a synthetic material as defined herein (e.g., acellular; exogenous; non-biological material as defined herein).

In some embodiments, the bio-ink further comprises a non-cellular (acellular) curable material that forms, upon curing, a synthetic (e.g., exogenous) material as defined herein.

Herein throughout, a “curable material” is a compound (monomeric or oligomeric or polymeric compound) which, when exposed to a curing condition, as described herein, solidifies or hardens to form a cured modeling material as defined herein. Curable materials are typically polymerizable materials, which undergo polymerization and/or cross-linking when exposed to a suitable curing condition or a suitable curing energy (a suitable energy source). Alternatively, curable materials are thermo-responsive materials, which solidify or harden upon exposure to a temperature change (e.g., heating or cooling). Optionally, curable materials are made of small particles (e.g., nanoparticles or nanoclays) which can undergo curing to form a hardened material. Further optionally, curable materials are biological materials which undergo a reaction to form a hardened or solid material upon a biological reaction (e.g., an enzymatically-catalyzed reaction).

In some embodiments, a “curing condition” encompasses a curing energy (e.g., temperature, radiation) and/or a material or reagent that promotes curing.

In some of any of the embodiments described herein, a curable material is a photopolymerizable material, which polymerizes or undergoes cross-linking upon exposure to radiation, as described herein, and in some embodiments the curable material is a UV-curable material, which polymerizes or undergoes cross-linking upon exposure to UV-vis radiation, as described herein.

In some of any of the embodiments described herein, a curable material can be a monomer, an oligomer or a short-chain polymer, each being polymerizable as described herein.

In some of any of the embodiments described herein, when a curable material is exposed to a curing condition (e.g., radiation, reagent), it polymerizes by any one, or combination, of chain elongation or entanglements and cross-linking. The cross-linking can be chemical and/or physical.

In some of any of the embodiments described herein, a curable material is a monomer or a mixture of monomers which can form a polymeric modeling material upon a polymerization reaction, when exposed to a curing condition at which the polymerization reaction occurs. Such curable materials are also referred to herein as monomeric curable materials.

In some of any of the embodiments described herein, a curable material is an oligomer or a mixture of oligomers which can form a polymeric modeling material upon a polymerization reaction, when exposed to a curing condition at which the polymerization reaction occurs. Such curable materials are also referred to herein as oligomeric curable materials.

In some of any of the embodiments described herein, a curable material is or comprises a hydrogel, as defined herein, which can form a hardened modeling material, typically upon further cross-linking and/or co-polymerization, when exposed to a curing condition at which the cross-linking and/or co-polymerization reaction occurs. Such curable materials are also referred to herein as hydrogel curable materials.

In some of any of the embodiments described herein, a curable material is or comprises a hydrogel forming material, as defined herein, which can form a hydrogel as a hardened modeling material, typically upon cross-linking, polymerization and/or co-polymerization, when exposed to a curing condition at which the cross-linking, polymerization and/or co-polymerization reaction occurs. Such curable materials are also referred to herein as hydrogel-forming curable materials.

Herein and in the art, the term “hydrogel” describes a three-dimensional fibrous network containing at least 20%, typically at least 50%, or at least 80%, and up to about 99.99% (by mass) water. A hydrogel can be regarded as a material which is mostly water, yet behaves like a solid or semi-solid due to a three-dimensional crosslinked solid-like network, made of natural and/or synthetic polymeric chains, within the liquid dispersing medium. According to some embodiments of the present invention, a hydrogel may contain polymeric chains of various lengths and chemical compositions, depending on the precursors used for preparing it. The polymeric chains can be made of monomers, oligomers, block-polymeric units, which are inter-connected (crosslinked) by chemical bonds (covalent, hydrogen and ionic/complex/metallic bonds, typically covalent bonds). The network-forming material comprises either small aggregating molecules, particles, or polymers that form extended elongated structures with interconnections (the crosslinks) between the segments. The crosslinks can be in the form of covalent bonds, coordinative, electrostatic, hydrophobic, or dipole-dipole interactions or chain entanglements between the network segments. In the context of the present embodiments, the polymeric chains are preferably hydrophilic in nature.

The hydrogel, according to embodiments of the present invention, can be of biological origin or synthetically prepared.

According to some embodiments of the present invention, the hydrogel is biocompatible, and is such that when a biological moiety is impregnated or accumulated therein, an activity of the biological moiety is maintained, that is, a change in an activity of the biological moiety is no more than 30%, or no more than 20%, or no more than 10%, compared to an activity of the biological moiety in a physiological medium.

Exemplary polymers or co-polymers usable for forming a hydrogel according to the present embodiments include polyacrylates, polymethacrylates, polyacrylamides, polymethacrylamides, polyvinylpyrrolidone and copolymers of any of the foregoing. Other examples include polyethers, polyurethanes, and poly(ethylene glycol), functionalized by cross-linking groups or usable in combination with compatible cross linking agents.

Some specific, non-limiting examples, include: poly(2-vinylpiridine), poly(acrylic acid), poly(methacrylic acid), poly(N-isopropylacrylamide), poly(N,N′-methylenbisacrylamide), poly(N-(N-propyl)acrylamide), poly(methacyclic acid), poly(2-hydroxyacrylamide), poly (ethylene glycol) acrylate, poly (ethylene glycol) methacrylate, and polysaccharides such as dextran, alginate, agarose, and the like, and any co-polymer of the foregoing.

Hydrogel precursors (hydrogel-forming materials) forming such polymeric chains are contemplated, including any combination thereof.

Hydrogels are typically formed of, or are formed in the presence of, di- or tri- or multi-functional monomers, oligomer or polymers, which are collectively referred to as hydrogel precursors or hydrogel-forming agents or hydrogen-forming materials, having two, three or more polymerizable groups. The presence of more than one polymerizable group renders such precursors cross-linkable, and allow the formation of the three-dimensional network.

Exemplary cross-linkable monomers include, without limitation, the family of di- and triacrylates monomers, which have two or three polymerizable functionalities, one of which can be regarded as a cross-linkable functional group. Exemplary diacrylates monomers include, without limitation, methylene diacrylate, and the family of poly(ethylene glycol)n dimethacrylate (nEGDMA). Exemplary triacrylates monomers include, without limitation, trimethylolpropane triacrylate, pentaerythritol triacrylate, tris (2-hydroxy ethyl) isocyanurate triacrylate, isocyanuric acid tris(2-acryloyloxyethyl) ester, ethoxylated trimethylolpropane triacrylate, pentaerythrityl triacrylate and glycerol triacrylate, phosphinylidynetris(oxyethylene) triacrylate.

Hydrogels may take a physical form that ranges from soft, brittle and weak to hard, elastic and tough material. Soft hydrogels may be characterized by rheological parameters including elastic and viscoelastic parameters, while hard hydrogels are suitably characterized by tensile strength parameters, elastic, storage and loss moduli, as these terms are known in the art.

The softness/hardness of a hydrogel is governed inter alia by the chemical composition of the polymer chains, the “degree of cross-linking” (number of interconnected links between the chains), the aqueous media content and composition, and temperature.

A hydrogel, according to some embodiments of the present invention, may contain macromolecular polymeric and/or fibrous elements which are not chemically connected to the main crosslinked network but are rather mechanically intertwined therewith and/or immersed therein. Such macromolecular fibrous elements can be woven (as in, for example, a mesh structure), or non-woven, and can, in some embodiments, serve as reinforcing materials of the hydrogel's fibrous network. Non-limiting examples of such macromolecules include polycaprolactone, gelatin, gelatin methacrylate, alginate, alginate methacrylate, chitosan, chitosan methacrylate, glycol chitosan, glycol chitosan methacrylate, hyaluronic acid (HA), HA methacrylate, and other non-crosslinked natural or synthetic polymeric chains and the likes. Alternatively, or in addition, such macromolecules are chemically connected to the main crosslinked network of the hydrogel, for example, by acting as a cross-linking agent, or by otherwise forming a part of the three-dimensional network of the hydrogel.

In some embodiments, the hydrogel is porous and in some embodiments, at least a portion of the pores in the hydrogel are nanopores, having an average volume at the nanoscale range. In some of any of the embodiments described herein, a curable material, whether monomeric or oligomeric, can be a mono-functional curable material or a multi-functional curable material.

Herein, a mono-functional curable material comprises one functional group that can undergo polymerization when exposed to a curing condition (e.g., radiation, presence of calcium ions).

A multi-functional curable material comprises two or more, e.g., 2, 3, 4 or more, functional groups that can undergo polymerization when exposed to curing energy. Multi-functional curable materials can be, for example, di-functional, tri-functional or tetra-functional curable materials, which comprise 2, 3 or 4 groups that can undergo polymerization, respectively. The two or more functional groups in a multi-functional curable material are typically linked to one another by a linking moiety, as defined herein. When the linking moiety is an oligomeric moiety, the multi-functional group is an oligomeric multi-functional curable material.

In some embodiments, curable materials are printed as a scaffold (optionally a sacrificial scaffold, as in a support material) and a cellular formulation (cell-containing formulation) is printed in and/or on the scaffold. In some embodiments, one or more formulations in the building formulation comprises a mixture of cellular formulation(s) (e.g., a mixture of cells) and (e.g., acellular) curable materials, and the curable (e.g., acellular) materials can be a support material or a model material.

In some embodiments, the tumor model (the object) is made of both cellular and curable (e.g., acellular, synthetic, exogenous) modeling materials, and is formed by forming layers of a building material that comprises a plurality of modeling formulations which comprise cellular components (e.g., tumor cells and additional components from its microenvironment, as defined herein) and curable (acellular; synthetic) materials, optionally in combination with acellular (synthetic) curable support material formulations.

The selection of acellular/synthetic (e.g., curable) materials that will compose the bio-ink, in addition to cell suspensions and/or cellular components, in the design of 3D constructs for tissue engineering applications should be made while considering parameters such as biocompatibility, biodegradability, and cell-substrate interactions.

The bio-inks must flow through the deposition nozzle without clogging, yet should solidify (harden) quickly (e.g., within a time period of no more than a few minutes). Hence, the ink is preferably both shear thinning and viscoelastic, i.e., with a shear elastic modulus (G′) that exceeds the loss modulus (G″).

According to some embodiments of the present invention, the bio-ink (e.g., the one or more modeling material formulation(s)) comprises cellular components, as described herein, and may further comprise curable (e.g., acellular, exogenous/synthetic) components, as described herein.

In some embodiments, the curable material(s) are selected so as to provide the tumor model with chemical, mechanical and/or physical properties that correspond to the respective properties of the tumor, as explained hereinafter.

Curable materials usable in the field of bioprinting are predominantly based on either naturally derived materials (including, for example, Matrigel, Alginate, Pectin, Xanthan gum, Gelatin, Collagen, Chitosan, Fibrin, Cellulose and Hyaluronic acid, often isolated from animal or human tissues) or synthetically-prepared materials (including, for example, polyethyleneglycol; PEG, gelatin methacrylate; GelMA, poly(propylene oxide); PPO, poly(ethylene oxide); PEO), all of which are referred to herein as curable materials that form a synthetic material. Naturally derived materials for 3D bioprinting are advantageous due their similarity to human ECM, and their inherent bioactivity. Synthetically-prepared materials are advantageous in that they can be tailored with specific physical and/or mechanical properties to suit particular applications.

In some embodiments, a curable material, whether it is naturally derived or synthetically-prepared, is a material that forms, upon curing, a synthetic material as described herein (e.g., a material exogenous to the tumor and its environment and/or the subject).

Synthetic materials and/or curable materials forming synthetic materials as described herein can be degradable or non-degradable materials, and may include, for example, hydrogels made of one or more polymers (PEG, polyethyleneglycol-diacrylate, polyglutamic acid, gelatin methacrylate; GelMA, poly(propylene oxide); PPO, poly(ethylene oxide); PEO, PLGA/PLLA), poly(dimethyl siloxane); Nanocellulose; Pluronic F127, or short di-peptides (FF) and Fmoc-peptide-based hydrogel (Fmoc-FF-OH, Fmoc-FRGD-OH, Fmoc-RGDF-OH, Fmoc-2-Nal-OH, Fmoc-FG-OH). Thermoplastic polymers such as Polycaprolactone (PCL), Polylactic acid (PLA) or Poly(D,L-lactide-co-glycolide) along with silicone inks can be used to create customized templates and molds.

The following describes exemplary curable materials usable in the context of the present embodiments:

Gelatin is a low-cost, abundant and biocompatible material, composed of hydrolyzed collagen. The amino acid content of hydrolyzed collagen is the same as collagen.

The term is meant to encompass also analogs of gelatin such as Gelatin methacrylate (GelMA). GelMa is a low-cost, abundant and biocompatible material, composed of denatured collagen that is modified so as to undergo cross-linking when exposed irradiation, preferably in the presence of a photoinitiator. Gelatin is modified with photopolymerizable methacrylate (MA) groups, resulting in a matrix that can be cross-linked through free radical polymerization by short exposure to UV light after printing. By modulating the concentration, degree of methacrylation, and temperature, the shear yield stress and clastic modulus of cured GelMA-containing formulations can be tuned.

Pluronic® materials are class of triblock co-polymers based on Poly-ethylene oxide and Poly-propylene oxide which exhibit reverse thermal gelation. For example, Pluronic F127 is fluid at a low temperature forms a gel at a high temperature, above critical micellar concentration (CMC). Pluronic F127 can be used either as a sacrificial (support) material or be mixed with cellular components. Pluronic F127-diacrylate (DA) is also UV-curable and can be used as an integral part of the final structure.

Fibrin is a glycoprotein in vertebrates that has an important role in the formation of blood clots. Fibrinogen can form a gel when mixed with thrombin, to form fibrin gel. However, fibrin suffers from two main limitations: (i) it has quite poor mechanical properties, and (ii) its gelation process can be too fast from a printing prospective. Therefore, there is a need for a special core-sheath nozzle or post-crosslinking process to avoid gelation prior to extrusion combined with a thickener agent such as pure gelatin which can afterwards be crosslinked to the fibrin gel with Transglutaminase (TG) or some anionic polysaccharide such as Alginate, Xanthan gum or Pectin which can afterwards be crosslinked when the gel is inserted to a cell-media. Combination of PLLA/PLGA sponges with fibrin matrices provides additional mechanical strength (23).

Clay mineral and carbon nanotubes can be included in each of the materials mentioned above to improve the mechanical properties of soft hydrogels and grant electrical properties which can be beneficial to modeling of brain tumors.

According to some embodiments of the present invention, the curable materials in the building material formulation (bio-ink) were selected so as to provide the tumor model with chemical, mechanical and/or physical properties that match the original tumor, as described herein.

For example, for tumors residing in soft tissues such as brain, curable materials that provide synthetic hardened materials exhibiting Young's modulus of about 1-30 kPa are used which is similar to that of GB or metastatic brain tumor.

In exemplary embodiments of the present invention, the bio-ink formulation comprises an enzymatic system, such that the curing is effected by means of one or more enzymatically-catalyzed reactions. The use of such formulations allow controlling the properties of the hardened material by controlling the enzymatically catalyzed reactions, for example, by selecting suitable concentrations of the enzymes.

In exemplary embodiments of the present invention, the tumor bio-ink formulation comprises fibrinogen 0.5-4%, 0.5-3.5%, 0.5-3%, 0.5-2.5%, 0.5-2%, 0.5-1.5%, 1-4%, 1-3%, weight/volume (w/v).

In exemplary embodiments of the present invention, the tumor bio-ink formulation comprises gelatin 1-20% w/v, e.g., 1-18% w/v, 1-16% w/v, 1-14% w/v, 1-12% w/v, 1-10% w/v, 1-8% w/v, 2-20% w/v, 4-20% w/v, 6-20% w/v, 8-20% w/v, 10-20% w/v, 4-8% w/v, e.g., 6% w/v.

In exemplary embodiments of the present invention, the tumor bio-ink formulation comprises

According to a specific embodiment, a concentration of said thrombin, if present, in said curable formulation, ranges from 0.1 to 5, or from 0.1 to 4, or from 0.1 to 3, or from 0.1 to 2, or from 0.1 to 1, or is 0.5, U/ml.

According to a specific embodiment, a concentration of said transglutaminase, if present, in said curable formulation, ranges from 0.1 to 50, 0.1 to 10, or from 1 to 10, or from 1 to 5, or is 3, % by volume of the total volume of the formulation.

According to a specific embodiment, said first synthetic material comprises fibrin, gelatin, thrombin and transglutaminase (TG) and CaCl₂.

According to a specific embodiment, the tumor bio-ink formulation comprises fibrinogen 0.5-4%, 0.5-4%, 0.5-4%, 0.5-4%, 0.5-4%, 0.5-4%, 0.5-4%, 0.5-4%, 0.5-4%, weight/volume (w/v), e.g., 1% w/v, gelatin 1-20% w/v, e.g., 6%, thrombin 0.05-2 U/ml, e.g., 0.5 U/ml and TG 1-50% w/v. e.g., 3% w/v, the latter used for promoting cross linking of the fibrin by the anionic polymer, i.c., the gelatin.

Optionally, additional acellular agents, curable or non-curable are added to one or more formulations, to further alter the mechanical properties of the hardened material.

In some of any of the embodiments described herein, the bio-ink formulation comprises, in addition to the curable material, a suitable medium for maintaining viability and/or proliferation of the cells in the tumor model.

According to some embodiments, the tumor bio-ink is composed of fibrin, gelatin and thrombin, cross-linked with transglutaminase (TG).

Fibrinogen is combined with the hydrolyzed form of collagen, gelatin, a low-cost, abundant and biocompatible material, and the major component of the ECM in most tissues. Slow crosslinking of the fibrin gel is allowed by transglutaminase (TG), a natural, non-toxic enzyme. TG can catalyze intra- and inter-molecular covalent bonds between glutamine and lysine residues of gelatin.

According to some embodiments, gelatin is prepared by dissolving in phosphate-buffered saline (PBS) without calcium and magnesium under appropriate conditions selected from temperature, e.g., at 70° C. for a few hours e.g., between 2-24 hours e.g., 12 h under stirring. Then, according to some embodiments, the pH is adjusted dropwise to 7.5 such as by using 1 M NaOH. The solution can be filtered through 0.2 μm filter and stored at 4° C. Fibrinogen solution is produced by dissolving lyophilized human blood plasma protein at 37° C. in sterile PBS without calcium and magnesium for a time period sufficient for dissolving, e.g., 45 min. The pH is adjusted dropwise to 7.5 using 0.5 M NaOH and the solution can be stored at −20° C. Transglutaminase (TG) solution (100 mg/ml) is dissolved in PBS without calcium and magnesium, gently mixed for time and temperature (e.g., 20 min at 37° C.) sufficient to obtain a well stirred composition and sterile-filtered. Stock solution of 250 mM CaCl₂ is prepared by dissolving CaCl₂ powder in PBS without calcium and magnesium. To prepare stock solution of thrombin, lyophilized thrombin is reconstituted such as at 2000 U/ml using sterile PBS without calcium and magnesium and stored at −20° C.

According to a specific embodiment, CaC12 is present in the tumor bio-ink e.g., 0.01-10 mM. 0.5-10 mM, 1-5 mM, e.g., 2.5 mM.

According to a specific embodiment, a second synthetic material is used to create the vascular bio-ink.

According to some embodiments, the vascular bio-ink is composed of Pluronic F-127 36-38% w/v and optionally thrombin.

It will be appreciated that once fabricated, the 3D model is essentially devoid of the synthetic material (i.e., second synthetic material) from which the vasculature is produced because it is liquified/melted and depleted during the production process.

As used herein “essentially” refers to less than 21% w/v of Pluronic in the 3D model (e.g., less than 15%, 10%, 5%, 1%, 0.1%, 0.01% w/v).

### Microengineered Blood Vessels:

A 3D tumor model as described herein is populated with living cells.

In some embodiments, the method further comprises perfusing the 3D-bioprinted model of the tumor, for example, by creating blood vessels during the bioprinting process, as described herein.

In some embodiments, the tumor model further comprises small diameter blood vessels.

According to a specific embodiment, the vasculature is coated by the tumor/stroma bio-ink and/or is not in direct contact with the receiving medium (glass).

Achieving vascularization of the desired 3D tumor model, for example, in order to test different drugs on it, is considered a major challenge in bioprinting. Several 3D printers are capable building tiny, hierarchical networks of blood vessels to supply blood.

One approach for achieving vascularization comprises using a customized, high-resolution 3D printer that can form microchannels in biocompatible gels. These hydrogel materials can be printed at the micron-length scale (the smallest microvascular channels that are printed are around 10 microns in diameter). Using this approach, a capillary network of fluorescently labeled sacrificial ink is printed into gel-like matrix which can be melted later. Further, blood vessels can be printed using sacrificial template of carbohydrate glass and seeded with endothelial cells (ECs), such that the ECs line the interiors of the channels and may penetrate the surrounding cell-gel mixture).

In some embodiments, the 3D tumor model described herein comprises a functional perfusable vascular system with active flow, which mimics high pressure pulsatile blood flow, hemodynamics, shear stress, etc. Vascularization is important as it maintains cell viability and encourages tissue organization and differentiation.

In some embodiments of the present invention, a 3D tumor model featuring interconnected channels is manufactured as described herein, and a Pluronic solution, optionally containing, e.g., cells, factors and/or any other biological materials present in the microenvironment of the tumor, is creating the channels, forming a network of microchannels that mimics a vascularized tumor.

An exemplary methodology, which utilizes Pluronic for forming a vascular network in the tumor model is demonstrated in the Examples section that follows.

Thus, a vascular bio-ink is composed of 21-40% w/v Pluronic F-127, 30-40% w/v Pluronic F-127, 35-40% w/v Pluronic F-127 e.g., 38% w/v and optionally thrombin 0-1000 U/ml, 5-1000 U/ml, 10-1000 U/ml, 20-1000 U/ml, 30-1000 U/ml, 40-1000 U/ml, 50-1000 U/ml, 60-1000 U/ml, 70-1000 U/ml, 80-1000 U/ml, 90-1000 U/ml, 100-1000 U/ml, 50-1000 U/ml, 50-500 U/ml, 50-400 U/ml, 50-300 U/ml, 50-200 U/ml, e.g., 100 U/ml.

The 3D printed model is arranged by printing tumor and stroma cells and vasculature in a layered manner.

An exemplary embodiment of the printing method is provided infra.

Thus, a printing formulation of fibrin 3D-bio-ink (composed of 1% w/v fibrinogen and 6% w/v gelatin) is mixed with tumor and stroma cells. The mixture is loaded in a syringe and tapered with a needle tip. The bio-ink is cooled to 4° C. and then loaded into the 3D printer's cartridge. Temperature is raised such as to room temperature prior to 3D-bioprinting initiation. 3D structures are printed using a selected system, e.g., 3D-Bioplotter® (Manufacturer series, EnvisionTEC®, Gladbeck, Germany) equipped with a number of independent ink cartridges. Before each 3D-bioprinting, the nozzles are calibrated to determine their respective X, Y and Z offsets. Following calibration, a number of layers are printed e.g., 6 to 8 layers on a thin coverslip, typically framed such as by a polydimethylsiloxane (PDMS) (Sylgard™ 184, Dow®, Michigan, USA) gasket, creating the bottom platform onto which vasculature structure can be printed. Cell-laden bio-inks are printed for a time period such as up to 2 h to prevent cell death. The printed layers are left to dry. Then, the vascular bio-ink composed of Pluronic F127 38% w/v and thrombin 100 U/ml is loaded onto a printer cartridge at 4° C., warmed to 29° C., and printed using a (e.g., 0.25 mm) needle tip attached via a luer-lock. 3D structures are printed with the vascular bio-ink according to a bioengineered design of vasculature, such as created with Rhino 6® 3D modeling software (Rhinoceros®). Immediately after the vascular bio-ink 3D-bioprinting is completed, connectors (e.g., such as available from Darwin microfluidics, Paris, France) are inserted to the inlet and outlet positions of the PDMS gasket to allow future flow by peristaltic pump, and a fresh formulation of the tumor bio-ink with cancer and microenvironment cells is casted, covering the printed vasculature and filling the frame completely. The sample is then covered with additional coverslip glass and sealed in a metal frame (creating the perfusable chip), and incubated at 37° C. until complete crosslinking is achieved, reaching the physiological stiffness of the brain.

To liquefy the fugitive Pluronic F127 bio-ink, the sample is cooled to 4° C. Cold buffer, e.g., PBS is then injected into the mold's inlet by applying positive pressure, while applying negative pressure through the outlet leaving the model with the desired 3D-bioprinted lumens. Following Pluronic F127 wash, fibronectin (e.g., 100 μg/ml) or any other adherent biomaterial e.g., laminin, collagen, PLL is injected into the lumens' inlet and incubated in rotation at 37° C. to prime the vasculature wall and create an adherable interface. Next, a mixture of endothelial cells and pericytes (named vascular bio-ink) is injected into the vessel and incubated.

According to some embodiments, the cell seeding step is repeated 2-6 e.g., 4 times.

According to some embodiments, each time sample incubation positioning is rotated by 90 degrees to maximize cell attachment area. The model is then incubated in rotation at 37° C. to allow cell attachment to the lumens' walls with a full coverage of the lumen.

According to some embodiments, the model is connected to a peristaltic pump (e.g., EBERS, Zaragoza, Spain), incubated at 37° C. and perfused with medium mixture of all the cells in the samples e.g., at a 1:1:1:1:1 ratio (DMEM, astrocyte medium, microglia medium, pericyte medium and EGM-2) for a number of days, e.g., 4-10 days. Confluence can be validated by imaging.

### A Tumor Model:

According to some embodiments of the present invention the three dimensional (3D) model of a tumor comprises a plurality of cell types having a full HLA match, the plurality of cell types comprising malignant cells and non-malignant cells (stoma) of the tumor as well as vasculature. The 3D model tumor is also referred to herein as a bioprinted tumor model or as a 3D bioprinted tumor model.

According to some of these embodiments, the plurality of cell types are arranged in high matchability to a 3D image (e.g., obtained by a 3D imaging technique as described herein) of the tumor. The matchability of the 3D arrangement of the cell types to the 3D image is, and can be determined, as described herein above.

According to some embodiments, a ratio of said endothelial cells and said pericytes in the vasculature or seeding solution ranges from 2:1 to 10:1, e.g., 4:1.

According to some embodiments, a ratio of said endothelial cells and said pericytes in the vasculature or seeding solution ranges from 3:1 to 10:1.

According to some embodiments, a ratio of said endothelial cells and said pericytes in the vasculature or seeding solution ranges from 4:1 to 10:1.

According to some embodiments, a ratio of said endothelial cells and said pericytes in the vasculature or seeding solution ranges from 5:1 to 10:1.

According to some embodiments, a ratio of said endothelial cells and said pericytes in the vasculature or seeding solution ranges from 6:1 to 10:1.

According to some embodiments, a ratio of said endothelial cells and said pericytes in the vasculature or seeding solution ranges from 7:1 to 10:1.

According to some embodiments, a ratio of said endothelial cells and said pericytes in the vasculature or seeding solution ranges from 8:1 to 10:1.

According to some embodiments, a ratio of said endothelial cells and said pericytes in the vasculature or seeding solution ranges from 9:1 to 10:1.

According to some embodiments, a ratio of said endothelial cells and said pericytes in the vasculature or seeding solution ranges from 2:1 to 9:1.

According to some embodiments, a ratio of said endothelial cells and said pericytes in the vasculature or seeding solution ranges from 2:1 to 8:1.

According to some embodiments, a ratio of said endothelial cells and said pericytes in the vasculature or seeding solution ranges from 2:1 to 7:1.

According to some embodiments, a ratio of said endothelial cells and said pericytes in the vasculature or seeding solution ranges from 2:1 to 6:1.

According to some embodiments, a ratio of said endothelial cells and said pericytes in the vasculature or seeding solution ranges from 2:1 to 5:1.

According to some embodiments, a ratio of said endothelial cells and said pericytes in the vasculature or seeding solution ranges from 2:1 to 6:1.

According to some embodiments, a ratio of said endothelial cells and said pericytes in the vasculature or seeding solution ranges from 2:1 to 5:1.

According to some embodiments, the vasculature is configured to provide perfusion at a range of shear stress of 0.01-100 dyn/cm² or 5-1000 μl/min, for a vessel of 1 mm in diameter of the vasculature.

According to some embodiments, the vasculature is configured to provide perfusion at a range of shear stress of 2-20 dyn/cm², for a vessel of 1 mm in diameter of the vasculature.

According to some embodiments, the vasculature is configured to provide perfusion at shear stress of 25 μl/min for a vessel 1 mm in diameter of said vasculature.

According to some embodiments, a density of the tumor cells in the model is between 0.1×10⁵-1×10⁸ cells/ml (or multiplied by 1000 to reach cells/mm3 units).

According to some embodiments, a density of the tumor cells is about 1×10⁶-1×10⁷ cells/ml.

According to some embodiments, a ratio of said tumor cells to said astrocytes and microglia cells is in the range of 20:1 to 1:10. Astrocytes: 1×10⁵-1×10⁶ cells/ml, Microglia: 1×10³-1×10⁶ cells/ml.

According to some embodiments, a ratio of said tumor cells to said astrocytes and microglia cells is in the range of 18:1 to 1:10.

According to some embodiments, a ratio of said tumor cells to said astrocytes and microglia cells is in the range of 15:1 to 1:10.

According to some embodiments, a ratio of said tumor cells to said astrocytes and microglia cells is in the range of 12:1 to 1:10.

According to some embodiments, a ratio of said tumor cells to said astrocytes and microglia cells is in the range of 10:1 to 1:10.

According to some embodiments, a ratio of said tumor cells to said astrocytes and microglia cells is in the range of 8:1 to 1:10.

According to some embodiments, a ratio of said tumor cells to said astrocytes and microglia cells is in the range of 6:1 to 1:10.

According to some embodiments, a ratio of said tumor cells to said astrocytes and microglia cells is in the range of 4:1 to 1:10.

According to some embodiments, a ratio of said tumor cells to said astrocytes and microglia cells is in the range of 2:1 to 1:10.

According to some embodiments, a ratio of said tumor cells to said astrocytes and microglia cells is in the range of 1:1 to 1:10.

According to some embodiments, a ratio of said tumor cells to said astrocytes and microglia cells is in the range of 20:1 to 9:10.

According to some embodiments, a ratio of said tumor cells to said astrocytes and microglia cells is in the range of 20:1 to 8:10.

According to some embodiments, a ratio of said tumor cells to said astrocytes and microglia cells is in the range of 20:1 to 7:10.

According to some embodiments, a ratio of said tumor cells to said astrocytes and microglia cells is in the range of 20:1 to 6:10.

According to some embodiments, a ratio of said tumor cells to said astrocytes and microglia cells is in the range of 20:1 to 5:10.

According to some embodiments, a ratio of said tumor cells to said astrocytes and microglia cells is in the range of 20:1 to 4:10.

According to some embodiments, a ratio of said tumor cells to said astrocytes and microglia cells is in the range of 20:1 to 3:10.

According to some embodiments, a ratio of said tumor cells to said astrocytes and microglia cells is in the range of 20:1 to 2:10.

According to some embodiments, a ratio of said tumor cells to said astrocytes and microglia cells is in the range of 20:1 to 1:10.

According to some embodiments, a ratio of said tumor cells to said astrocytes and microglia cells is in the range of 1:10 to 10:1.

According to some embodiments, a ratio of said tumor cells to said astrocytes and microglia cells is in the range of 1:8 to 10:1.

According to some embodiments, a ratio of said tumor cells to said astrocytes and microglia cells is in the range of 1:6 to 10:1.

According to some embodiments, a ratio of said tumor cells to said astrocytes and microglia cells is in the range of 1:2 to 10:1.

According to some embodiments, a ratio of said tumor cells to said astrocytes and microglia cells is in the range of 1:1 to 10:1.

According to some embodiments, a ratio of said tumor cells to said astrocytes and microglia cells is in the range of 1:10 to 8:1.

According to some embodiments, a ratio of said tumor cells to said astrocytes and microglia cells is in the range of 1:10 to 6:1.

According to some embodiments, a ratio of said tumor cells to said astrocytes and microglia cells is in the range of 1:10 to 2:1.

According to some embodiments, a ratio of said tumor cells to said astrocytes and microglia cells is in the range of 1:10 to 1:1.

According to some embodiments, a ratio of said tumor cells to said astrocytes and microglia cells is in the range of 1:1 to 5:1.

According to some embodiments, a ratio of said tumor cells to said astrocytes and microglia cells is in the range of 1:1 to 2:1 or 1:2.

According to some embodiments, a ratio of said tumor cells to said astrocytes and microglia cells is in the range of 1:1 to 4:1. According to some embodiments, a ratio of said tumor cells to said astrocytes and microglia cells is in the range of 1:1 to 3:1.

According to some embodiments, a ratio of said tumor cells to said astrocytes and microglia cells is in the range of 1:1 to 2:1.

According to some embodiments, the model is characterized by at least one of:
(i) porosity of 1-40 μm (e.g., 5-40, 10-40, 20-40, 1-30, 1-20, 1-10 μm);. These numbers typically convey an increase of at least 20%, 50%, 100%, 200%, 300%, 500% as compared to pores found in healthy brain tissue of the environment of the tumor.
(ii) cell viability for at least 4 weeks in physiological conditions (37° C., 5% CO₂);
(iii) stiffness of 5-25 kPa Young's Modulus;
(iv) swelling equilibrium of 5-25%;
(v) perfusability for at least 1 day, e.g., at least 5 days, 8 days, 10 days, 14 days; and/or
(vi) activation of said microglia and/or astrocytes.

As described in the Examples section which follows, the present inventors evaluated the structure of the model using scanning electron microscopy (SEM) in comparison to healthy and tumor bearing mice brain tissues from C57BL/6 mice. Acellular fibrin 3D-bio-ink and healthy hemisphere show smaller pores compared to the larger pores observed in the cell-laden fibrin 3D- tumor bio-ink and tumor containing hemisphere. Nonhomogeneous structure and an average pore size of 4.7 μm in diameter was measured in acellular fibrin 3D-bio-ink; therefore, small molecules such as oxygen, nutrients and drug molecules can diffuse freely through the model. The pore size of the tumor-bio-ink is significantly increased when tumor cells are seeded alone or co-cultured with brain stromal cells in the 3D-bio-ink (7.2-7.1 μm diameter, respectively). The addition of activated and proliferating tumor and stromal cells alters the appearance of synthetic and natural ECM and significantly increase their pore size (p=2×10^{-13,} t-test). Similarly, the pore size of the tumor containing hemisphere is higher than the pore size in a healthy hemisphere (p=0.0002, t-test). Additionally, the cell-laden fibrin 3D-bio-ink show cellular filopodia towards the surrounding matrix, suggesting the presence of cell-cell and cell-ECM interactions. As used herein “exhibits cell viability” means maintains viability of the cells at day 0 (immediately before printing)±about 30%, 20%, 15%, 10%, 5% following printing as determined in a predetermined viability assay.

Viability or proliferation are measured by PrestoBlue, AlamarBlue or Trypan Blue assay.

In some embodiments, the plurality of cell types exhibits viability for at least 30 days following printing.

In some embodiments, the plurality of cell types exhibits viability for at least 20 days following printing.

In some embodiments, the plurality of cell types exhibits viability for at least 15 days following printing.

In some embodiments, the plurality of cell types exhibits viability for at least 14 days following printing.

In some embodiments, the plurality of cell types exhibits viability for at least 13 days following printing.

In some embodiments, the plurality of cell types exhibits viability for at least 12 days following printing.

In some embodiments, the plurality of cell types exhibits viability for at least 11 days following printing.

In some embodiments, the plurality of cell types exhibits viability for at least 10 days following printing.

In some embodiments, the plurality of cell types exhibits viability for at least 9 days following printing.

In some embodiments, the plurality of cell types exhibits viability for at least 7 days following printing.

In some embodiments, the plurality of cell types exhibits a proliferative capacity for at least 30 days following printing.

As used herein “exhibits a proliferative capacity” means maintains proliferation potential (e.g., doubling rate) of the cells at day 0 (immediately before printing) +about 30%, 20%, 15%, 10%, 5% following printing as determined in a predetermined proliferation assay.

In some embodiments, the plurality of cell types exhibits a proliferative capacity for at least 20 days following printing.

In some embodiments, the plurality of cell types exhibits a proliferative capacity for at least 15 days following printing.

In some embodiments, the plurality of cell types exhibits a proliferative capacity for at least 14 days following printing.

In some embodiments, the plurality of cell types exhibits a proliferative capacity for at least 13 days following printing.

In some embodiments, the plurality of cell types exhibits a proliferative capacity for at least 11 days following printing.

In some embodiments, the plurality of cell types exhibits a proliferative capacity for at least 10 days following printing.

In some embodiments, the plurality of cell types exhibits a proliferative capacity for at least 9 days following printing.

In some embodiments, the plurality of cell types exhibits a proliferative capacity for at least 8 days following printing.

In some embodiments, the plurality of cell types exhibits a proliferative capacity for at least 7 days following printing.

In some of any of the embodiments described herein, the 3D model comprises a perfusable vasculature (e.g., as described herein) since they comprise a lumen.

The present inventors were able to perfuse the vasculature with 70 kDa dextran-FITC solution using a syringe pump and monitored the flow of dextran-FITC by fluorescent microscopy.

The use of the Dextran FITC shows the potential to perfuse the sample through the vessels that are used as channels to transport nutrients and waste into and out of the system, respectively. These perfusable blood vessels offer the possibility to mimic the blood brain barrier allowing the evaluation of the integrity and functionality of the endothelial barrier in the presence or absence of cancer cells. Indeed, in a more clinically relevant setting the present inventors perfused a chemotherapy in the model as shown in the Examples section. The present inventors have also shown that the stroma is active in supporting the growth of the tumor cells suggesting activation of astrocytes and microglial cells in the model. In some of any of the embodiments described herein, there is provided a 3D tumor model obtainable by the bioprinting method as described herein. In some of these embodiments, the tumor is characterized by one or more of the above-described features. A tumor model obtainable by a bioprinting method as described herein is also referred to herein as a 3D-bioprinted tumor model.

In any of the aspects and embodiments as described herein, a “3D tumor model” or a “3D model of a tumor” are used interchangeably and refer to a model tumor as described herein and to a 3D-bioprinted tumor model as described herein.

In some of any of the embodiments described herein, the 3D tumor model and its blood vessels are connected through fluidic conduits which function as a synthetic circulatory system (see FIGS. 3A-B).

Such 3D-microengineered printed tumors open new avenues for drug screening and fundamental studies of the tumor microenvironment as well as for both surgical and research purposes, as described in further detail hereinafter.

### Applications:

The 3D model of a tumor as described herein is usable in various applications, including research (e.g., for drug design, drug screening, simulating surgery) and for the purpose of evaluating an operative anti-cancer regimen suitable for the specific tumor and subject having same.

The 3D models can be produced, stored, distributed, marketed, advertised, and sold as, for example, kits for biological assays and high-throughput drug screening. In other embodiments, the 3D models are produced and utilized to conduct biological assays and/or drug screening as a service.

According to an aspect of some embodiments of the present invention there is provided a method of screening for an anti-cancer treatment regimen, the method comprising: subjecting a 3D model or system as described herein (with or without perfusion as described herein) of a tumor as described herein to the anti-cancer treatment regimen; and determining a presence of an anti-cancer effect (e.g., inhibition of tumor growth, killing of cancer cells, inducing apoptosis of cancer cells, anti-angiogenic effect) of the anti-cancer treatment regimen on the tumor.

This, according to a specific embodiment, subjecting refers to contacting for a predetermined time period.

According to another embodiment, subjecting refers to perfusion such as using the system as described herein.

Perfusion can be of biological samples such as blood or fractions thereof e.g., PBMC, serum, sub-popylations thereof e.g., myeloid cells e.g., macrophages, NK cells, T-cells, which are naturally occurring or engineered such as to express a chimeric T cell receptor (CAR).

Perfusion can be of small molecules or biological molecules such as polypeptides, peptides, antibodies, nucleic acid agents (e.g., RNA silencing agent, DNA/RNA editing agents), carbohydrates, lipids or combinations of same.

According to a specific embodiment, the perfused substance is an FDA-approved drug.

According to a specific embodiment, the perfused substance is a research/test molecule.

According to some embodiments, the perfused substance is chemically defined (with above 95% certainty regarding the identity of the chemical content).

According to some embodiments, the perfused substance is chemically undefined (e.g., blood or fractions thereof or conditioned medium).

The anti-cancer treatment regimen can be any one of a chemotherapy, a radiotherapy, an immunotherapy, a thermotherapy, a hormonal therapy, and any combination thereof.

Specific GB first line or second line treatments (for recurrent tumors) include, but are not limited to, Temozolomide (TMZ), Biodegradable carmustine wafers, biological therapies such as targeted therapies and immune checkpoint modulators, dose-fractionation schedule for external beam RT, antiangiogenic therapeutic strategies such as Bevacizumab an anti circulating VEGF-A, nitrosoureas-DNA alkylating agents, namely carmustine (BCNU), lomustine (CCNU), nimustine (ACNU), and fotemustine, immunotherapy with a focus on immune checkpoint inhibitors, myeloid-targeted therapies, vaccines and chimeric antigen receptor (CAR) immunotherapies (Yu and Quail Front. Immunol., 13 May 2021

Sec. Cancer Immunity and Immunotherapy, which is hereby incorporated by reference in its entirety).

Various treatments for GB are described in Fernandes et al. Chapter 11 Current Standards of Care in Glioblastoma Therapy, which is hereby incorporated by reference in its entirety.

Treatments for metastatic brain tumors include, but are not limited to:

Targeted therapies such as but not limited to trastuzumab for breast cancer that has spread to the brain and Erlotinib for the most common type of lung cancer (non-small cell lung cancer) that has spread to the brain.

Immunotherapy drugs to treat metastatic brain tumors include, but are not limited to, Atezolizumab, Ipilimumab, Pembrolizumab, Nivolumab.

Radiation therapy, e.g., external beam therapoy, whole-brain radiation, proton therapy, and brachytherapy.

Various assays can be used to determine the effect of the anti cancer agent/regimen. Some non-limiting examples are described herein below.

According to a specific embodiment, the “assay” is a procedure for testing or measuring the presence or activity of a substance (e.g., a chemical, molecule, biochemical, drug, physical condition e.g., radiation, etc.) in the 3D model.

In further embodiments, assays include qualitative assays and quantitative assays. In still further embodiments, a quantitative assay measures the amount of a substance in a sample.

In various embodiments, the assay is selected from the group consisting of an image-based assays, measurement of secreted proteins, expression of markers, and production of proteins.

In various further embodiments, the 3D models as describe herein are for use in assays to detect or measure one or more of: molecular binding (including radioligand binding), molecular uptake, activity (e.g., enzymatic activity and receptor activity, etc.), gene expression, protein expression, receptor agonism, receptor antagonism, cell signaling, apoptosis, chemosensitivity, transfection, cell migration, chemotaxis, cell viability, cell proliferation, safety, efficacy, metabolism, toxicity, and abuse liability.

In various further embodiments, the 3D models as describe herein are for use in immunoassays. In further embodiments, immunoassays are competitive immunoassays or noncompetitive immunoassays. In a competitive immunoassay, for example, the antigen in a sample competes with labeled antigen to bind with antibodies and the amount of labeled antigen bound to the antibody site is then measured. In a noncompetitive immunoassay (also referred to as a “sandwich assay”), for example, antigen in a sample is bound to an antibody site; subsequently, labeled antibody is bound to the antigen and the amount of labeled antibody on the site is then measured.

According to a specific embodiment, the immunoassay assays the effect of immune cells (e.g., autologous or non-autologous e.g., allogeneic) on the tumor. Such cells can be obtained from the blood e.g., PBMC and tested in the above described system.

Immune cells can include, but are not limited to, the innate immune cells, adaptive immune cells or components thereof.

The immune cells can be provided in a biological sample (e.g., serum) or alternatively in a culture medium.

It will be appreciated that the effect of various factors in a medium can be tested also in the absence of immune cells.

The terms “medium”, “cell culture medium”, “culture medium”, and “growth medium” as used herein refer to a solution containing nutrients which nourish growing eukaryotic cells. Typically, these solutions provide essential and non-essential amino acids, vitamins, energy sources, lipids, and trace elements required by the cell for minimal growth and/or survival. The solution can also contain components that enhance growth and/or survival above the minimal rate, including hormones and growth factors. The solution is formulated to a pH and salt concentration optimal for cell survival and proliferation. The medium can also be a “defined medium” or “chemically defined medium”—a serum-free medium that contains no proteins, hydrolysates or components of unknown composition. Defined media are free of animal-derived components and all components have a known chemical structure. One of skill in the art understands a defined medium can comprise recombinant polypeptides or proteins, for example, but not limited to, hormones, cytokines, interleukins and other signaling molecules.

In various further embodiments, the 3D models as describe herein are for use in drug screening or drug discovery. In further embodiments the 3D model is used as part of a kit for drug screening or drug discovery. In some embodiments, each 3D model exists within a well of a biocompatible multi-well container, wherein the container is compatible with one or more automated drug screening procedures and/or devices. In further embodiments, automated drug screening procedures and/or devices include any suitable procedure or device that is computer or robot-assisted.

In various further embodiments, the 3D models as describe herein are for use in research or develop drugs potentially useful in any therapeutic area including anti-cancer efficacy, pharmacology, toxicology, and immunology.

In a particular embodiment, the 3D model as describe herein is for use to identify therapies potentially useful in the disease or condition of a particular individual. In further embodiments, the methods include applying a candidate therapeutic agent or condition to the 3D model; measuring viability of the cells; and selecting a therapeutic agent for the individual based on the measured viability of the cells. In still further embodiments, the candidate therapeutic agent is a one or more chemotherapeutic compounds, one or more radiopharmaceutical compounds, radiation therapy, immune modulator (e.g., checkpoint modulator) or a combination thereof. Accordingly, disclosed herein are methods of personalizing medicine to a subject in need thereof.

According to an aspect of some embodiments of the present invention there is provided a method of characterizing a tumor, the method comprising:

providing the 3D model of the tumor as described herein (e.g., using a bioprinting method as described herein); isolating cells of the tumor model; and in vitro or in vivo culturing the cells. The cultured cells can thereafter be subjected to a variety of methodologies for characterizing the tumor as well as other examples referring to cell proliferation, viability, gene expression etc.

In some embodiments, characterizing the tumor comprises subjecting the cells to an anti-cancer treatment during the culturing, as described herein.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof. Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Materials and Methods

### Materials

Dulbecco's modified eagle medium (DMEM) (Gibco), fetal bovine serum (FBS) (Gibco), L-glutamine, penicillin, streptomycin, mycoplasma detection kit, EZ-RNA II total RNA isolation kit and fibronectin (1 mg/ml, dilution: 1:100) were purchased from Biological Industries Ltd. (Kibbutz Beit HaEmek, Israel). Percoll medium (Cat. No. p4937), Gelatin (Type A, 300 bloom from porcine skin), Pluronic F127, 70 kDa dextran-FITC and all other chemical reagents, including salts and solvents, were purchased from Sigma-Aldrich (Rehovot, Israel). Milli-Q water was prepared using a Millipore water purification system. Fibrinogen, Thrombin, Poly-L-Lysine (PLL) (Cat. No. A-005-C; 0.1 mg/ml), Endo-Gro medium and Bovine Serum Albumin were purchased from Merck Millipore (Burlington, Massachusetts, USA). Transglutaminase was purchased from Moo Glue (Modernist Pantry, Eliot Maine, USA). Collagenase II, Collagenase IV, Dispase II (neutral protease) and DNase I were purchased from Worthington Biochemical Corporation (New Jersey, USA). RBC lysis solution (Cat. No. 420301) was purchased from BioLegend (San Diego, California, USA). MACS MS magnetic columns for cell separation (Cat. No. 130-042-201), CD11b MicroBeads for cell isolation (Cat. No. 130-093-634), CD31 MicroBeads for cell isolation (Cat. No. 130-097-418), anti-AN2 MicroBeads for cell isolation (Cat. No. 130-097-170) and CD144 MicroBeads for cell isolation (Cat. No. 130-097-857) were purchased from Miltenyi Biotec (Bergisch Gladbach, Germany). SELP inhibitor (SELPi) KF38789 (Cat. No. 2748) was purchased from Tocris BioScience (Bristol, United Kingdom). Latex beads for phagocytosis assays (Cat. No. L4655) were purchased from Sigma-Aldrich (Rehovot, Israel). ProLong® Gold mounting with DAPI (Cat. No. p36935) and Hoechst 33342 (Cat. No. H3570) were purchased from Invitrogen (Carlsbad, California, USA). Mayer's Hematoxylin solution (Cat. No. 05-06002) and Eosin Y solution (Cat. No. 05-10002) were purchased from Bio-Optica (Milano, Italy). PresoBlue® Cell Viability Reagent was purchased from ThermoFisher Scientific (Massachusetts, USA). Plasmids: mCherry and iRFP were subcloned into the pQCXIP vector (Clontech, USA) as previously described (25). Primary immunostaining antibodies: Rabbit anti mouse/human GFAP (Dako, Cat. No. 20025480; Lot. No. 41556; 1:500 dilution). Rabbit anti-mouse/human Iba1 (Cat. No. NBP2-19019; Lot. No. 41556; Dilution: 1:200) was purchased from Novus (Colorado, USA). Mouse anti-human SELP (Cat. No. BBA1; Lot. No. APB081704 and APB0818111; Clone BBIG-E; Dilution 1:20) was purchased from R&D Systems (Minneapolis, Minnesota, USA). Secondary immunostaining antibodies: Goat anti-rabbit Alexa Fluor® 488 (Cat. No. ab150077; Lot No. GR315933-2; Dilution 1:300) purchased from Abcam (Cambridge, United Kingdom) and goat anti-mouse Alexa-568 (Cat. No. ab 175473; Lot No. GR3246243-3; Dilution 1:400) was purchased from Jackson ImmunoResearch Laboratories, Inc. (West Grove, Pennsylvania, USA). Flow cytometry antibodies: FITC rat anti-mouse CD62P P-Selectin, clone RB 40.34 (Cat. No. 561923; Lot No. 8340691; Dilution 1:20) and FITC rat IgGI isotype control (Cat. No .; 553995 Lot No.5100888; Dilution 1:20) were purchased from BD Biosciences (New Jersey, USA). Mouse anti-human SELP (Cat. No. BBA1; Lot. No. APB0818111; Clone BBIG-E; Dilution 1:20) and Mouse IgGI isotype control (Cat. No. mab002; Lot. No. IX2417011; Dilution 1:20) were purchased from R&D Systems (Minneapolis, Minnesota, USA). Mousc-IgG k BP-CFL 488 secondary antibody (Cat. No. sc-516176; Lot No. K1119 and HO118; Dilution 1:20) was purchased from Santa Cruz Biotechnology, Inc (Dallas, Texas, USA).

### Fibrin 3D-Bio-Ink

Fibrinogen is a glycoprotein that has an important role in the formation of blood clots in vertebrates; it forms a fibrin gel when mixed with thrombin (Th). Fibrinogen was combined with the hydrolyzed form of collagen, gelatin, a low-cost, abundant and biocompatible material, and the major component of the ECM in most tissues. Slow crosslinking of the fibrin gel was allowed by transglutaminase (TG), a natural, non-toxic enzyme. TG can catalyze intra- and inter-molecular covalent bonds between glutamine and lysine residues of gelatin. Gelatin solutions at 7.5% w/v, 15% w/v and 30% w/v were dissolved in phosphate-buffered saline (PBS) without calcium and magnesium at 70° C. for 12 h under vigorous stirring. Then, the pH was adjusted dropwise to 7.5 using 1 M NaOH. The solution was filtered through 0.2 μm filter and stored at 4° C. Fibrinogen solution (50 mg/ml) was produced by dissolving lyophilized human blood plasma protein at 37° C. in sterile PBS without calcium and magnesium for 45 min. The pH was adjusted dropwise to 7.5 using 0.5 M NaOH and the solution was stored at −20° C. Transglutaminase (TG) solution (100 mg/ml) was dissolved in PBS without calcium and magnesium, gently mixed for 20 min at 37° C. and sterile-filtered. Stock solution of 250 mM CaCl₂ was prepared by dissolving CaCl₂ powder in PBS without calcium and magnesium. To prepare stock solution of thrombin, lyophilized thrombin was reconstituted at 2000 U/ml using sterile PBS without calcium and magnesium and stored at −20° C. The bio-ink solutions were mixed together at 37° C.

### Rheological Characterization

The bio-inks must flow through the deposition nozzle without clogging, yet quickly solidify. To that end, the ink must be both shear thinning and viscoelastic, i.e., with a shear elastic modulus (G′) that exceeds the loss modulus (G″). The ink rheology, mechanical properties and crosslinking kinetics were measured using a controlled stress rheometer (AR-G2, TA Instruments). Time-sweep oscillatory tests in 20 mm cone plate with 1° degree geometry were performed using 350 μl fresh fibrinogen solutions (resulting in a gap size of 600 μm), 1 min after its preparation at 37° C., 0.01% strain and frequency of 1 Hz. The gelation time was determined by the first point at which G′ raises. Each bio-ink formulation was tested three independent times.

### Mechanical Characterization

Young's modulus (E) was evaluated using LS1 tensile testing instrument (Lloyd Instruments Ltd.) equipped with a ultrasensitive 5 N load cell as previously described (26). Young's modulus (E) is defined as:

$$E = \frac{σ}{ε}$$

where σ is the stress applied on the sample and & is the strain in the linear region of the stress-strain curve. Briefly, disk shaped samples of fibrin 3D-bio-ink were prepared as described above, applied into silicone molds (8 mm diameter, 2 mm thickness, Sigma-Aldrich), and incubated at 37° C., 5% CO₂ for 1.5 h. Young's modulus was then evaluated by measuring the slope of the compressive stress-strain curve in the linear region. The samples were compressed at a rate of 1 mm/min up 80% strain. The strain was calculated by determining the initial thickness of the sample at the point at which the stress values changed from negative to positive and started to increase during compressive test. The Young's modulus was calculated from the linear region of the stress-strain curve, as the slope between 1 and 10% strain. Device control, data acquisition, and processing were performed with NEXYGEN plus 3.0 software (Lloyd Instruments Ltd.). Each bio-ink formulation has 6-8 samples per group and the experiments were repeated in three independent times.

### Cell Culture

Human GB cells (U-87MG, T98G and U373), human embryonic kidney 293T (HEK 293T) cells, human osteosarcoma (Saos-2) cells and MDA-MB-231 human breast cancer cells were obtained from the American Type Culture Collection (ATCC, Manassas, Virginia, USA). GL261 murine GB cell line was obtained from the National Cancer Institute (Frederick, Maryland, USA). Cancer cells were grown in DMEM supplemented with 10% fetal bovine serum (FBS) (Gibco), 100 ƒg/ml streptomycin, 100 U/ml penicillin, 12.5 U/ml nystatin and 2 mM L-glutamine. Human astrocytes (hAstro), human microglia (hMG) and primary human microvascular brain pericytes (hPericytes) were purchased from ScienCell (California, USA). hAstro and murine freshly isolated astrocytes (mAstro) cells were grown in the supplied astrocyte medium supplemented with 2% FBS, 100 IU/ml Penicillin, 100 μg/ml Streptomycin, 12.5 U/ml Nystatin, and 1% astrocytes growth supplements (AGS) (ScienCell). Primary hMG and murine freshly isolated microglia (mMG) cells were grown on poly-L-Lysine (PLL, Sigma-Aldrich) coated plates in the supplied microglia medium supplemented with 5% FBS, 100 IU/ml Penicillin, 100 μg/ml Streptomycin, 12.5 U/ml Nystatin, and 1% microglia growth supplements (MGS) (ScienCell). hPericytes were grown on PLL coated plates in the supplied pericytes medium supplemented with 2% FBS, 100 IU/ml Penicillin, 100 μg/ml Streptomycin, 12.5 U/ml Nystatin, and 1% pericytes growth supplements (PGS) (ScienCell). Human umbilical vein endothelial cells (HUVEC) were purchased from Lonza and cultured in EGM-2 medium (Lonza, Switzerland) or Endo-Gro medium on fibronectin coated plates. All cells were grown at 37° C., 5% CO₂. All primary cell lines were grown up to passage 7. Mycoplasma tests were done routinely with a mycoplasma detection kit.

### Ethical Statement

All animals were housed in Tel Aviv University animal facility. The experiments were approved by the animal care and use committee (IACUC) of Tel Aviv University (approval no. approval no. 01-19-015, 01-19-097) and conducted in accordance with NIH guidelines. Experiments involving human tissues were performed with the approval of the Institutional Review Board (IRB) and in compliance with all legal and ethical considerations for human subject research (approval no. 0735-13-TLV). All studies were approved by the local ethics committee and written informed consent was obtained from all patients.

### Human Primary GB Cells

Patient-derived GB (PD-GB) tissues were obtained during surgical procedures from Tel Aviv Sourasky Medical Center (Tel Aviv, Israel) under an approved IRB (0735-12-TLV, 920130237). Tumor tissues were kept in cold PBS and processed within 40 min. Tumor specimens were dissected to 0.5 mm pieces and incubated in rotation with Collagenase IV/Dispase solution for a minimum of a minimum of 3 h at 37° C. Cells were passed by 70 μm strainer and red blood-cells lysis was carried out. Isolated cells containing mainly tumor cells were plated and grown in DMEM growing media. Following continuous media replacement, viable cancer cells remained attached to culture plates and kept growing in culture, while stroma and cell debris were washed.

### Isolation of Murine Brain Stromal Cells

Brains from 6-10 week-old male C57BL/6 mice (Envigo CRS, Israel) were harvested, chopped and incubated in rotation with Collagenase IV/Dispase II solution for 50 min at 37° C. Red blood-cells lysis was carried out followed by Percoll gradient for myelin separation. Cell suspension was then incubated with CD11b or CD31 microbeads for microglia or endothelial cells separation, respectively, and placed in MACS MS magnetic columns. Murine pericytes were isolated using anti-AN2 microbeads combined with anti-CD144 microbeads. The remaining cells collected following beads separation were plated and grown in complete astrocyte medium for a week. Culture enrichment of astrocytes was evaluated for cell type and purity by FACS (GLAST-APC antibody (Miltenyi Biotec, 5 μl/1×10⁶ cells)).

### Swelling

To determine the swelling ability and the time required for swelling equilibrium, kinetic experiments were performed with fibrin 3D-bio-ink as previously described (27). Briefly, fibrin 3D-bio-ink samples were prepared in the absence or presence of GL261 or PD-GB4 cells (1×10⁶ cells/ml). The samples were placed on a stainless-steel grid submerged in a 6-well plate containing 15 ml DMEM to simulate sink conditions, and were incubated at 37° C., 5% CO₂. The swelling kinetics was determined gravimetrically; each fibrin 3D-bio-ink sample was weighed at scheduled time intervals after gently removing excess water from the grid using a Kimwipe® and returned immediately to the well plate. The swelling percentage at each time interval was calculated as follows:

$$\% Swelling = 100 \% \cdot \frac{w - {w}_{o}}{{w}_{o}}$$

Where w₀ is the initial weight of the fibrin 3D-bio-ink sample, right after crosslinking is ended and before swelling and w is the weight of the sample at any other time.

The w₀ refers to the original dimension at the time of fibrinogen polymerization, and not to the dimension of the dry state of the bio-ink since it mimics better the experimental setup that the bio-ink has to tackle. Swelling at equilibrium of polymeric fibrin 3D-bio-ink was estimated from the swelling at the last time point (day 28). All swelling experiments were performed using 6-8 samples per group and the experiments were repeated in three independent times.

### 3D Metabolic Activity Assays

GB cells, hAstro, mAstro, hMG and osteosarcoma cells were cultured in fibrin 3D-bio-ink at different cell densities and gelatin concentrations. Cell viability and growth rates up to 6-7 weeks in fibrin 3D-bio-ink, were evaluated using PresoBlue® Cell Viability Reagent. Cells were counted using Countess™ Automated Cell Counter (Invitrogen). To evaluate the growth curve of GB cells (GL261, T98G, U-87MG, U373, PD-GB1 and PD-GB4), the initial concentration was 1×10⁶ cells/ml in DMEM. To evaluate the growth curve of human primary cells, the initial cell concentrations were 1×10⁵ cells/ml up to 2×10⁶ cells/ml in astrocytes and microglia medium, respectively. To evaluate the growth curve of cells grown in co-culture, GB cells (PD-GB4, GL261, PD-GB1 and U-87MG-D) and astrocytes (hAstro or mAstro) were seeded at the final concentration 2×10⁶ cells/ml at a ratio 1:1 and 1:2 and grown in mixed medium (1:1 ratio) of DMEM and astrocytes medium. To evaluate the growth curve of fast-growing and dormant GB cell types (T98G and U-87MG) in fibrin 3D-bio-ink, cells were seeded at the concentration of 1×10⁶ cells/ml, while osteosarcoma cells (Saos-2) were seeded at the concentration of 1×10⁷ cells/ml in DMEM.

The 3D-bio-ink samples were placed in 500 μl PrestoBlue® in 24 well plate and incubated at 37° C. for 30 min or up to 4 h (depending on the cell type) for growth curve experiments. Three samples of 100 μl from each fibrin 3D-bio-ink sample were taken to a 96 well plate and measured the fluorescence by Excitation: 545 nm, Auto Emission Cutoff (590 nm), Emission: 600 nm.

Measurements were averaged to each sample, a control measurement of a clear fibrin 3D-bio-ink were subscribed and the final result was normalized to the values obtained at the first day of incubation. The fibrin 3D-bio-ink samples were washed with PBS for 15 min at 37° C. and then incubated in fresh cell medium. Each group contained 4-6 samples and the experiments were repeated in three independent times.

### Generation of Fluorescently Labeled Cell Lines

Cells were labeled with pQC-mCherry retroviral particles as previously described (25). Briefly, GL261, U-87MG, T98G, PD-GB3 and HUVEC were labeled with mCherry. Cells were selected for stable expression 48 h following infection by puromycin (2-3 μg/ml, TOKU-E, Singapore). hAstro and hMG cells, patient-derived cells (PD-GB1) and Saos-2 were labeled with green fluorescent protein (GFP). GFP positive cells were then selected using G418 (0.5 mg/ml for Saos-2, 2500 μg/ml for hAstro and for hMG; TOKU-E, Singapore) for 2 weeks. Human pericytes and patient-derived cells (PD-GB4) were labeled with near-infrared fluorescent protein (iRFP) and positive cells were then selected using hygromycin (2.5 mg/ml for PD-GB4 and 250 μg/ml for human pericytes, Sigma-Aldrich, Israel) for 2 weeks. PD-GB4 were also labeled with Azurite and positive cells were selected using hygromycin (250 μg/ml) for 2 weeks. All retroviral infected cells showed over 90% positive fluorescent labelling.

### Imaging and Image Analysis

To evaluate the viability of the labeled cells in the cell-laden bio-ink, fibrin 3D-bio-ink samples were prepared on a 35 mm Petri dish with a bottom grid glass (ibidi®, Gräfelfing, Germany). Media was replaced every other day. Fluorescent images were obtained using SP8 confocal microscope (Leica, Wetzlar, Germany) or with EVOS FL Auto cell imaging system (ThermoFisher Scientific). 3D projections and Z-stacks were generated using manual and automated processes in Leica and Imaris (Imaris 8.4.1, Bitplane Scientific Software). Each group contained 3 samples and the experiments were repeated in three independent times.

### Frozen OCT Fibrin 3D-Bio-Ink Samples Fixation

Fibrin 3D-bio-ink samples (day 7 or 14 after cell seeding) were incubated with 4% Paraformaldehyde (PFA) for 4 h, followed by 0.5 M of sucrose (BioLab) for 1 h, and 1 M sucrose overnight. The samples were then embedded in optimal cutting temperature (OCT) compound (Scigen) on dry ice and stored at −80° C.

### Immunostaining

Fibrin 3D-bio-ink samples embedded in OCT were cryo-sectioned into 5 μm thick sections by a cryostat (Leica). Sections were stained by hematoxylin and cosin (H&E) and for the relevant markers using antibodies. Immunostaining was performed using BOND RX autostainer (Leica). Briefly, slides were incubated with goat serum (10% goat serum in PBS+0.02% Tween-20+0.02% Gelatin) for 30 min to block non-specific binding sites. Slides were then added with Rabbit anti mouse/human GFAP, rabbit anti-mouse IBAI and mouse anti-human P-selectin (R&D Systems, 1:20 dilution). Following 1 h incubation, slides were incubated with the following secondary antibodies for an additional 1 h: Goat anti-rabbit Alexa Fluor® 488 for GFAP and for

IBA1, while goat anti-mouse Alexa-594 for human P-selectin and for muse P-selectin. They were then washed and treated with ProLong® Gold mounting with DAPI or Hoechst 33342 before being covered with coverslips. Images were recorded using the EVOS FL Auto cell imaging system.

### Invasion in 3D-Bio-Ink

PD-GB4 iRFP-labeled were casted into 96 glass bottom well plates (Cellvis, California, USA) in a core tumor at a density of 1×10⁷ cells/ml and surrounded with 1×10⁶ cells/ml un-labeled hAstro. Experiments were done in a medium mixture at 1:1:1 ratio of DMEM, astrocytes medium and microglia medium. For invasion estimation of dormant versus fast-growing cell types, mCherry-labeled U-87MG and T98-G human GB cells (2×10⁶ cells/ml) were plated in a glass bottom 96-well plates in the core of fibrin 3D-bio-ink. The surrounding fibrin 3D-bio-ink was an empty, clear fibrin 3D-bio-ink. Experiments were done in a medium mixture at 1:1:1:1:1 ratio of DMEM, astrocytes medium, microglia medium, pericytes medium and Endo-Gro medium. Cell invasion was monitored for 2-5 weeks using EVOS FL Auto cell imaging system and quantified by ImageJ software, presented here as relative light units (RLU). Cells invasion was normalized by comparing the size of the cell core at day 1 (polygon drawing shape) up to day 14. 2 weeks. Each group contained 6-12 samples and the experiments were repeated in three independent times. For the evaluation of proliferation in the presence of P-selectin inhibitor (SELPi), mCherry-labeled U-87MG-F and T98G-F cells generating fast-growing tumors or iRFP-labeled PD-GB4 cells (4×10⁶ cells/ml) were seeded in a glass bottom 96-well plate in the core of fibrin 3D-bio-ink. The surrounding fibrin 3D-bio-ink was an empty, clear fibrin 3D-bio-ink. Cells were then treated with 0.5 M KF38789 which is a selective inhibitor of P-selectin (SELPi) which were chosen to serve as a small molecule drug. It selectively inhibit SELP binding in lymphocytes at the half maximal inhibitory concentrations (IC₅₀) of 2 μM. P-selectin (SELP) functions as a cell adhesion molecule on the surfaces of cells which binds carbohydrates (18). It is also involved in tumor cell binding and promote their invasion by supporting a permissive metastatic microenvironment and SELP is highly expressed in GB (28). Control groups were treated with DMSO vehicle at the corresponding volume percentage (0.2% v/v), prepared in a medium mixture at 1:1:1:1:1 ratio of DMEM, astrocytes medium, microglia medium, pericytes medium and Endo-Gro medium. Proliferation was quantified during time with ImageJ software by RLU. Each group contained 6-12 samples and the experiments were repeated in three independent times.

### Scanning Electron Microscope (SEM)

For SEM analysis, acellular and cell-laden fibrin 3D-bio-ink sample were prepared and incubated for 14 days at 37° C., 5% CO₂. Cell-laden samples were prepared using PD-GB4 cells (1×10⁷ cell/ml) alone or in co-culture with hAstro (1×10⁷ cell/ml) and hMG (1×10⁶ cell/ml). Fibrin 3D-bio-ink samples were then removed from the culture medium after 14 days, washed in PBS and fixed with 2.5% v/v glutaraldehyde (Sigma) in PBS over night at 4° C. The samples were snap frozen with liquid nitrogen and lyophilized. The same procedure was done for both healthy and tumorous (GL261 intracranially injected cells, 15-20 days post implantation) brain hemispheres of C57BL/6 mice. Samples were stored under vacuum in a desiccator before sputter-coating with gold (˜20 nm). SEM images were taken using Quanta 200 FEG Environmental SEM (FEI, OR, USA) at high vacuum and 10.0 KV. Lengths and pore size were measured using ImageJ software.

### Preparation of the Vascular Bio-Ink

Pluronic F127 is a biologically inert triblock co-polymer based on poly-ethylene oxide (PEO) and poly-propylene oxide (PPO), which exhibits reversible thermal gelation and a lower critical solution temperature (LCST) behavior. At lower temperatures, Pluronic F127 solution is liquid, and upon heating to room temperature it transforms into a gel. 40% w/v Pluronic F127 was dissolved in double distilled water (DDW) using an overhead mechanical stirrer at 4° C. The solution was stored at 4° C. Prior to use, 2000 U/ml thrombin solution was added to create the fugitive vascular bio-ink of final 38% w/v Pluronic F127 and 100 U/ml thrombin. The vascular bio-ink was loaded into a 30 ml syringe at 4° C. and centrifuged to remove air bubbles.

### Fabrication of 3D-Bioprinted Vascularized Tumor Model

Printing formulation of fibrin 3D-bio-ink (composed of 1% w/v fibrinogen and 6% w/v gelatin) The enzymes (thrombin and TG) are not added to the printing formulation to avoid cross-linking in the printer. The printing formulation was mixed with PD-GB4 cells labeled with Azurite (1×10⁶ cells/ml), hAstro labeled with GFP (1×10⁶ cells/ml) and hMG cells (1×10⁵ cells/ml). The mixture forming the GB-bio-ink was loaded in a syringe (Nordson, California, USA) and tapered with a needle tip (Nordson) of varying size attached via a luer-lock. The GB-bio-ink was cooled to 4° C for 15 min and then loaded into the 3D printer's cartridge. Temperature was set to 24° C. and held for 15 min prior to 3D-bioprinting initiation. 3D structures were printed using a 3D-Bioplotter® (Manufacturer series, EnvisionTEC®, Gladbeck, Germany) equipped with five independent ink cartridges. Before each 3D-bioprinting, the nozzles were calibrated to determine their respective X, Y and Z offsets. Following calibration, 6 to 8 layers were printed on a thin coverslip, framed by a polydimethylsiloxane (PDMS) (Sylgard™ 184, Dow®, Michigan, USA) gasket, creating the bottom platform onto which vasculature structure could be printed. Cell-laden bio-inks were printed for a time period up to 2 h to prevent cell death. The printed layers were left to dry for up to 1 h. Casting formulation of fibrin 3D-bio-ink (composed of 1% w/v fibrinogen and 6% w/v or 12% w/v gelatin) was mixed with Azurite-labeled PD-GB4 cells (1×10⁶ cells/ml), GFP-labeled hAstro (1×10⁶ cells/ml) and hMG (1×10⁵ cells/ml). In another experiment, the mixture forming the GB-bio-ink was casted on a thin coverslip, framed by a PDMS gasket, creating the bottom platform onto which vasculature structure could be printed. Cell-laden fibrin 3D-bio-ink constructs were left to fully crosslink for up to 2 h. Regardless of the method of dispensing the first layer(s) of the tumor (whether by printing or casting), the vascular bio-ink composed of Pluronic F127 38% w/v and thrombin 100 U/ml was loaded onto a printer cartridge (Nordson) at 4° C., warmed to 29° C., and printed using a 0.25 mm needle tip attached via a luer-lock. 3D structures were printed with the vascular bio-ink according to a bioengineered design of vasculature, created with Rhino 6® 3D modeling software (Rhinoceros®). Immediately after the vascular bio-ink 3D-bioprinting was completed, connectors (Darwin microfluidics, Paris, France) were inserted to the inlet and outlet positions of the PDMS gasket to allow future flow by peristaltic pump, and a fresh formulation of the fibrin 3D-bio-ink with PD-GB4 and microenvironment cells was casted, covering the printed vasculature and filling the PDMS frame completely. The sample was then covered with additional coverslip glass and sealed in a metal frame (creating the perfusable chip), self-designed for optimal noninvasive monitoring of fluorescent signals and incubated at 37° C. for a minimum of 3 h until complete crosslinking is achieved, reaching the physiological stiffness of the brain. To liquefy the fugitive Pluronic F127 bio-ink, the sample was cooled to 4° C. Cold PBS was then injected into the mold's inlet by applying positive pressure, while applying negative pressure through the outlet leaving the model with the desired 3D-bioprinted lumens. Following Pluronic F127 wash, fibronectin (100 μg/ml) was injected into the lumens' inlet and incubated in rotation for 3 h at 37° C. to prime the vasculature wall and create an adherable interface. Next, a mixture of mCherry-labeled HUVEC (8×10⁶ cells/ml) and iRFP-labeled human microvascular brain pericytes (2×10⁶ cells/ml) (named vascular bio-ink) at 4:1 ratio was injected into the vessel and incubated for 2 h. Cell seeding steps were repeated 4 times, and each time sample incubation positioning was rotated by 90 degrees to maximize cell attachment area. The model was then incubated in rotation overnight at 37° C. to allow cell attachment to the lumens' walls with a full coverage of the lumen. The next day the sample was connected to a peristaltic pump (EBERS, Zaragoza, Spain), incubated at 37° C. and perfused with medium mixture of all the cells in the samples at a 1:1:1:1:1 ratio (DMEM, astrocyte medium, microglia medium, pericyte medium and EGM-2) for 5 days. After confluent cover was validated by confocal imaging, 1 and 0.1 mg/ml 70 kDa dextran-FITC was perfused through the vasculature using a syringe pump (Braintree scientific, Braintree, Massachusetts, USA) at a flow rate of 25 μl/min. Dextran-FITC flow-through was imaged by EVOS FL Auto cell imaging system at 20 sec intervals. 3D-printed GB models used for dextran-FITC perfusion were created with unlabeled cancer and stromal cells (hAstro and hMG).

Creating the metastatic model: A simplified vascularized 3D model of breast cancer brain metastasis was created using a 23G needle inside the perfusion chip. Brain metastatic human breast cancer cells MDA-MB-231, astrocytes, and microglia were mixed with the fibrin 3D-bio-ink in similar conditions as the GB microenvironment according to the present calibrations (cell type ratio was 1:1:0.1 respectively). Moreover, functional blood vessels prepared using human endothelial cells and human microvascular brain pericytes in the same ration of 4:1 following the same crosslinking and seeding protocol.

### 2D Proliferation Assays

For growth curve evaluation, U-87MG and T98G human GB cells dormant and fast-growing tumor cells (29) (25,000 cells/well) were seeded onto 24-well plates in complete DMEM and incubated for 72 h (37° C.; 5% CO₂). For the comparison of Saos-2 cell types growth curves in 2D culture, 10,000 cells/well were cultured in DMEM following a daily incubation with PrestoBlue® reagent for 4 h. For proliferation evaluation in the presence of SELPi, U-87MG and T98G human GB cells generating dormant and fast-growing tumors, or PD-GB4 cells (25,000 cells/well) were plated onto 24-well plate in complete DMEM and incubated for 24 h (37° C.; 5% CO₂) to allow cell attachment. Cells were then treated with either 0.5 μM SELPi or with DMSO vehicle at corresponding % v/v of 0.2% v/v, prepared in medium mixture at 1:1:1:1:1 ratio of DMEM, astrocytes medium, microglia medium, pericytes medium and Endo-Gro medium, for 30 min. All wells were washed twice with PBS and incubated further with fresh medium mixture. Cell proliferation was monitored by IncuCyte® Zoom Live cell analysis system (Essen Bioscience), while the first images were taken right before the treatment. Each group was assayed in triplicates and the experiment was repeated in three independent times. Proliferation of cells was normalized to time 0 and presented as percent (%) of cell confluence.

### Wound Healing Assay

U-87MG and T98G human GB cells (7.5×10⁴ cells/well) were plated onto 96-well plate (ImageLock, Essen BioScience, Hertfordshire, United Kingdom) in DMEM medium. A scratch was performed using a WoundMaker™, followed by two washes with PBS. Cells were then treated with either 0.5 μM SELPi or with DMSO vehicle at corresponding % v/v. Wound density % was monitored and quantified by Incucyte® ZOOM (Essen BioScience). Each treatment was assayed in triplicates and the experiment was repeated in three independent times.

### Animal Models

For gene expression analysis, mCherry-labeled murine GL261 GB cells (5×10⁴) were stereotactically implanted into the striatum of 7 week-old male C57BL/6 immunocompetent mice (n=5, Envigo CRS), prior anesthesia using ketamine (150 mg/kg) and xylazine (12 mg/kg) injected intraperitoneally (IP). Mice were euthanized on day 14 post injection and the brains were resected for further evaluation of gene expression. For the establishment of human GB dormancy model, T98-G cells (1×10⁶) were injected subcutaneously into the flank of 6-8 weeks old male SCID mice (Envigo CRS). Tumor volume was monitored by caliper measurement (width²×length×0.52). Body weight was monitored q.o.d.

### Flow Cytometry for P-Selectin Expression

For 2D culture samples, GB cells (1×10⁶ cells/10 cm² petri) were grown in DMEM medium for 24 h, then scraped with 1 ml PBS and centrifuged for 5 min at 4° C., 2000 rpm. The pellet was resuspended in FACS buffer (PBS supplemented with 0.5% Bovine Serum Albumin (BSA) and 0.5 mM EDTA (Sigma Aldrich)) and cells were counted using Countess™ Automated Cell Counter (Invitrogen). For 3D-bio-ink samples, GB cells (1×10⁶ cells/ml) were grown in fibrin 3D-bio-ink for 10 days in DMEM medium. Then, the fibrin 3D-bio-ink was digested with collagenase II (600 U/ml, 1.5 h) and filtered through a 70 μm mesh to avoid undigested polymeric clots or particles. The suspension was centrifuged for 5 min at 4° C., 2000 rpm and supernatant was discarded. Murine GL261 cells were incubated with FITC rat anti-mouse anti- P-Selectin or FITC rat IgGI isotype control, for 1 h on ice at a concentration of 3 μl per 1×10⁶ cells. After incubation, FACS buffer was added to the pellet and cells were washed twice by centrifugation for 5 min at 4° C., 2000 rpm. Human cells (PG-GB4, T98G and U-87MG) were first incubated with non-labeled primary antibody (Mouse anti-human E/P selectin or Mouse IgG1 I.C.) for 1 h on ice. Then, FACS buffer was added to the pellet and cells were washed by centrifugation for 5 min at 4° C., 2000 rpm. Cells were then incubated with mouse-IgG & BP-CFL 488 secondary for 1 h on ice, followed by 2 washes as described above. Unstained sample and the corresponding isotype control were used as internal control for unspecific antibodies epitope binding. Fluorescent intensity was evaluated using Attune NxT Acoustic Focusing Flow Cytometer (Thermo-Fisher Scientific) and analyzed by the Kaluza 1.3 software (Beckman Coulter). Each group contained 4-8 bio-ink samples which were pooled after the bio-ink digestion and the experiments were repeated in three independent times. For 3D-bio-ink samples containing PG-GB(1-4) cells, the same procedure was used with 1×10⁷ cells/ml (not shown).

### RNA Sequencing

To evaluate changes in gene expression in the fibrin 3D-bio-ink compared to 2D culture and in-vivo models, RNA seq analysis was performed. For 2D culture and 3D-bio-ink models, a murine penta-culture composed of GL261 mCherry-labeled and freshly-isolated murine primary astrocytes, microglia, pericytes and endothelial cells was grown on a 2D petri dish, or in fibrin 3D-bio-ink GL261 cells were seeded at the concentration of 1×10⁶ cells/ml (in fibrin 3D-bio-ink or in 2D culture) at the ratio of 1:1:2:0.1:0.1 creating the penta-culture. For the in-vivo GB model, mCherry-labeled GL261 tumor cells were sorted from tumor-bearing mice at day 14 after stereotactic implantation. 3D-bio-ink was cut into 1-2 mm fragments, digested with 600 U/ml collagenase II for 3 h and filtered with a 70 μm mesh prior RNA extraction. Total RNA was then isolated from all samples using EZ-RNA II total RNA isolation kit (Biological industries, California, USA), following the manufacturer's protocol. RNA quality was evaluated by TapeStation® RNA Assay (Agilent Technologies). Libraries were constructed with the NEBNext® Ultra II Directional RNA Library Prep Kit for Illumina® (#E7760, NEB) using manufacturer's instructions. Twenty-five to 75 ng RNA was amplified using 14 cycles of PCR. Final quality was evaluated by TapeStation® DNA HS Assay (Agilent Technologies). Equimolar pooling of libraries were performed based on Qubit® values and loaded (in triplicates) onto an Illumina® NextSeq 500 platform (Illumina, California, USA) in order to evaluate changes in gene expression between the tested groups.

### RNA seq Analysis

Data analysis was aligned to mice genome. RNA-seq reads were aligned using Kallisto (30) to mouse genome version mm10, and expression levels were calculated using RSEM (31), followed by further processing using the Bioconductor package DESeq2 1.24.0 in R (32). The data were normalized using TMM normalization, and differentially expressed genes were defined using the differential expression pipeline on the raw counts with a single call to the function DESeq (FDR-adjusted P value<0.05). For global transcriptome quantification and comparison between the samples (2D culture, fibrin 3D-bio-ink and in-vivo,) PCA analysis was performed and the Euclidean distance was calculated between each pair of transcriptional profiles.

### Temozolomide (TMZ) Resistance Evaluation in 2D Versus 3D

The resistance of patient-derived cells to TMZ (LIXIN, China) was assessed in fibrin 3D-bio-ink and in 2D culture. Patient-derived cells were seeded in fibrin 3D-bio-ink at 1×10⁷ cells/ml density to match resemble a very dens GB tissue, in different TMZ concentrations (2000 μM -0.01 μM) for 7 days. For the 2D culture TMZ resistance evaluation 20,000-40,000 cells/well were seeded in 24 well plate in fresh DMEM. We replaced the media to media with different TMZ concentrations (10 μM -0.01 μM) for 3 days. Then, the 3D-bio-ink samples were placed in 500 μl PrestoBlue in 24 well plate and incubated at 37° C. for 3 h.

### Statistical Methods

Data is expressed as mean±standard error of the mean (SEM). Statistical significance was determined using an unpaired two-sided student t-test when comparing between two groups and analysis of multiple comparisons (ANOVA test) when comparing more than two groups. P<0.05 was considered statistically significant. All experiments were performed in 3 biological replicates with cells from different batches to ensure reliable and repetitive results. Statistical analysis was performed using GraphPad Prism 8.

### Data Availability

The sequence data generated in this study have been deposited in the Gene Expression Omnibus (GEO) and are accessible through the GEO Series accession number GSE178371.

### EXAMPLE 1

### Fibrin 3D-Bio-Ink Formulation and Characterization

A polymeric bio-ink was produced based on fibrinogen and gelatin which can provide a supporting 3D structure for GB cells and their microenvironment. First, the present inventors evaluated the effect of the addition of the crosslinker, Thrombin (Th), on the gelation time of a mixture of gelatin, fibrinogen and transglutaminase (TG) (6% w/v, 1% w/v and 3% w/v, respectively) at different Th concentrations (0 U/ml, 0.5 U/ml, and 1 U/ml) via time sweep rheological test. Th induced rapid crosslinking and reached higher elastic modulus (G′) in a dose-dependent manner until complete crosslinking (FIG. 1A). Of note, a mixture of fibrinogen with TG alone and serial gelatin concentrations, without Th, was not sufficient to create a matrix with the appropriate tissue like stiffness (not shown). Furthermore, the present inventors have used lower Th concentration (0.5 U/ml) to ensure homogenous mixing of the 3D-bio-ink before casting. Moreover, Young's modulus (E) of the created fibrin 3D-bio-ink was evaluated by calculating and comparing the slope of a strain-stress curve, obtained by performing a compression test of several 3D-bio-ink formulations at constant fibrinogen (1% w/v), Th (0.5 U/ml) and TG (3% w/v), and gelatin at serial concentrations (3% w/v, 6% w/v and 12% w/v). It was found that the stiffness can be controlled by varying the final concentration of the gelatin, to generate the desired mechanical property of GB tumor which is compatible with cell growth. Young's modulus of 24.5+2.6 kPa was achieved when the final gelatin concentration was 6% w/v (FIG. 1B). The present results are in agreement with a previous study conducted on U-87MG xenografts, reporting a tumor Young's modulus of 26.6 kPa (33). Importantly, the Young's modulus of the cell containing formulation (patient-derived GB cells PD-GB4 or murine GB cell type GL261) remained constant for 28 days. Another important physico-chemical parameter of a bio-ink is its swelling behavior, which determines the accuracy of the 3D-bioprinted final structure. Furthermore, a kinetic swelling study was performed in the absence or in the presence of murine GB cells at different gelatin concentrations. Fibrin 3D-bio-ink samples containing 3% and 6% w/v gelatin reached an equilibrium state at around 5% swelling after 28 days, while samples containing 12% w/v gelatin reached an equilibrium state at around 20% swelling indicating the ability of the bio-ink to retain its deposited dimensions (FIG. 1C). These findings are in agreement with previous reports (34). Next, the present inventors set to calibrate the optimal seeding density of different GB models (GL261, human T98G, U373 and U-87MG, as well as PD-GB4) in the fibrin 3D-bio-ink at 6% w/v gelatin. It was found that the initial optimal cell seeding density is 1×10⁶ cells/ml to achieve rapid growth rates. Cells from different origins (murine, human, patient-derived) showed different growth rates in the fibrin 3D-bio-ink. Next, the present inventors set to evaluate the proliferation rate of GL261 and PD-GB4 at different gelatin concentrations at a seeding density of 1×10⁶ cells/ml. Cells at 6% w/v gelatin showed the highest proliferation rates in the fibrin 3D-bio-ink (FIG. 1D). Therefore, it was decided to continue with further investigations using the fibrin 3D-bio-ink at the final concentrations of 1% w/v fibrinogen, 6% w/v gelatin, 3% w/v TG and 0.5 U/ml Th (unless stated otherwise). Diverse cell morphology, as demonstrated by fluorescence imaging of live and fixed fibrin 3D-bio-ink samples, as well as by H&E staining (FIG. 1E), indicated that GB cells in the 3D-bio-ink may interact differently with the bio-ink creating cell-matrix interactions that are unique to each cell type.

### EXAMPLE 2

### Fibrin 3D-Bio-Ink is Suitable for In-Vitro and Ex-Vivo Assays

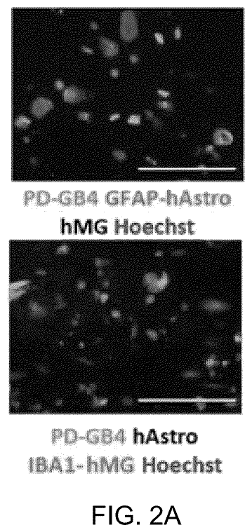

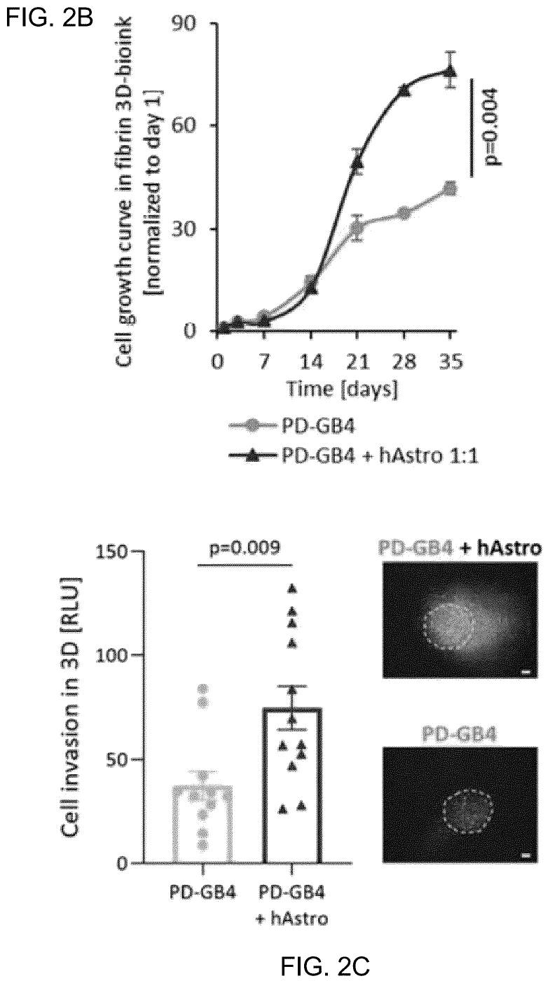

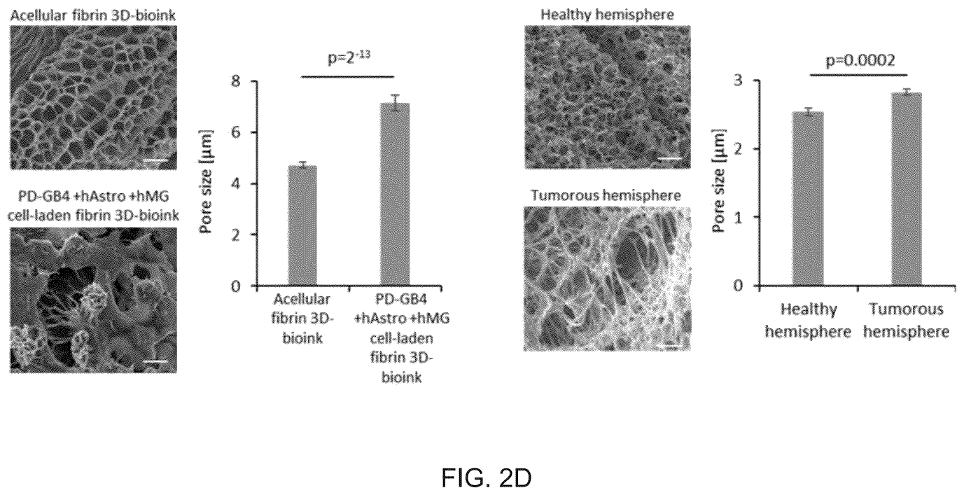

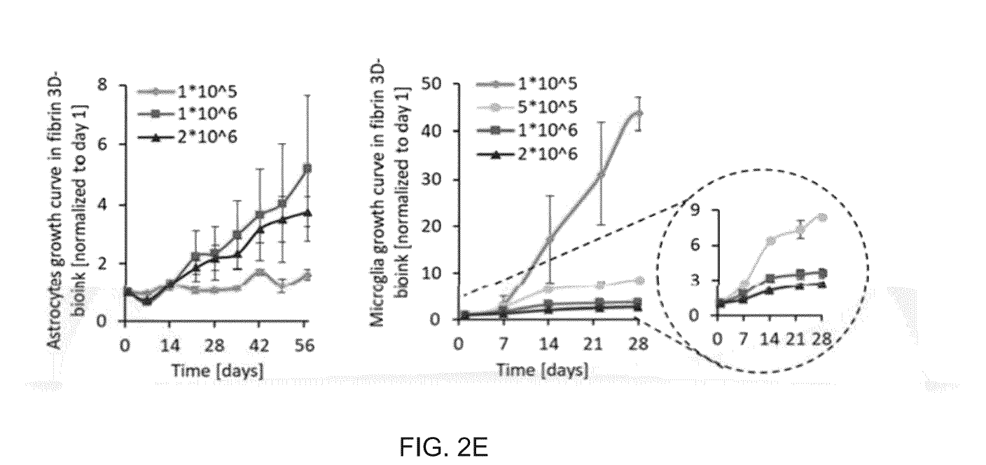

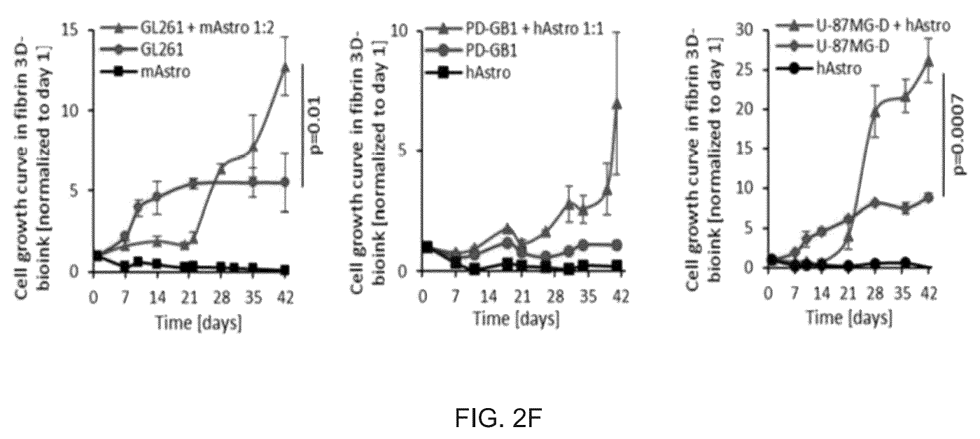

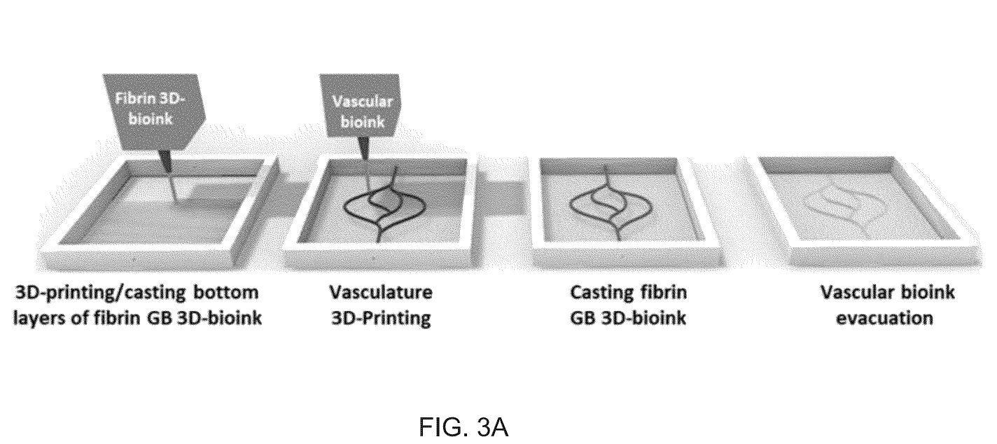

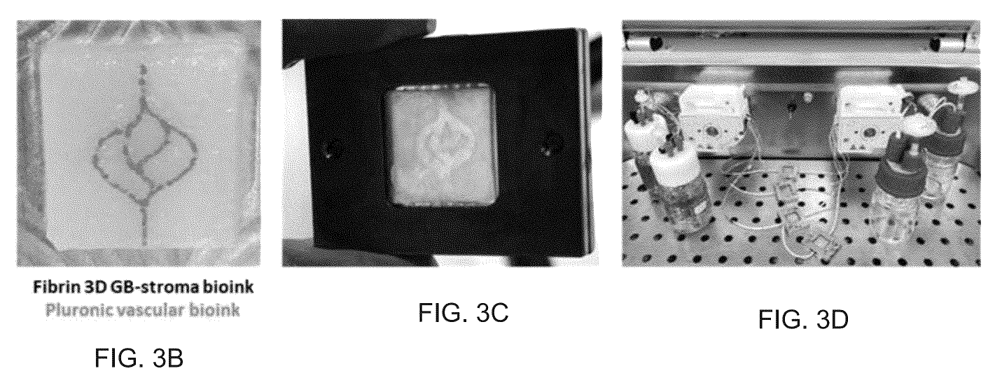

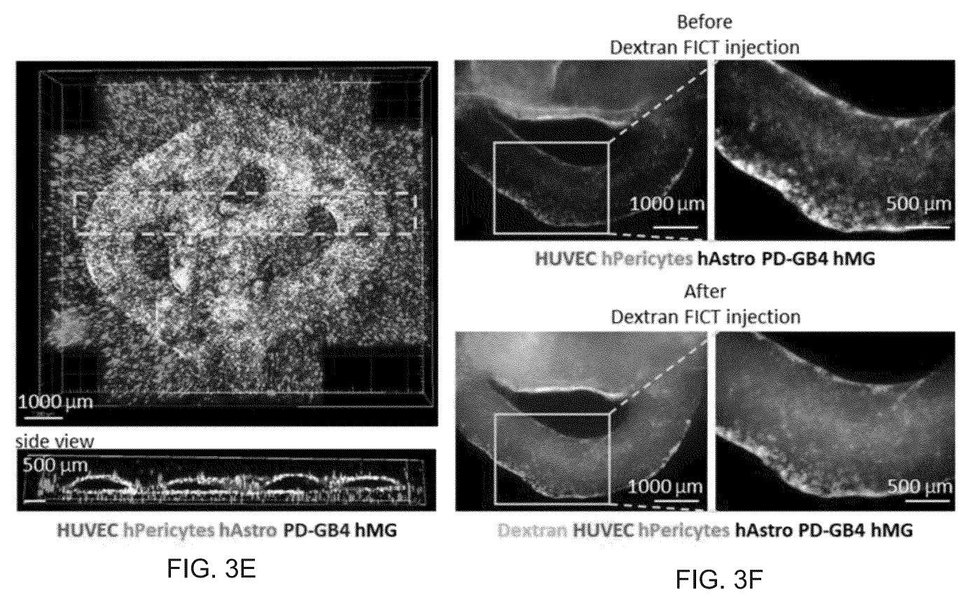

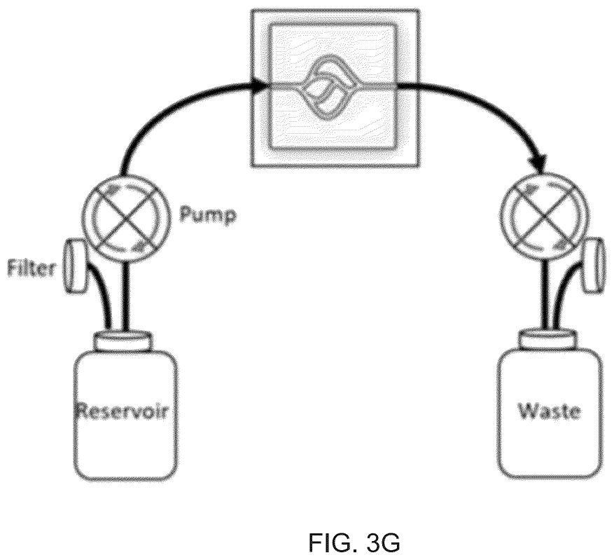

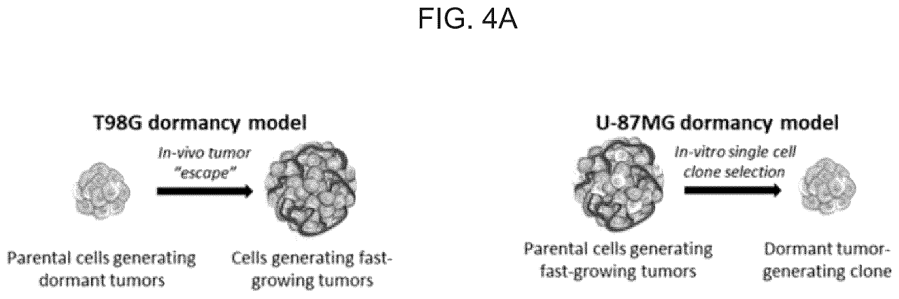

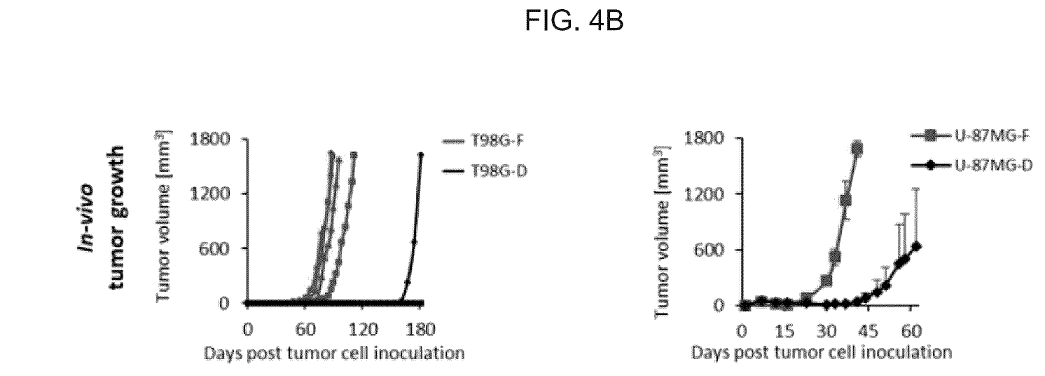

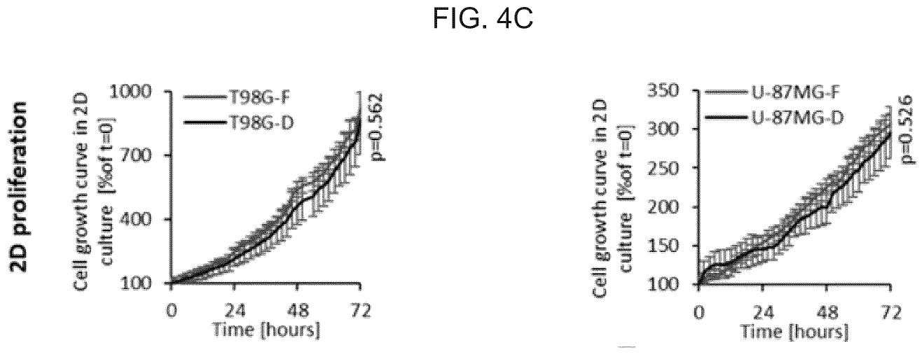

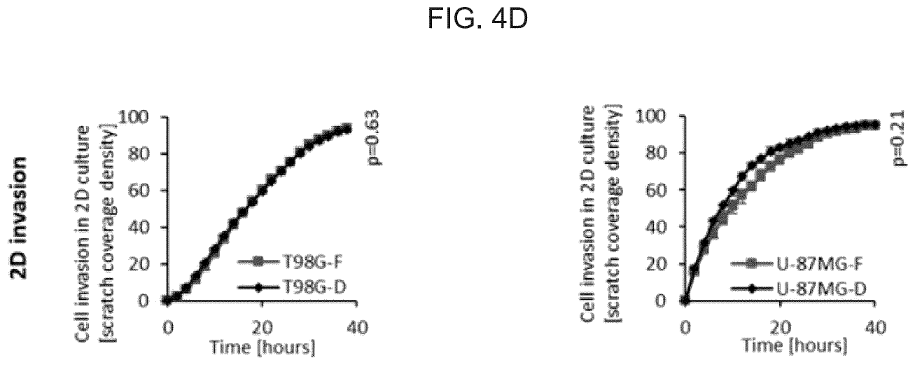

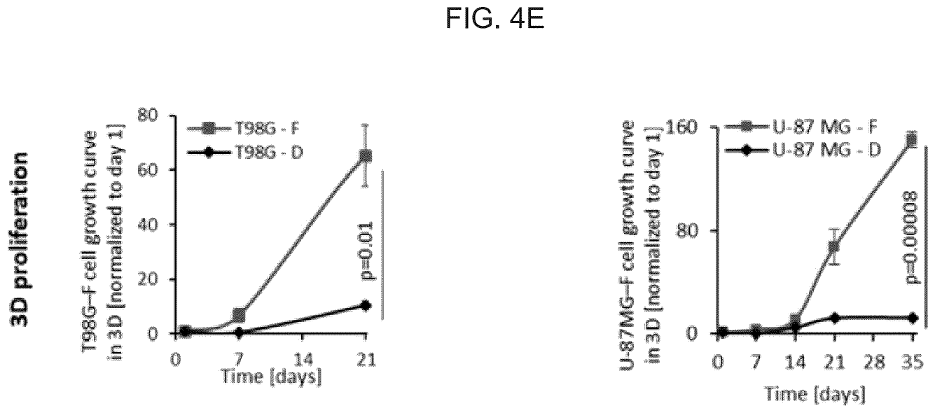

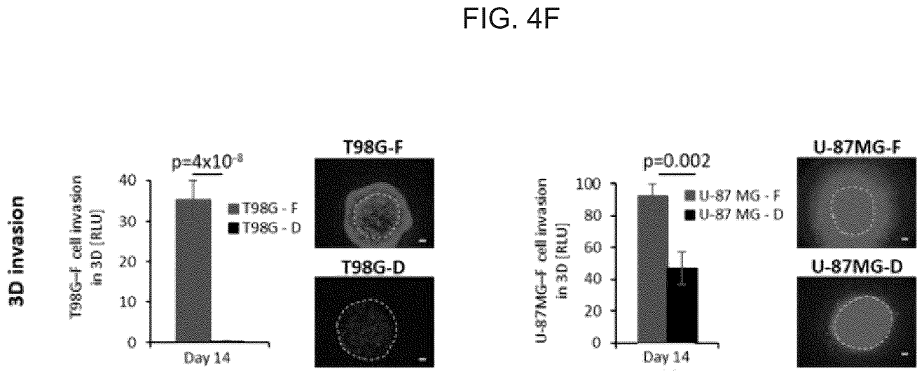

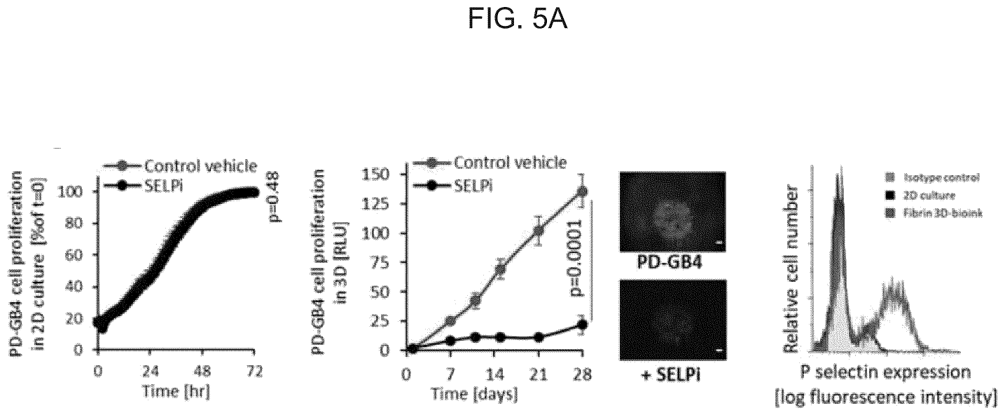

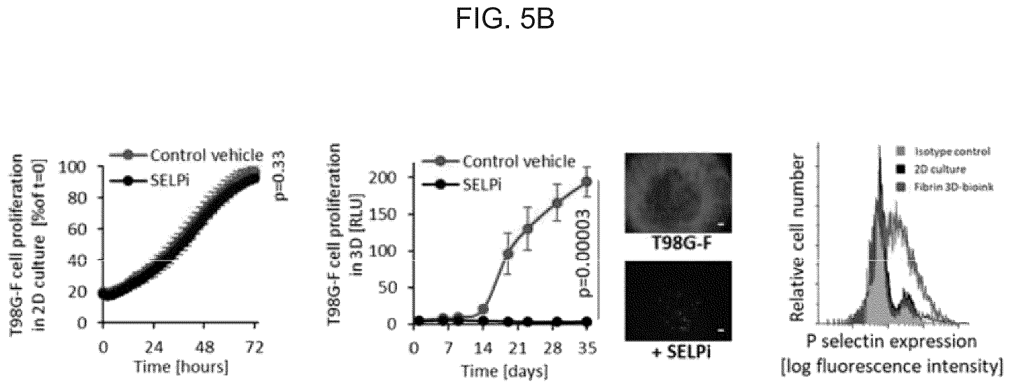

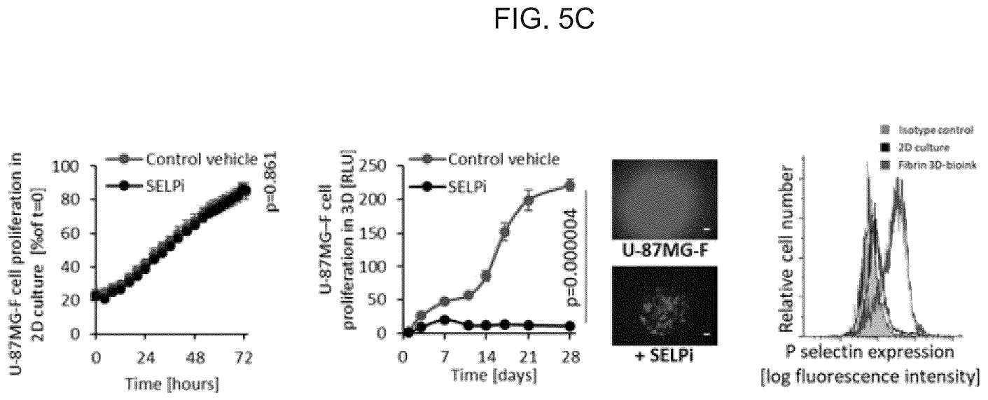

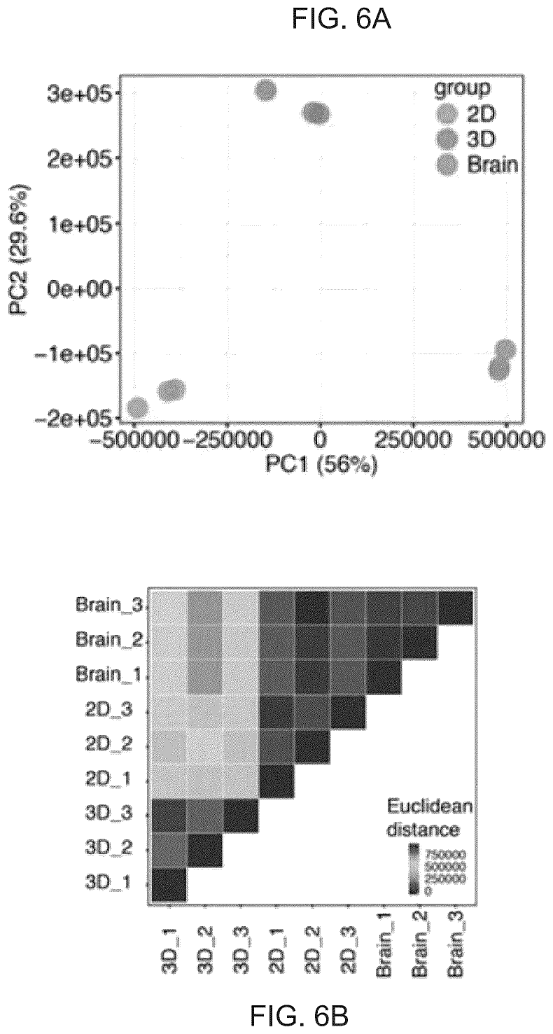

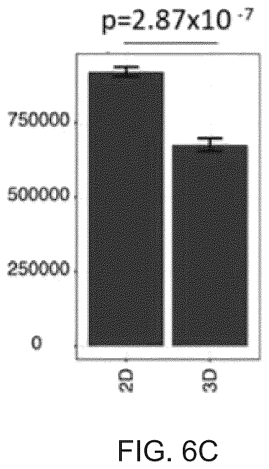

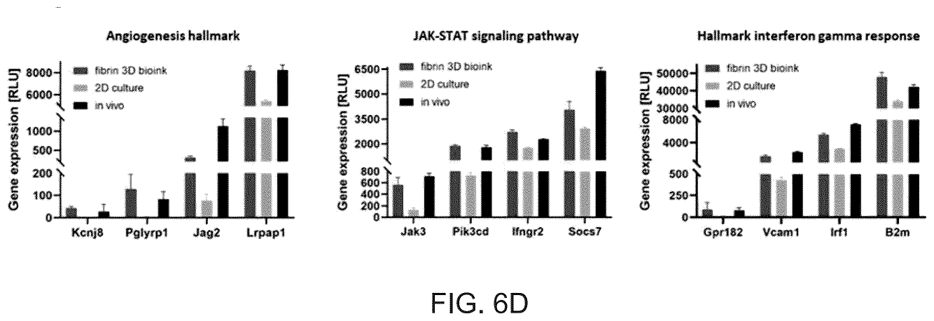

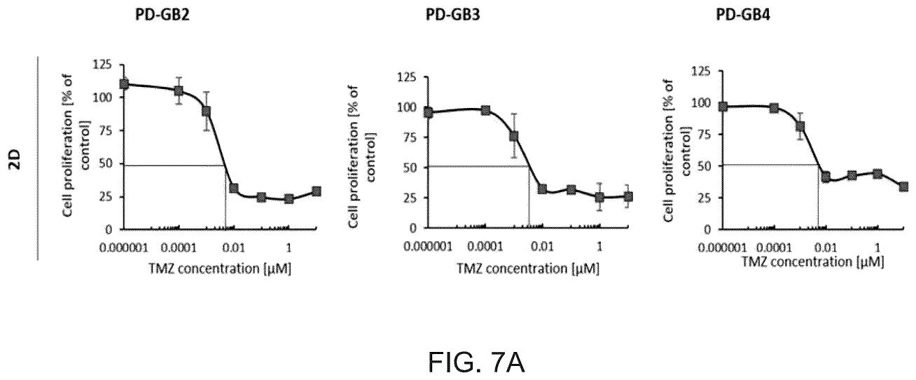

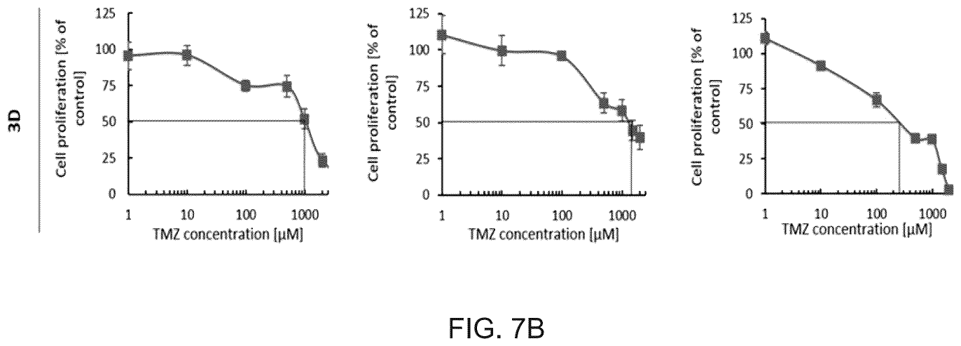

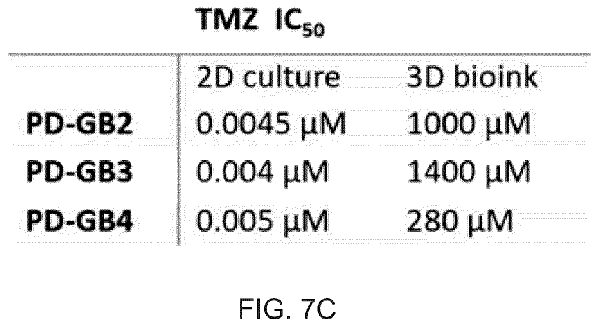

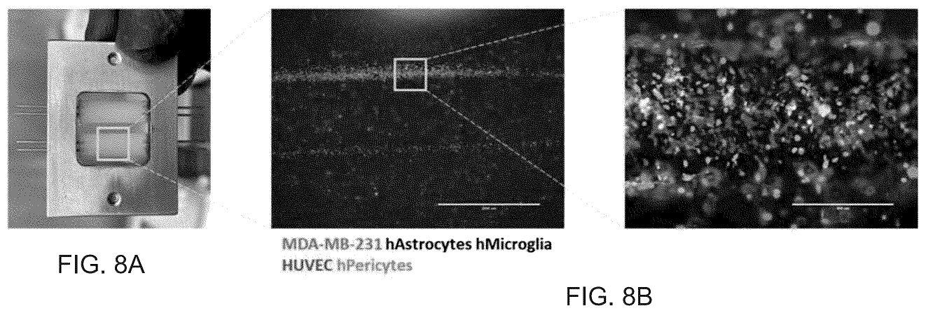

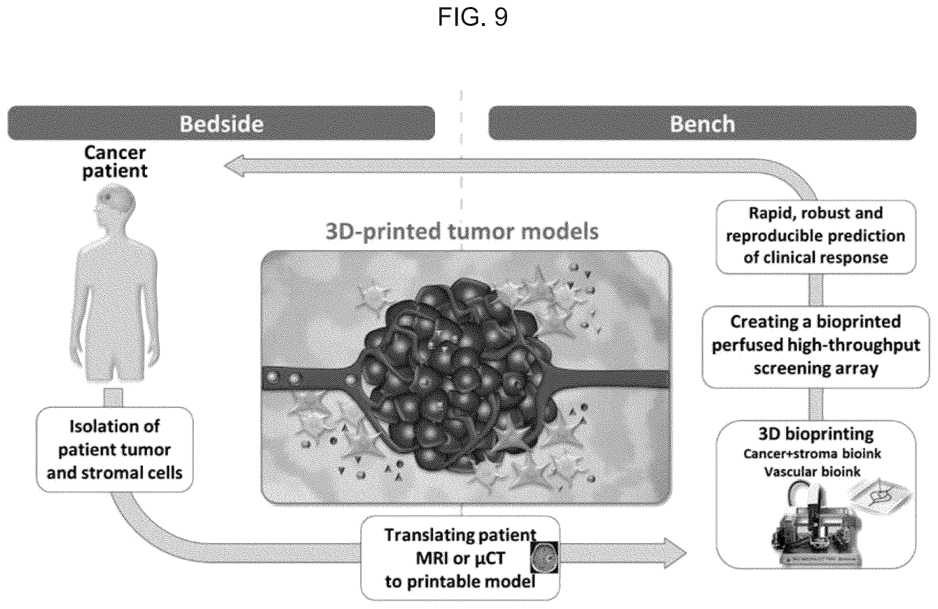

The tumor micro environment (TME) is critical for a proper evaluation of cancer cell growth and response to drugs. Therefore, the present inventos set to recapitulate the complexity of the brain microenvironment by the addition of stromal cells in the fibrin GB 3D-bio-ink. The present inventors evaluated the growth of two of the main cellular components of the brain TME, astrocytes and microglia, at different cell densities according to their abundance in human brain samples; 20-60% of the total number of glial cells in the human brain are astrocytes (35). The growth rate of human primary astrocytes (hAstro) positively correlated with the number of cells seeded in the 3D-bio-ink. On the other hand, microglia cell viability was enhanced when primary human microglia (hMG) cells were seeded in low amount (FIG. 2E). These results showed that the fibrin 3D-bio-ink can support the growth of the brain microenvironment cells over time. Next, PD-GB4 cells were co-cultured together with hAstro and hMG using the fibrin 3D-bio-ink. Expression of hAstro and hMG activation markers (GFAP and IBA1, respectively) was observed in sections of the fibrin 3D-bio-ink 7 days post cell seeding (FIG. 2A). Activation of astrocytes and microglia were similarly detected in ex vivo analysis comparing the stromal activation of murine and human GB tumors versus healthy tissue (36, 37). This suggests that cells grown in the 3D system of herein described have similar biological properties as observed in human GB samples and in mouse models. The addition of hAstro enhanced the growth rate of patient-derived PD-GB4 cells, suggesting that hAstro secrete factors that enhance cancer cell proliferation (FIG. 2B). A similar increase in GB cell growth was observed using 3 additional GB models: GL261, PD-GB1 and U-87MG (FIG. 2F). In addition, it was found that the invasion ability of PD-GB4 was enhanced by 50% towards the outer 3D-bio-ink containing hAstro compared to naive surrounding bio-ink (FIG. 2C). The present inventors then evaluated the structure of the fibrin 3D-bio-ink using scanning electron microscopy (SEM) in comparison to healthy and tumor bearing mice brain tissues from C57BL/6 mice. Acellular fibrin 3D-bio-ink and healthy hemisphere show smaller pores compared to the larger pores observed in the cell-laden fibrin 3D-bio-ink and tumor containing hemisphere. Nonhomogeneous structure and an average pore size of 4.7 μm in diameter was measured in acellular fibrin 3D-bio-ink; therefore, small molecules such as oxygen, nutrients and drug molecules can diffuse freely through the model. The pore size of the GB-bio-ink was significantly increased when PD-GB4 cells were seeded alone or co-cultured with brain stromal cells (hAstro and hMG) in the 3D-bio-ink (7.2-7.1 μm diameter, respectively). The addition of activated and proliferating tumor and stromal cells alters the appearance of synthetic and natural ECM and significantly increase their pore size (p=2×10⁻¹³, t-test). Similarly, the pore size of the tumor containing hemisphere was higher than the pore size in a healthy hemisphere (p=0.0002, t-test). Additionally, the cell-laden fibrin 3D-bio-ink show cellular filopodia towards the surrounding matrix, suggesting the presence of cell-cell and cell-ECM interactions (FIG. 2D).

### EXAMPLE 3

### 3D-Printed Vascularization

Functional vasculature is essential for the growth and function of the tumor cells and their surrounding TME, as it supplies oxygen and nutrients, and essential for the clearance of metabolic excrements [23]. In the absence of perfusable vasculature, cells are unable to remain viable if they are far from a blood vessel beyond the oxygen diffusion distance of approximately 200 μm, leading to the development of necrotic regions. Therefore, the present inventors have engineered a functional vascular included in our GB-bio-ink as illustrated in FIG. 3A. A pattern resembling vascular structure was designed, including curves, branching and anastomosis, using Rhino® 3D modeling software. A GB-bio-ink was created by mixing the fibrin 3D-bio-ink with PD-GB4 cells, hAstro and hMG. The vascular network was created using a fugitive bio-ink, composed of Pluronic F127 and Th, which was printed on top of the 3D printed/casted fibrin 3D-bio-ink according to the bioengineered design (FIG. 3B). After casting another layer of GB-bio-ink on top of the printed vasculature, the vascularized 3D-bioprinted model was sealed in a self-designed metal perfusion chip (FIG. 3C). Following removal of the vascular ink, a mixture of HUVEC and human pericytes was injected directly into the hollow channel and incubated in rotation to allow cell attachment homogeneously to the lumens' walls. The resulting penta-culture, vascularized 3D-bioprinted GB model, was connected to a peristaltic pump to facilitate the formation of the vascular lumen layer under flow for 5 days (FIG. 3D, schematic illustration in

FIG. 3G). Cell arrangement was monitored by fluorescence and confocal microscopy to evaluate the coverage of the hollow channels with endothelial cells and pericytes. The formation of the desired 3D-bioprinted lumens was confirmed (FIGS. 3E). Similar 3D-bioprinting protocol was performed with a penta-culture mixture of cells using a bio-ink prepared at a final gelatin concentration of 6% with PD-GB4 cells (not shown), or PD-GB3 cells (not shown). To evaluate the functionality of the vascular network in the bioengineered GB model, the present inventors created the vascularized 3D-bioprinted model with the same composition of un-labeled cells in the GB-bio-ink to avoid overlapping of fluorescent signals. They perfused the vasculature with 70 kDa dextran-FITC solution using a syringe pump and monitored the flow of dextran-FITC by fluorescent microscopy. The use of the Dextran FITC shows the potential to perfuse the sample through the vessels that are used as channels to transport nutrients and waste into and out of the system, respectively. These perfusable blood vessels offer the possibility to mimic the blood brain barrier allowing the evaluation of the integrity and functionality of the endothelial barrier in the presence or absence of cancer cells (FIG. 3F).

### EXAMPLE 4

### 3D GB Model Resemble the In-Vivo Tumor Settings Better than 2D Models

Previously established and comprehensively characterized are two pairs of GB cell types by generating dormant (“D”) and fast-growing (“F”) GB models (29, 38). The first pair was established from the human T98G GB cell line (T98G-D), which generates small, undetectable tumors following inoculation into mice, that remain dormant for prolonged periods of time until they spontaneously escape and grow. From the “escaped” tumors, a “fast-growing” clone (T98G-F), was isolated which generates tumors rapidly (39). The second pair was established using the aggressive human U-87MG GB cell line (U-87MG-F), from which a dormant tumor-generating clone (U-87MG-D) out of many single cell clones was isolated. Despite the significant differences in their in-vivo growth patterns, these pair of cells display similar characteristics in-vitro showing similar growth rate kinetics when grown in-vitro in 2D culture (FIGS. 4A-B) (29, 38). The present inventors therefore evaluated whether the fibrin 3D-bio-ink was able to mimic in-vivo tumor dormancy or fast growing, using the two models of dormant and fast-growing GB models.

In accordance with previous publications, inoculation of T98G-F cells into SCID mice resulted in tumor growth within two months, while T98G-D remained dormant for up to 5.5 months. Similar tumor growth patterns were observed when U-87MG-F and D were inoculated in SCID mice (FIG. 4B). However, when cultured in 2D monolayer both pairs of dormant and fast-growing cells displayed similar growth rates and invasion capabilities, losing their growth characteristics and differences observed in-vivo (FIGS. 4C-D). Cells grown in the fibrin 3D-bio-ink showed similar tumorigenic characteristics as in the in-vivo settings. Particularly, cell generating fast-growing tumors displayed a significantly higher (p=0.01 for T98G and p=0.00008 for U-87MG cell types, t-test) proliferation rate compared to cells generating dormant tumors (FIG. 4E). Invasion of T98G-F cells was 100-folds higher than that of T98G-D in fibrin 3D-bio-ink on day 14 (p=4×10⁻⁸, t-test), whereas invasion of U-87MG-F cells was 2-fold higher than U-87MG-D in fibrin 3D-bio-ink at day 14 (p=0.002, t-test) (FIG. 4F) in accordance to the in-vivo observations (29).

In addition, established and comprehensively characterized are pairs of dormant (Saos-2-D) and fast-growing (Saos-2-E) human osteosarcoma models in mice. Similarly to the GB dormancy models, Saos-2-E fast-growing cells generate tumors within a month following inoculation into SCID mice, while Saos-2-D dormant cells remain dormant for up to 7 months (40). However, when cultured in 2D, both dormant and fast-growing tumor-generating cells display similar growth rates. The growth rate of these additional cell types was evaluated in the fibrin 3D-bio-ink and found that their growth patterns resemble the in-vivo setting (p=0.0002, t-test).

These results suggest that the fibrin 3D-bio-ink could serve as a reliable ex-vivo model, preserving the in-vivo phenotype of a given tumor.

### EXAMPLE 5

### Therapeutic Effect of SELPi in Fibrin 3D-Bio-Ink Tumor Model

The differences in response to therapeutics in cells grown in 2D culture were evaluated in comparison to the fibrin 3D-bio-ink and in-vivo. For this purpose, the present inventors used a commercially available P-selectin inhibitor (SELPi), KF38789. SELPi did not affect the proliferation of PD-GB4 seeded in 2D cultures. However, a significant inhibition of the PD-GB4 cell proliferation (p=0.0001, t-test) were observed when cells were grown in fibrin 3D-bio-ink. The present inventors have recently observed a similar inhibitory effect in-vivo administering SELPi (16 mg/kg i.v. q.o.d.) to GB-bearing mice (36). It was speculated that these differences in response to treatment with SELPi correspond to the different expression levels of P-Selectin in 2D culture versus 3D. P-selectin is an adhesion molecule which was previously demonstrated to have a role in GB progression. It was previously found that elevated expression of P-Selectin is characteristic of GB 3D-spheroid models compared to 2D culture (28). Therefore, P-Selectin expression was evaluated in GB cells grown in the GB fibrin 3D-bio-ink. Flow cytometry analysis demonstrated high P-Selectin expression levels on PD-GB4 cells grown in fibrin 3D-bio-ink compared to 2D culture (FIG. 5A). Similar high expression of P-selectin in fibrin 3D-bio-ink compared to 2D culture was observed in additional four patient-derived GB cell types (not shown). Immunostaining for P-selectin showed positive expression in fibrin 3D-bio-ink composed of PD-GB4 cells in the presence or absence of hAstro and hMG (not shown) or in murine GB cells after 14 days in culture (not shown). SELPi did not affect two additional human GB cell types in 2D culture. Interestingly, significant inhibition of T98G cells (p=0.00003, t-test) and U-87MG (p=4×10⁻⁶, t-test) fast-growing cell types was obtained when cells were treated with SELPi in our fibrin 3D-bio-ink which correlates to the higher expression levels of P-selectin in 3D-bionk versus 2D culture (FIGS. 5B-C).

### EXAMPLE 6

### Higher Similarities in the Transcriptional Profile Between In-Vivo and 3D Compared to 2D Models

To further understand the changes attributed to the differences between 2D versus 3D cell cultures, the transcriptome of GB cells grown in 2D, in the 3D-bio-ink or isolated from GB tumors in mice was tested. RNA-seq of GB tumors grown in mice was performed and compared to penta-culture comprised of GL261 murine GB cells, primary astrocytes, microglia, pericytes, and endothelial cells, grown in 2D or in 3D-bio-ink (Methods). In order to specifically compare between tumor cells in all conditions, the present inventors focused on genes which have been previously shown to be intrinsically expressed in gliomas (41) (9511 genes out of 17810). The principal components analysis (PCA) (FIG. 6A) shows that the transcriptional signature of cells from the three conditions differ. PC1 which contains the most variance (56%) indicates that cells grown in fibrin 3D-bio-ink are more similar to the signature of GB tumor cells isolated from GB tumor bearing mice, as opposed to cells grown in 2D culture. To further investigate these differences, the Euclidian distance between the samples was calculated (FIG. 6B). This analysis, quantitively shows that the cells grown in the fibrin 3D-bio-ink are indeed more similar to the cells isolated from mice brain than the 2D culture samples (p=2.8×10⁷, FIG. 6C). Analysis of differentially expressed (DE) genes between the 3D and the 2D culture out of the 9511 glioma associated genes, identified 6936 differentially expressed genes. Gene set enrichment analysis (GSEA) shows several enriched pathways (according to their FDR q-value<0.05) including proliferation, cell-cell interaction, adhesion, inflammatory response, angiogenesis and several oncogenic markers. Among all enriched pathways, the present inventors focused on GB related pathways (angiogenesis hallmark, JAK-STAT signaling pathway, interferon gamma (IFN-γ) related response, VEGF pathway upregulation, EGFR pathway upregulation and TGF-β signaling pathway) and evaluated the upregulated genes in each examined group (FIG. 6D). The present inventors observed similar gene expression signature in cells grown in fibrin 3D-bio-ink as observed in cells isolated from intracranial GB tumors, suggesting that our system recapitulates various genetic programs that are also activated in-vivo. For example, RNA-seq analysis revealed activation of the JAK/STAT pathway suggesting that our system preserved GB-stroma interactions (42) better than the 2D model. The present inventors have also found enhance in the expression of the following genes both in fibrin 3D-bio-ink and iv-vivo: Jak3 and Socs7 which are known GB oncogenes (43), while Pik3cd was found to regulate GB migration (44) and Ifngr2 was found to be an immune modulator. Among the gene signature analysed, the present inventors found a significant upregulation in the gene expression of markers involved in macrophage recruitment (the chemoattractant CCL2 and ARG-1), angiogenesis (VEGFB and SPP1), matrix remodeling enzymes (MMP9) and genes related to endothelial cell junction molecules (PECAM1). Interestingly, it was found that RNA isolated from cells grown in our 3D-bio-ink expressed high levels of GB markers (GFAP, CHILI, NTN1) and of MPZL3, CD79b, PTPRN, RQS14, TIMP4 and NDRGI which were recently identified as potential poor-prognostic biomarkers of GB (45-47). Indeed, these genes were also found to be highly expressed in the RNA isolated from the in-vivo GB model.

### EXAMPLE 7

### Clinical Relevance

Temozolomide (TMZ), is an alkylating agent used as a standard of care chemotherapy for GB patients according to Stupp protocol (48). Therefore, the present inventors have evaluated the response of 3 patient-derived cells to TMZ when grown in 2D culture as well as in fibrin 3D-bio-ink (FIGS. 7A-C). The IC₅₀ values observed in 2D cultures were almost identical between the different patient-derived GB cells, however, each cell type exhibited different IC50 value when grown in the 3D model. This correlates with the fact that each patient responds differently to the same therapy and emphasizes the need for this personalized approach. Indeed, these patients showed a different response to TMZ and each of them survived for a different period of time.

As another evidence for the robustness and the broad possible uses of the system, the have established a metastatic breast cancer model (FIGS. 8A-B). The present inventors successfully created a vascularized 3D model of breast cancer brain metastasis, consisting of MDA-MB-231 human breast cancer cells, astrocytes, and microglia, as well as functional blood vessels prepared using human endothelial cells and pericytes. This is a proof of concept for the use of the 3D models for the study of brain metastasis in addition to GB primary models.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used. they should not be construed as necessarily limiting. In addition, any priority document(s) of this application is/are hereby incorporated herein by reference in its/their entirety.

### REFERENCES

### Other References are Cited Throughout the Application

ADDIN EN.REFLIST ADDIN EN.REFLIST 1. in www(dot)who(dot)int/news-room/fact-sheets/detail/cancer. (2018).
2. S. Jakka, M. Rossbach, An economic perspective on personalized medicine. The HUGO Journal 7, 1-6 (2013).
3. U. Ben-David et al., Genetic and transcriptional evolution alters cancer cell line drug response. Nature 560, 325-330 (2018).
4. J. A. DiMasi, H. G. Grabowski, R. W. Hansen, Innovation in the pharmaceutical industry: new estimates of R&D costs. Journal of health economics 47, 20-33 (2016).
5. Clinical Development Success Rates and Contributing Factors 2011-2020. BIO, QLS Advisors, Informa UK Ltd (2021).
6. S. N. Bhatia, D. E. Ingber, Microfluidic organs-on-chips. Nature biotechnology 32, 760-772 (2014).
7. G. J. Yoshida, Applications of patient-derived tumor xenograft models and tumor organoids. Journal of Hematology & Oncology 13, 4 (2020).
8. X. Dai, C. Ma, Q. Lan, T. Xu, 3D bioprinted glioma stem cells for brain tumor model and applications of drug susceptibility. Biofabrication 8, 045005 (2016).
9. Y. Zhao et al., Three-dimensional printing of Hela cells for cervical tumor model in vitro. Biofabrication 6, 035001 (2014).
10. P. DelNero, Y. H. Song, C. Fischbach, Microengineered tumor models: insights & opportunities from a physical sciences-oncology perspective. Biomedical microdevices 15, 583-593 (2013).
11. G. Trujillo-de Santiago et al., The Tumor-on-Chip: Recent Advances in the Development of Microfluidic Systems to Recapitulate the Physiology of Solid Tumors. Materials 12, 2945 (2019).
12. A. Sobrino et al., 3D microtumors in vitro supported by perfused vascular networks. Scientific reports 6, 31589 (2016).
13. S. Kim, H. Lee, M. Chung, N. L. Jeon, Engineering of functional, perfusable 3D microvascular networks on a chip. Lab on a Chip 13, 1489-1500 (2013).
14. T. Q. Huang, X. Qu, J. Liu, S. Chen, 3D printing of biomimetic microstructures for cancer cell migration. Biomedical microdevices 16, 127-132 (2014).
15. C. Box, S. J. Rogers, M. Mendiola, S. A. Eccles, in Seminars in cancer biology. (Elsevier, 2010), vol. 20, pp. 128-138.
16. Y. S. Zhang et al., Bioprinting the cancer microenvironment. ACS biomaterials science & engineering 2, 1710-1721 (2016).
17. M. A. Heinrich et al., 3D-bioprinted mini-brain: a glioblastoma model to study cellular interactions and therapeutics. Advanced materials 31, 1806590 (2019).
18. H. Lodish et al., Molecular cell biology. (Macmillan, 2008).
19. M. A. Skylar-Scott et al., Biomanufacturing of organ-specific tissues with high cellular density and embedded vascular channels. Science advances 5, eaaw2459 (2019).
20. L. Ma et al., Towards personalized medicine with a three-dimensional micro-scale perfusion-based two-chamber tissue model system. Biomaterials 33, 4353-4361 (2012).
21. F. Meng et al., 3D bioprinted in vitro metastatic models via reconstruction of tumor microenvironments. Advanced Materials 31, 1806899 (2019).
22. M. S. Ozturk et al., High-resolution tomographic analysis of in vitro 3D glioblastoma tumor model under long-term drug treatment. Science Advances 6, eaay7513 (2020).
23. N. El Demerdash, J. Kedda, N. Ram, H. Brem, B. Tyler, Novel therapeutics for brain tumors: current practice and future prospects. Expert Opinion on Drug Delivery 17, 9-21 (2020).
24. E. Yeini et al., Targeting Glioblastoma: Advances in Drug Delivery and Novel Therapeutic Approaches. Advanced Therapeutics, 2000124.
25. E. Segal et al., Targeting angiogenesis-dependent calcified neoplasms using combined polymer therapeutics. PLOS One 4, e5233 (2009).
26. O. Yom-Tov, L. Neufeld, D. Seliktar, H. Bianco-Peled, A novel design of injectable porous hydrogels with in situ pore formation. Acta biomaterialia 10, 4236-4246 (2014).
27. L. Neufeld, H. Bianco-Peled, Designing a biocompatible hydrogel for the delivery of mesalamine. International journal of pharmaceutics 491, 170-179 (2015).
28. S. Ferber et al., Co-targeting the tumor endothelium and P-selectin-expressing glioblastoma cells leads to a remarkable therapeutic outcome. Elife 6, (2017).
29. R. Satchi-Fainaro et al., Prospective identification of glioblastoma cells generating dormant tumors. PLOS One 7, e44395 (2012).
30. N. Bray, H. Pimentel, P. Melsted, L. Pachter. (ISSN).
31. B. Li, C. N. Dewey, RSEM: accurate transcript quantification from RNA-Seq data with or without a reference genome. BMC bioinformatics 12, 323 (2011).
32. S. Anders, W. Huber, Differential expression analysis for sequence count data. Nature Precedings, 1-1 (2010).
33. P. A. Netti, D. A. Berk, M. A. Swartz, A. J. Grodzinsky, R. K. Jain, Role of extracellular matrix assembly in interstitial transport in solid tumors. Cancer research 60, 2497-2503 (2000).
34. N. Rahimi, D. G. Molin, T. J. Cleij, M. A. van Zandvoort, M. J. Post, Electrosensitive polyacrylic acid/fibrin hydrogel facilitates cell seeding and alignment. Biomacromolecules 13, 1448-1457 (2012).
35. C. S. von Bartheld, J. Bahney, S. Herculano-Houzel, The search for true numbers of neurons and glial cells in the human brain: A review of 150 years of cell counting. Journal of Comparative Neurology 524, 3865-3895 (2016).
36. E. Yeini, P-Selectin axis plays a key role in microglia immunophenotype and glioblastoma progression. (2020).
37. D. Matias et al., Microglia/astrocytes-glioblastoma crosstalk: crucial molecular mechanisms and microenvironmental factors. Frontiers in cellular neuroscience 12, 235 (2018).
38. G. Tiram et al., Reverting the molecular fingerprint of tumor dormancy as a therapeutic strategy for glioblastoma. The FASEB Journal 32, 5835-5850 (2018).
39. N. Almog et al., Transcriptional switch of dormant tumors to fast-growing angiogenic phenotype. Cancer research 69, 836-844 (2009).
40. G. Tiram et al., Identification of dormancy-associated microRNAs for the design of osteosarcoma-targeted dendritic polyglycerol nanopolyplexes. ACS nano 10, 2028-2045 (2016).
41. Q. Wang et al., Tumor evolution of glioma-intrinsic gene expression subtypes associates with immunological changes in the microenvironment. Cancer cell 32, 42-56. e46 (2017).
42. D. H. Heiland et al., Tumor-associated reactive astrocytes aid the evolution of immunosuppressive environment in glioblastoma. Nature communications 10, 1-12 (2019).
43. S. Nguyen, Graduate Studies, (2013).
44. Z. Azam et al., Comprehensive RNAseq analysis reveals PIK3CD promotes glioblastoma tumorigenesis by mediating PI3K-Akt signaling pathway. Glioma 3, 135 (2020).
45. M. Tang et al., Rapid 3D Bioprinting of Glioblastoma Model Mimicking Native Biophysical Heterogeneity. Small, 2006050 (2021).
46. M. Tang et al., Three-dimensional bioprinted glioblastoma microenvironments model cellular dependencies and immune interactions. Cell Research, (2020).
47. W. Yin et al., Expression profile analysis identifies a novel five-gene signature to improve prognosis prediction of glioblastoma. Frontiers in genetics 10, 419 (2019).
48. R. Stupp et al., Radiotherapy plus concomitant and adjuvant temozolomide for glioblastoma. New England Journal of Medicine 352, 987-996 (2005).
49. C. Unger et al., Modeling human carcinomas: physiologically relevant 3D models to improve anti-cancer drug development. Advanced drug delivery reviews 79, 50-67 (2014).
50. D. T. Butcher, T. Alliston, V. M. Weaver, A tense situation: forcing tumour progression. Nature Reviews Cancer 9, 108-122 (2009).
51. M. J. Paszek et al., Tensional homeostasis and the malignant phenotype. Cancer cell 8, 241-254 (2005).
52. S. Krishnamoorthy, B. Noorani, C. Xu, Effects of Encapsulated Cells on the Physical-Mechanical Properties and Microstructure of Gelatin Methacrylate Hydrogels. International Journal of Molecular Sciences 20, 5061 (2019).
53. K. Chen et al., An All-in-One Tannic Acid-Containing Hydrogel Adhesive with High Toughness, Notch Insensitivity, Self-Healability, Tailorable Topography, and Strong, Instant, and On-Demand Underwater Adhesion. ACS Applied Materials & Interfaces, (2021).
54. S. Naghich, M. Sarker, N. Sharma, Z. Barhoumi, X. Chen, Printability of 3D Printed Hydrogel Scaffolds: Influence of Hydrogel Composition and Printing Parameters. Applied Sciences 10, 292 (2020).
55. C. Schultz, N. Lemke, S. Ge, W. A. Golembieski, S. A. Rempel, Secreted protein acidic and rich in cysteine promotes glioma invasion and delays tumor growth in vivo. Cancer research 62, 6270-6277 (2002).
56. C. Wang, X. Tong, F. Yang, Bioengineered 3D brain tumor model to elucidate the effects of matrix stiffness on glioblastoma cell behavior using PEG-based hydrogels. Molecular pharmaceutics 11, 2115-2125 (2014).
57. H. Schwartz et al., Incipient melanoma brain metastases instigate astrogliosis and neuroinflammation. Cancer research 76, 4359-4371 (2016).
58. D. B. Kolesky, K. A. Homan, M. A. Skylar-Scott, J. A. Lewis, Three-dimensional bioprinting of thick vascularized tissues. Proc Natl Acad Sci U S A 113, 3179-3184 (2016).
59. H .- G. Yi et al., A bioprinted human-glioblastoma-on-a-chip for the identification of patient-specific responses to chemoradiotherapy. Nat. Biomed. Eng 1, (2019).
60. A. Eldar-Boock et al., Integrin-assisted drug delivery of nano-scaled polymer therapeutics bearing paclitaxel. Biomaterials 32, 3862-3874 (2011).
61. M. De Palma, D. Biziato, T. V. Petrova, Microenvironmental regulation of tumour angiogenesis. Nature Reviews Cancer 17, 457 (2017).
62. A. Yoshino et al., Therapeutic implications of interferon regulatory factor (IRF)-1 and IRF-2 in diffusely infiltrating astrocytomas (DIA): response to interferon (IFN)-β in glioblastoma cells and prognostic value for DIA. Journal of neuro-oncology 74, 249-260 (2005).
63. J. Liang, Y. Piao, V. Henry, N. Tiao, J. F. de Groot, Interferon-regulatory factor-1 (IRF1) regulates bevacizumab induced autophagy. Oncotarget 6, 31479 (2015).
64. I. Manini et al., Semaphorin-7A on Exosomes: A Promigratory Signal in the Glioma Microenvironment. Cancers 11, 758 (2019).
65. C. Angelucci, G. Lama, G. Sica, Multifaceted Functional Role of Semaphorins in Glioblastoma. International journal of molecular sciences 20, 2144 (2019).
66. Y .- S. Liu et al., MiR-181b modulates EGFR-dependent VCAM-1 expression and monocyte adhesion in glioblastoma. Oncogene 36, 5006-5022 (2017).
67. C. D. Schmid et al., GPR182 is a novel marker for sinusoidal endothelial differentiation with distinct GPCR signaling activity in vitro. Biochemical and biophysical research communications 497, 32-38 (2018).
68. V. Ramachandran et al., Adrenomedullin is expressed in pancreatic cancer and stimulates cell proliferation and invasion in an autocrine manner via the adrenomedullin receptor, ADMR. Cancer research 67, 2666-2675 (2007).
69. H. Doron, T. Pukrop, N. Erez, A Blazing Landscape: Neuroinflammation Shapes Brain Metastasis.Cancer Res 79, 423-436 (2019).
70. F. Sivandzade, L. Cucullo, In-vitro blood-brain barrier modeling: A review of modern and fast-advancing technologies. Journal of Cerebral Blood Flow & Metabolism 38, 1667-1681 (2018).

What is claimed is:

1. A three dimensional (3D) model of a glioblastoma (GB) tumor or a brain metastasis comprising a hardened first synthetic material and a plurality of cell types of said tumor or said brain metastasis and microenvironment thereof, said plurality of cell types comprising endothelial cells, pericytes, astrocytes, microglia cells and GB tumor cells or metastatic cells.

2. The 3D model of claim 1, wherein a first mixture of said plurality of cell types form said tumor and a microenvironment thereof, and a second mixture of said plurality of cell types form a vasculature which allows perfusion, said first and second mixtures being different from one another.

3. The 3D model of claim 1, wherein said endothelial cells and pericytes form a vasculature which allows perfusion and optionally wherein a ratio of said endothelial cells and said pericytes in said vasculature ranges from 2:1 to 10:1.

4. The 3D model of claim 1 characterized by at least one of:
(i) porosity of 1-40 μm;
(ii) cell viability for at least 4 weeks under physiological conditions;
(iii) stiffness of 5-25 kPa Young's Modulus;
(iv) swelling equilibrium of 5-25%;
(v) perfusability for at least 1 day; and/or (vi) activation of said microglia and/or astrocytes.

5. The 3D model of claim 2, wherein said vasculature is configured to provide perfusion at a range of shear stress of 0.01-100 dyn/cm2 or 5-1000 μl/min, for a vessel of 1 mm in diameter of said vasculature and/or wherein said vasculature is configured to provide perfusion at shear stress of 25 μl/min for a vessel 1 mm in diameter of said vasculature and/or wherein said vasculature is for perfusing peripheral blood cells (PBMCs) and/or a drug.

6. The 3D model of claim 1, wherein said tumor cells are non-immortalized patient derived or wherein said tumor cells are of a GB cell line.

7. The 3D model of claim 1, wherein a density of said tumor cells in the model is between is between 0.1×10⁵-1×10⁸ cells/ml;
wherein a ratio of said tumor cells to said astrocytes and microglia cells is in the range of 20:1 to 1:10;
wherein a ratio of said tumor cells to said astrocytes and microglia cells is in the range of 1:10 to 10:1; and/or wherein a ratio of said tumor cells to said astrocytes and microglia cells is in the range of 1:1 to 5:1.

8. The 3D model of claim 1, being embedded in an extracellular matrix.

9. The 3D model of claim 1, wherein said first synthetic material comprises a hardened form of a curable material, or wherein said first hardened synthetic material comprises a polymeric material, or wherein said first hardened synthetic material comprises fibrin and an anionic polymer cross-linked to one another.

10. The 3D model of claim 1, wherein said first synthetic material comprises fibrin and an anionic polymer cross-linked to one another, optionally wherein said fibrin is formed upon enzymatically-catalyzed polymerization of fibrinogen, optionally wherein said fibrin and said anionic polymer are cross-linked to one another upon an enzymatic reaction.

11. The 3D model of claim 10, wherein said anionic polymer comprises gelatin and/or wherein an amount of said anionic polymer, an amount of said fibrin and a degree of said cross-linking are selected so as to provide a pre-determined mechanical and/or physical property of said GB tumor model (said property being selected, for example, in accordance with a respective property of a patient's derived glioblastoma tumor).

12. The 3D model of claim 10, wherein said first synthetic material is formed from a curable formulation that comprises said anionic polymer and fibrinogen optionally wherein said curable formulation further comprises at least one enzyme for promoting said cross-linking and/or for generating said fibrin, optionally wherein a concentration of said anionic polymer (e.g., gelatin) in said curable formulation ranges from 1 to 20, or from 3 to 18, or from 3 to 10, or from 1 to 10, or from 4 to 10, or is 6, % by weight of the total weight of the curable formulation, optionally wherein a concentration of said fibrinogen in said curable formulation ranges from 0.1 to 10, or from 0.1 to 5, or from 0.5 to 5, or from 1 to 5, or from 0.1 to 3, or from 0.5 to 2, or from 0.5 to 1.5, or is 1, % by weight of the total weight of the curable formulation.

13. The 3D model of claim 12, wherein said enzyme is selected from thrombin, transglutaminase and a mixture thereof, optionally wherein a concentration of said thrombin, if present, in said curable formulation, ranges from 0.1 to 5, or from 0.1 to 4, or from 0.1 to 3, or from 0.1 to 2, or from 0.1 to 1, or is 0.5, U/ml or wherein a concentration of said transglutaminase, if present, in said curable formulation, ranges from 0.1 to 50, 0.1 to 10, or from 1 to 10, or from 1 to 5, or is 3, % by volume of the total volume of the formulation.

14. The 3D model of claim 13, wherein said first synthetic material comprises fibrin, gelatin, thrombin and transglutaminase (TG).

15. The 3D model of claim 2, wherein said vasculature comprises perfused immune cells or a perfused drug.

16. A curable formulation, comprising fibrinogen, gelatin, thrombin and transglutaminase.

17. A method of producing the 3D model of claim 1 comprising:
(a) dispensing at least one layer of astrocytes, microglia cells and GB tumor cells or metastatic cells comprised in a first synthetic material;
(b) forming a vascularization pattern on said at least one layer using a second synthetic material which is liquefiable to allow formation of perfusable tubular vessels comprising lumen;
wherein step (a) is performed prior to and following step (b);
(c) dispensing said second synthetic material to form said lumen;
(d) applying endothelial cells and pericytes into said lumen to form blood vessels.

18. A system comprising the perfused 3D model of claim 1 and a container in fluid communication with the 3D model of claim 1.

19. A method of screening for an anti-cancer treatment regimen suitable for a patient suffering from glioblastoma, the method comprising:
subjecting a 3D model of a tumor according to claim 1 to said anti-cancer treatment regimen; and
determining a presence of an anti-cancer effect of said anti-cancer treatment regimen at a personalized manner.

20. Use of the 3D model of claim 2 in drug screening, drug development or target discovery.
